(19) United States
(12) Patent Application Publication
(10) Pub. No.: US 2021/0254097 A1
(43) Pub. Date: Aug. 19, 2021

(54) ENGINEERING OF IMMUNE CELLS FOR EX VIVO CELL THERAPY APPLICATIONS

(71) Applicant: Avectas Limited, Co. Kildare (IE)

(72) Inventors: Shirley O'Dea, Maynooth (IE); Michael Maguire, Dublin (IE)

(21) Appl. No.: 17/012,748

(22) Filed: Sep. 4, 2020

Related U.S. Application Data
(60) Provisional application No. 63/047,054, filed on Jul. 1, 2020.
(60) Provisional application No. 63/022,944, filed on May 11, 2020.
(60) Provisional application No. 62/897,250, filed on Sep. 6, 2019.

(51) Int. Cl. C12N 15/85; C07K 14/705; C12N 5/078; A61K 35/17

(52) U.S. Cl. CPC C12N 15/85; A61K 35/17; C12N 5/0634; C07K 14/70596

(57) ABSTRACT

The invention provides a solution to the problem of transfecting non-adherent cells. Methods and compositions containing ethanol and an isotonic salt solution are used for delivery of compounds and compositions to non-adherent cells, e.g. T cells.

### RELATED APPLICATIONS

This application claims the benefit of priority under 35 U.S.C. § 119(e) to U.S. Provisional Application No. 62/897,250 filed Sep. 6, 2019, U.S. Provisional Application No. 63/022,944 filed May 11, 2020, and U.S. Provisional Application No. 63/047,054 filed Jul. 1, 2020, the entire contents of each of which is incorporated herein by reference in their entireties.

### INCORPORATION-BY-REFERENCE OF SEQUENCE LISTING

The contents of the sequence listing text file named “48831-521001US_Sequence_Listing_ST25.txt”, which was created on Dec. 2, 2020 and is 122,880 bytes in size, is hereby incorporated by reference in its entirety.

### FIELD OF THE INVENTION

The invention relates to the delivery of agents into mammalian cells for immunotherapy.

### BACKGROUND

Variability in cell transfection efficiency exists among different cell types. Transfection of suspension cells, e.g., non-adherent cells, has proven to be difficult using conventional methods. Thus, a need exists for compositions and methods to facilitate transfection of such cells. Moreover, there is a need for improved manufacturing strategies aimed at ensuring immune cell potency and cellular therapy.

### SUMMARY OF THE INVENTION

The invention provides a solution to engineering immune cells for ex vivo cell therapy applications. The compositions and methods described herein facilitate cell engineering technologies that enable next generation cell therapy products which require complex modifications and high levels of cell functionality. As described herein, the SOLUPORE™ delivery method is a non-viral means of simply, rapidly and efficiently delivering cargos to primary immune cells, while retaining cell viability and functionality. Moreover, these engineered immune cells, e.g., T-cells, reduce likelihood of T cell exhaustion, thus enabling the their use for complex therapeutic needs.

Accordingly, provided herein is an immune cell (or population of immune cells), e.g., a T-cell, natural killer (NK cell), B-cell, macrophage, or other immune cell, comprising an exogenous cargo, wherein the immune cell with the exogenous cargo has a molecular profile that has an expression level of a gene or protein within a log 2 fold change of 3 at 24 hours post cargo delivery compared to the level of the gene or protein in a control immune cell at 24 hours post cargo delivery. The gene or protein is a member of the Activator Protein 1 (AP-1) signal transduction pathway, and the molecular profile is independent of the type of cargo delivered. A control immune cell is an immune cell that has not experienced a cell engineering process or cell activation step. For example, the control immune cell has not been manipulated using electroporation methods, viral transduction methods, or other methods (including SOLUPORE™ methods) to deliver cargo into the cell.

For example, the molecular profile of the immune cell which comprises the exogenous cargo has an expression of a gene or protein within a log₂ fold change of 3, or within a log₂ fold change of 2, or within a log₂ fold change of 1 compared to the level of the gene or protein in a control immune cell. For example, the molecular profile (gene expression profile) is assessed at or about 6 hours, 12 hours, 24 hours, 36 hours, 48 hours, 72 hours, or 96 hours, post cargo delivery. Alternatively, the molecular profile of the immune cell comprising the exogenous cargo has an expression of a gene or protein within a log₂ fold change of 3, or within a log₂ fold change of 2, or within a log₂ fold change of 1 of the level of the gene or protein in a control immune cell at 1 day, 2 days, 3 days, 4 days, 5 days, 6 days, 7 days, 8 days, 9 days, 10 days, 11 days, 12 days, 13 days, 14 days 1 week, 2 weeks, 4 weeks, 1 month, 2 months, 3 months, or 4 months post cargo delivery.

A cell engineering process may include electroporation, a process in which an electrical field is applied to cells to increase the cell membrane permeability (also called electrotransfer). Moreover, the cell engineering process may refer to any known transfection method for intracellular delivery, including the SOLUPORE™ delivery method, membrane-disrupting methods (electroporation, sonoporation, magnetotection, optoperation), or carrier-based methods (e.g., lipid nanoparticles).

The molecular profile refers to gene expression, the genomic profile, protein expression, protein activity, or the proteomic profile of the immune cell. In other examples, the immune cell with the exogenous cargo has a molecular profile that has an expression level of a gene or protein within log₂ fold change of 2 of the level of the gene or protein in the control immune cell. In further examples, the immune cell with the exogenous cargo has a molecular profile that has an expression level of a gene or protein within log₂ fold change of 1 of the level of the gene or protein in the control immune cell.

In examples, the exogenous cargo of the immune cell comprises a nucleic acid, a small molecule, a protein, a polypeptide, or a combination thereof. For example, the nucleic acid comprises messenger ribonucleic acid (mRNA), small interfering RNA (siRNA), short hairpin RNA (shRNA), deoxyribonucleic acid (DNA), or any combination thereof.

The exogenous cargo, or “payload” are terms used to describe a compound, e.g., a nucleic acid comprising mRNA, or composition that is delivered via an aqueous solution across a cell plasma membrane and into the interior of a cell.

An immune cell of the invention, e.g., the immune cell having the exogenous cargo, has a molecular profile where Fos (v-fos FBJ murine osteosarcoma viral oncogene homolog, FBJ murine osteosarcoma viral oncogene homolog), Jun v-jun avian sarcoma virus 17 oncogene homolog) or combinations thereof are expressed at a level within a log₂ fold change of 3 of the level expressed in the control immune cell. For example, the immune cell of the invention, e.g., the immune cell having the exogenous cargo, has a molecular profile where Fos, Jun or combinations thereof are expressed at a level about a log₂ fold change of −3 compared to the level expressed in control immune cell.

In other examples, the immune cell of the invention, e.g., the immune cell having the exogenous cargo, has a molecular profile where Fos, Jun or combinations thereof are expressed at a level within a log₂ fold change of 2 of the level expressed in the control immune cell. In further examples, the immune cell of the invention, e.g., the immune cell having the exogenous cargo, has a molecular profile where Fos, Jun or combinations thereof are expressed at a level within a log₂ fold change of 1 compared to the level expressed in the control immune cell.

In some embodiments, Fos comprises human Fos comprising the exemplary nucleic acid sequence of SEQ ID NO: 1. In some embodiments, Jun comprises human Jun comprising the exemplary nucleic acid sequence of SEQ ID NO: 2.

The immune cell of the invention, e.g., the immune cell having the exogenous cargo, has a molecular profile where Fos, Jun, FosB (FBJ murine osteosarcoma viral oncogene homolog B; SEQ ID NO: 3), BATF (Basic leucine zipper transcription factor ATF-like), BATF (Basic leucine zipper transcription factor ATF-like; SEQ ID NO: 4), BATF3 (Basic leucine zipper transcriptional factor ATF-like 3; SEQ ID NO: 5), or combinations thereof are expressed at a level within a log₂ fold change of 3, a log₂ fold change of 2, or a log 2 fold change of 1 compared to the level expressed in a control immune cell (an immune cell not having the exogenous cargo). In embodiments, the immune cell of the invention, e.g., the immune cell having the exogenous cargo, has a molecular profile where Fos, Jun, FosB, BATF, or BATF3 are expressed at a level within about log₂ fold change of −3, log₂ fold change of −2 or log₂ fold change of −1 compared to the level expressed in a control immune cell (an immune cell that has not experienced a cell engineering process or cell activation step). For example (a negative number), the gene is expressed at a level less than that of the control immune cell.

In examples, the immune cell of the invention, e.g., the immune cell having the exogenous cargo, has a molecular profile where Fos, Jun, FosB, BATF, or BATF3 are expressed at a level within a log 2 fold change of 1 compared the level expressed in the control immune cell. In examples, the immune cell of the invention, e.g., the immune cell having the exogenous cargo, has a molecular profile where Fos, Jun, FosB, BATF, or BATF3 are expressed at a level within a log₂ fold change of 2 compared to the level expressed in a control immune cell. In examples, the immune cell of the invention, e.g., the immune cell having the exogenous cargo, has a molecular profile where Fos, Jun, FosB, BATF, or BATF3 are expressed at a level within a log₂ fold change of 3 compared to the level expressed in a control immune cell.

In embodiments, the exogenous cargo includes nucleic acid. For example, the cargo includes messenger ribonucleic acid (mRNA). For example, the mRNA encodes a chimeric antigen receptor (CAR). For example, the CAR targets CD19 (cluster of differentiation 19). An exemplary mRNA encoding CD19 CAR comprises the amino acid sequence of SEQ ID NO: 6 or SEQ ID NO: 8.

In other embodiments, the mRNA encodes TRAIL-DR5 (TNF-related apoptosis-inducing ligand (TRAIL) Death Receptor 5) variant mRNA (SEQ ID NO: 10), TRAIL DNA (SEQ ID NO: 11), see for example, U.S. Pat. No. 7,994,281, incorporated herein by reference in its entirety. In other embodiments, the mRNA encodes IL-15 (interleukin 15) mRNA or TCR (T cell receptor) mRNA.

In other examples, the exogenous cargo comprises Cas9 (CRISPR associated protein 9) protein, for example with guide RNAs including TRAC (T cell receptor alpha constant) or PD-1 (programmed death ligand 1). For example, the Cas9 protein sequences comprise SEQ ID NO: 19, SEQ ID NO: 20 or SEQ ID NO: 21. The sequence for human TRAC targeting gRNA comprises SEQ ID NO: 25. The sequence for human PDCD1 targeting gRNA comprises SEQ ID NO: 26.

In other examples, the exogenous cargo comprises Cas12a protein (CRISPR associated protein 12a) including guide RNAs including TRAC and PD-1. The Cas12a protein sequences comprise SEQ ID NO: 22, SEQ ID NO: 23 or SEQ ID NO: 24. In examples, the exogenous cargo comprises MAD7 protein, with guide RNAs including TRAC or PD-1. In examples, the exogenous cargo comprises SgCas, with guide RNAs including TRAC or PD-1. In examples, the exogenous cargo comprises Cas13, with guide RNAs including TRAC or PD-1. Alternatively, the exogenous cargo comprises base editors such as Cas9n, or zinc finger nucleases, or MegaTALs.

In examples, the exogenous cargo comprises the Sleeping Beauty 100 transposon/transposase system, or the Sleeping Beauty 1000 transposon/transposase system, or the Piggy Bac transposon/transposase system, or the TcBuster transposon/transposase system.

In other examples, the exogenous cargo comprises DNA, for example, CD19 CAR DNA, TRAIL DNA, or IL-15 DNA.

In examples, the exogenous cargo comprises the Yamanaka factors used for generation of stable induced pluripotent stem cells from adult human cells. For example, the Yamanaka factors comprise c-Myc (MYC proto-oncogene, bHLH transcription factor, SEQ ID NO: 13), Klf4 (Kruppel Like Factor 4, SEQ ID NO: 14), Oct4 (octamer-binding transcription factor 4, SEQ ID NO: 15), or Sox2 (SRY (sex determining region Y)-box 2, SEQ ID NO: 16).

In further examples, the exogenous cargo comprises siRNA (small interfering RNA), for example against PD-1. In further examples, the exogenous cargo comprises shRNA (short hairpin RNA), for example against PD-1.

The term, “exogenous” refers to cargo (or payload) coming from or deriving from outside the cell, e.g., an immune cell, as opposed to an endogenous agent that originated within the immune cell.

Various methods may be utilized to characterize the molecular profile of the immune cells. For example, the molecular profile may be done using DNA analysis, RNA analysis, protein analysis, cytokine analysis, or combinations thereof. For example, the molecular profile occurs by RNA analysis. In some embodiments, the RNA analysis includes RNA quantification. In some embodiments, the RNA quantification occurs by reverse transcription quantitative PCR (RT-qPCR), multiplexed qRT-PCR, fluorescence in situ hybridization (FISH), and combinations thereof. In some embodiments, the molecular profile analysis occurs by DNA analysis. In some embodiments, the DNA analysis includes amplification of DNA sequences from one or more identified cells. In some embodiments, the amplification occurs by the polymerase chain reaction (PCR). In some embodiments, the molecular profile occurs by RNA or DNA sequencing. In some embodiments, the RNA or DNA sequencing occurs by methods that include, without limitation, whole transcriptome analysis, whole genome analysis, barcoded sequencing of whole or targeted regions of the genome, and combinations thereof. In other examples, the molecular profile occurs by protein analysis, including for example an at the proteomic level.

In embodiments, the immune cell having the exogenous cargo has a molecular profile that has an expression level of a gene or protein (e.g., in the AP—signaling pathway) about a log₂ fold change of −3, a log₂ fold change of −2, or a log₂ fold change of −1 compared to the level of the gene or protein in a control immune cell. For example, the immune cell having the exogenous cargo has a molecular profile that has an expression level of a gene or protein in the AP-1 signaling pathway about a log₂ fold change of −1 compared to the level of the gene or protein in the control immune cell. For example, the immune cell having the exogenous cargo has a molecular profile that has an expression of a gene or protein in the AP-1 signaling pathway about a log₂ fold change of −2 compared to the level of the gene or protein in the control immune cell. In other examples, the immune cell having the exogenous cargo has a molecular profile that has an expression of a gene or protein about a log₂ fold change of −1 compared to the level of the gene or protein in the control immune cell.

In embodiments, the immune cell having the exogenous cargo has a molecular profile with an expression level of a gene or protein that is within a log₂ fold change of 3, a log₂ fold change of 2, or a log 2 fold change of 1 compared to the level of the gene or protein in a control immune cell, and the gene or protein in the AP-1 (activator protein 1) signaling pathway. AP-1 is a transcription factor that regulates gene expression in response to a variety of stimuli, including cytokines, growth factors, stress, and bacterial and viral infections. AP-1 controls a number of cellular processes including differentiation, proliferation, and apoptosis. For example, exhausted T cells exhibit low expression of AP-1 factors, including Fos, Jun, and/or Fosb (FBJ murine osteosarcoma viral oncogene homolog B). For example, Fos has human nucleic acid sequence of SEQ ID NO: 1. In other examples, Jun comprises the nucleic acid sequence of SEQ ID NO: 2.

In some embodiments, the immune cell of the invention comprises at least two or more exogenous cargos (e.g., 3, 4, 5, 6 7, 8, 9, or 10 exogenous cargos). The exogenous cargo comprises a nucleic acid (for example, RNA (ribonucleic acid), mRNA (messenger RNA), or DNA (deoxyribonucleic acid)), a protein or peptide, a small chemical molecule, or any combination thereof.

The immune cell of the invention (e.g., the immune cell having the exogenous cargo) is associated with numerous advantages, e.g., the immune cells processed using the SOLUPORE™ method exhibit few or no phenotypic characteristics of T cell exhaustion or T cell anergy. T cell anergy is a dysfunctional state of T cells stimulated in the absence of co-stimulatory signals. T cell exhaustion refers to a progressive loss of T cell effector function due to prolonged antigen stimulation. Furthermore, T cell stimulation refers to the engagement of T-cell receptor (TCR)/CD3 (cluster of differentiation 3) complexes and costimulatory receptors such as CD28 (cluster of differentiation 28), which leads to activation of the cell. Since cells processed by the SOLUPORE™ method show few or no phenotypic characteristics of T cell exhaustion or anergy, they are better suited for clinical use, i.e. their immune function is preserved and therefore confer a superior clinical benefit compared electroporated cells.

T-cell “exhaustion” is describes the state of T cells that respond poorly because of prolonged antigen exposure during chronic viral infections or cancer or other manipulations such as prolonged engagement of cell surface receptors, e.g CD3 or CD28 with a ligand such an anti-CD3 antibody or anti-CD28 antibody. “T cell exhaustion” is characterized by loss of T cell function. Exhausted T cells display a transcriptional profile distinct from that of functional effector or memory T cells, characterized by the expression of inhibitory cell surface receptors including PD-1, LAG-3 (Lymphocyte-activation gene 3), TIM-3 (T-cell immunoglobulin mucin-3), TIGIT (T cell immunoreceptor with Ig and ITIM domains), and CTLA-4 (cytotoxic T-lymphocyte-associated protein 4), IL-2 (interleukin 2), TNF (tumor necrosis factor), and IFN-γ (interferon gamma) cytokine production. NFAT (Nuclear factor of activated T-cells) and AP-1 transcription factors synergistically play a central role in inducing hyporesponsive states, such as anergy and exhaustion. Exhausted cells exhibit low expression of AP-1 factors (FOS, FOSB, and Jun). Additionally, T cell anergy may refer to a tolerance mechanism in which the lymphocyte is intrinsically functionally inactivated following an antigen encounter, but remains alive for an extended period of time in a hyporesponsive state.

In embodiments, the immune cell of the invention (e.g., the immune cell having the exogenous cargo) comprises an unstimulated immune cell. For example, the immune cell is not stimulated with a ligand of CD3, CD28, or a combination thereof. Put another way, the immune cell is not contacted with a CD3 or CD28 ligand, for example, an antibody or antibody fragment that binds to CD3, CD28, or both.

In aspects, the immune cell of the invention (e.g., the immune cell having the exogenous cargo) secretes at least one cytokine at a level within a log₂ fold change of 3 compared to the level of that an immune cell that has not experienced a cell engineering process. In other embodiments, the immune cell of the invention (e.g., the immune cell having the exogenous cargo) secretes at least one cytokine within a log₂ fold change of 2 compared to the level of an immune cell that has not experienced a cell engineering process. In other embodiments, the immune cell of the invention (e.g., the immune cell having the exogenous cargo) secretes at least one cytokine at a level within a log₂ fold change of 1 compared to the level of the an immune cell that has not experienced a cell engineering process. For example, the immune cell of the invention does not cause non-specific secretion (also referred to as “release” and refers to cytokine release from a cell, e.g., an immune cell) of cytokines, as compared to a control immune cell.

In embodiments, the immune cell of the invention (e.g., the immune cell having the exogenous cargo) secretes the cytokine IL-2 (interleukin 2) and/or or IL-8 (interleukin 8) at a level within a log₂ fold change of 3 compared an immune cell that has not experienced a cell engineering process. In embodiments, the immune cell of the invention (e.g., the immune cell having the exogenous cargo) secretes the cytokine IL-2 or IL-8 at a level within a log₂ fold change of 2 compared an immune cell that has not experienced a cell engineering process. In embodiments, the immune cell of the invention (e.g., the immune cell having the exogenous cargo) secretes the cytokine IL-2 or IL-8 at a level within a log₂ fold change of 1 compared an immune cell that has not experienced a cell engineering process.

In embodiments, the immune cell of the invention (e.g., the immune cell having the exogenous cargo) secretes the cytokine IL-2 or IL-8 at a level about a log₂ fold change of −3 compared to an immune cell that has not experienced a cell engineering process.

In embodiments, the immune cell of the invention (e.g., the immune cell having the exogenous cargo) secretes the cytokine IL-2 or IL-8 at a level about a log₂ fold change of −2 compared to an immune cell that has not experienced a cell engineering process. In embodiments, the immune cell of the invention (e.g., the immune cell having the exogenous cargo) secretes the cytokine IL-2 or IL-8 at a level about a log₂ fold change of −1 compared to an immune cell that has not experienced a cell engineering process.

In embodiments, the IL-2 (e.g., human IL-2) comprises the nucleic acid sequence of SEQ ID NO: 17. In other embodiments, the IL-8 (e.g., human IL-8) comprises the nucleic acid sequence of SEQ ID NO: 18.

In embodiments, the immune cell of the invention (e.g., the immune cell having the exogenous cargo) secretes the cytokine IFN-γ (interferon gamma), IL-2, TNFα (tumor necrosis factor alpha), IL-8, GM-CSF (Granulocyte-macrophage colony-stimulating factor), IL-10 (interleukin 10), MIP-1α (macrophage inflammatory protein 1 alpha), MIP-1β (macrophage inflammatory protein 1 beta), IL-17A (interleukin 17A), Fractalkine, or ITAC (Interferon—inducible T Cell Alpha Chemoattractant) at a level at a level within a log₂ fold change of 3 compared to an immune cell that has not experienced a cell engineering process. In other examples, the immune cell of the invention (e.g., the immune cell having the exogenous cargo) secretes the cytokine IFN-γ, IL-2, TNFα, IL-8, GM-CSF, IL-10, MIP-1α, MIP-1β, IL-17A, Fractalkine, ITAC, or combinations thereof at a level at a level within a log₂ fold change of 2 compared to an immune cell that has not experienced a cell engineering process. In other examples, the immune cell of the invention (e.g., the immune cell having the exogenous cargo) secretes the cytokine IFN-γ, IL-2, TNFα, IL-8, GM-CSF, IL-10, MIP-1α, MIP-1β, IL-17A, Fractalkine, ITAC, or combinations thereof at a level at a level within a log₂ fold change of 1 compared to an immune cell that has not experienced a cell engineering process.

In examples, the immune cell of the invention (e.g., the immune cell having the exogenous cargo) secretes the cytokine IFN-γ, IL-2, TNFα, IL-8, GM-CSF, IL-10, MIP-1α, MIP-1β, IL-17A, Fractalkine, ITAC, or combinations thereof at a level at a level about a log₂ fold change of −3 compared to an immune cell that has not experienced a cell engineering process. In other examples, the immune cell of the invention (e.g., the immune cell having the exogenous cargo) secretes the cytokine IFN-γ, IL-2, TNFα, IL-8, GM-CSF, IL-10, MIP-1α, MIP-1β, IL-17A, Fractalkine, ITAC, or combinations thereof at a level at a level about a log₂ fold change of −2 compared to an immune cell that has not experienced a cell engineering process. In other examples, the immune cell of the invention (e.g., the immune cell having the exogenous cargo) secretes the cytokine IFN-γ, IL-2, TNFα, IL-8, GM-CSF, IL-10, MIP-1α, MIP-1β, IL-17A, Fractalkine, ITAC, or combinations thereof at a level at a level about a log₂ fold change of −1 compared to an immune cell that has not experienced a cell engineering process.

Also provided herein is a method of delivering a cargo (or an “exogenous cargo”) across a plasma membrane of a non-adherent immune cell. Accordingly, the method includes providing a population of non-adherent cells and contacting the population of cells with a volume of an isotonic aqueous solution, the aqueous solution including the payload and an alcohol at greater than 0.2 percent (v/v) concentration, wherein an immune function of the non-adherent immune cell comprises a phenotype of a cell that has not experienced a cell engineering step, wherein the immune function is selected from (i) cytokine release; (ii) gene expression; and (iii) metabolic rate.

For example, the alcohol concentration is about 0.2 percent (v/v) concentration or greater, or the alcohol is about 0.5 percent (v/v) concentration or greater, or the alcohol is about 2 percent (v/v) or greater concentration. Alternatively, the alcohol concentration 5 percent (v/v) or greater. In other examples, the alcohol is 10 percent (v/v) or greater concentration.

For example, the alcohol comprises ethanol, e.g., 10% ethanol or greater. In some examples, the aqueous solution comprises between 20-30% ethanol, e.g., 27% ethanol.

Electroporation (a cell engineering process), for example, includes an intracellular delivery method where an electrical field is applied to cells to increase the cell membrane permeability (also called electrotransfer). As used herein, the term “cell engineering process” may refer to any known transfection method for intracellular delivery, including the SOLUPORE™ delivery method, membrane-disrupting methods (electroporation, sonoporation, magnetotection, optoperation), or carrier-based methods (lipid nanoparticles). Exemplary forms of electroporation include bulk electroporation and flow through electroporation. Suppliers and instrumentation for electroporation include Maxcyte, Lonza—Nucleofector, Cellectis—Pulse Agile, BioRad—Gene Pulser, Thermofisher—Neon, or Celetrix—Nanopulser.

Provided herein are methods for delivering a payload (or an “exogenous cargo”) across a plasma membrane of a non-adherent cell. The method includes providing a population of non-adherent cells; and contacting the population of cells with a volume of an isotonic aqueous solution, the aqueous solution including the payload and an alcohol at greater than 0.1, 0.5, 1, 2, 2.5, 5 percent (v/v) concentration or greater percent.

In examples, the aqueous solution includes alcohol, and the alcohol may include ethanol. In other examples, the aqueous solution comprises greater than 10% ethanol, between 20-30% ethanol, or about 27% ethanol. In examples, the aqueous solution comprises between 12.5-500 mM potassium chloride (KCl), or about 106 mM KCl.

The aqueous solution for delivering the exogenous cargo to cells comprises a salt, e.g., potassium chloride (KCl) in between 12.5-500 mM. For example, the solution is isotonic with respect to the cytoplasm of a mammalian cell such as a human T cell. Such an exemplary isotonic delivery solution 106 mM KCl.

In other examples, the aqueous solution can include an ethanol concentration of 5 to 30% (e.g., 0.2% to 30%). The aqueous solution can include one or more of 75 to 98% H₂O, 2 to 45% ethanol, 6 to 91 mM sucrose, 2 to 500 mM KCl, 2 to 35 mM ammonium acetate, and 1 to 14 mM (4-(2-hydroxyethyl)-1-piperazineethanesulfonic acid) (HEPES). For example, the delivery solution contains 106 mM KCl and 27% ethanol. For example, the delivery solution contains 106 mM KCl and 10% ethanol. For example, the delivery solution contains 106 mM KCl and 5% ethanol. For example, the delivery solution contains 106 mM KCl and 2% ethanol.

Exemplary non-adherent/suspension cells include primary hematopoictic stem cell (HSC), T cells (e.g., CD3+ cells, CD4+ cells, CD8+ cells), natural killer (NK) cells, cytokine-induced killer (CIK) cells, human cord blood CD34+ cells, B cells, or cell lines such as Jurkat T cell line. The of non-adherent cells can be substantially confluent, such as greater than 75 percent confluent. Confluency of cells refers to cells in contact with one another on a surface. For example, it can be expressed as an estimated (or counted) percentage, e.g., 10% confluency means that 10% of the surface, e.g., of a tissue culture vessel, is covered with cells, 100% means that it is entirely covered. For example non-adherent cells can be spun down, pulled down by a vacuum, or tissue culture medium aspiration off the top of the cell population, or removed by aspiration or vacuum removal from the bottom of the vessel. The cells can form a monolayer of cells. For example, for the cells are 10, 25, 50, 75, 90, 95, or 100% confluent.

In embodiments, the immune cell is not activated prior to cargo delivery. In other examples, the immune cell has not been contacted with a ligand of CD3, CD28, or a combination thereof, prior to contacting the immune cell with the exogenous cargo.

In embodiments, the non-adherent cell comprises a peripheral blood mononuclear cell. In examples, the non-adherent cell comprises an immune cell, for example a T lymphocyte, and the immune cell is unstimulated, for example by either CD3 or CD28, or any combination thereof. In other examples, the non-adherent cell comprises an immune cell, for example a T lymphocyte.

In embodiments, the immune cell comprises an unstimulated immune cell. For example, the immune cell is not stimulated with a ligand of CD3, CD28, or a combination thereof. Put another way, the immune cell is not contacted with a CD3 or CD28 ligand, for example, an antibody or antibody fragment that binds to CD3, CD28, or both. In examples, the population of non-adherent cells comprises a monolayer. For example, the monolayer is contacted with a spray of said aqueous solution.

The method involves delivering the exogenous cargo in the delivery solution to a population of non-adherent cells comprising a monolayer. For example, the monolayer is contacted with a spray of aqueous delivery solution. The method delivers the payload/cargo (compound or composition) into the cytoplasm of the cell and wherein the population of cells comprises a greater percent viability compared to delivery of the payload by electroporation or nucleofection, a significant advantage of the Soluporation system.

In certain embodiments, the monolayer of non-adherent/suspension cells resides on a membrane filter. In some embodiments, the membrane filter is vibrated following contacting the cell monolayer with a spray of the delivery solution. The membrane filter may be vibrated or agitated before, during, and/or after spraying the cells with the delivery solution.

The volume of solution to be delivered to the cells is a plurality of units, e.g., a spray, e.g., a plurality of droplets on aqueous particles. The volume is described relative to an individual cell or relative to the exposed surface area of a confluent or substantially confluent (e.g., at least 75%, at least 80% confluent, e.g., 85%, 90%, 95%, 97%, 98%, 100%) cell population. For example, the volume can be between 6.0×10⁻⁷ microliter per cell and 7.4×10⁻⁴ microliter per cell. The volume is between 4.9×10⁻⁶ microliter per cell and 2.2×10⁻³ microliter per cell. The volume can be between 9.3×10⁻⁶ microliter per cell and 2.8×10⁻⁵ microliter per cell. The volume can be about 1.9×10-5 microliters per cell, and about is within 10 percent. The volume is between 6.0×10⁻⁷ microliter per cell and 2.2×10⁻³ microliter per cell. The volume can be between 2.6×10⁻⁹ microliter per square micrometer of exposed surface area and 1.1×10-6 microliter per square micrometer of exposed surface area. The volume can be between 5.3×10-8 microliter per square micrometer of exposed surface area and 1.6×10-7 microliter per square micrometer of exposed surface area. The volume can be about 1.1×10-7 microliter per square micrometer of exposed surface area.

Throughout the specification the term “about” can be within 10% of the provided amount or other metric.

Confluency of cells refers to cells in contact with one another on a surface. For example, it can be expressed as an estimated (or counted) percentage, e.g., 10% confluency means that 10% of the surface, e.g., of a tissue culture vessel, is covered with cells, 100% means that it is entirely covered. For example, adherent cells grow two dimensionally on the surface of a tissue culture well, plate or flask. Non-adherent cells can be spun down, pulled down by a vacuum, or tissue culture medium aspiration off the top of the cell population, or removed by aspiration or vacuum removal from the bottom of the vessel.

The payload (exogenous cargo) can include a small chemical molecule, a peptide or protein, or a nucleic acid. The small chemical molecule can be less than 1,000 Da. The chemical molecule can include MitoTrackerg Red CMXRos, propidium iodide, methotrexate, and/or DAPI (4′,6-diamidino-2-phenylindole). The peptide can be about 5,000 Da. The peptide can include ecallantide under trade name Kalbitor, is a 60 amino acid polypeptide for the treatment of hereditary angioedema and in prevention of blood loss in cardiothoracic surgery), Liraglutide (marketed as the brand name Victoza, is used for the treatment of type II diabetes, and Saxenda or the treatment of obesity), and Icatibant (trade name Firazyer, a peptidamimetic for the treatment of acute attacks of hereditary angioedema). The small-interfering ribonucleic acid (siRNA) molecule can be about 20-25 base pairs in length, or can be about 10,000-15,000 Da. The siRNA molecule can reduces the expression of any gene product, e.g., knockdown of gene expression of clinically relevant target genes or of model genes, e.g., glyceraldehyde-3phosphate dehydrogenase (GAPDH) siRNA, GAPDH siRNA-FITC cyclophilin B siRNA, and/or laminsi RNA. Protein therapeutics can include peptides, enzymes, structural proteins, receptors, cellular proteins, or circulating proteins, or fragments thereof. The protein or polypeptide be about 100-500,000 Da, e.g., 1,000-150,000 Da. The protein can include any therapeutic, diagnostic, or research protein or peptide, e.g., beta-lactoglobulin, ovalbumin, bovine serum albumin (BSA), and/or horseradish peroxidase. In other examples, the protein can include a cancer-specific apoptotic protein, e.g., Tumor necrosis factor-related apoptosis inducing protein (TRAIL).

An antibody is generally be about 150,000 Da in molecular mass. The antibody can include an anti-actin antibody, an anti-GAPDH antibody, an anti-Src antibody, an anti-Myc ab, and/or an anti-Raf antibody. The antibody can include a green fluorescent protein (GFP) plasmid, a GLuc plasmid and, and a BATEM plasmid. The DNA molecule can be greater than 5,000,000 Da. In some examples, the antibody can be a murine-derived monoclonal antibody, e.g., ibritumomab tiuxetin, muromomab-CD3, tositumomab, a human antibody, or a humanized mouse (or other species of origin) antibody. In other examples, the antibody can be a chimeric monoclonal antibody, e.g., abciximab, basiliximab, cetuximab, infliximab, or rituximab. In still other examples, the antibody can be a humanized monoclonal antibody, e.g., alemtuzamab, bevacizumab, certolizumab pegol, daclizumab, gentuzumab ozogamicin, trastuzumab, tocilizumab, ipilimumamb, or panitumumab. The antibody can comprise an antibody fragment, e.g., abatecept, aflibercept, alefacept, or etanercept. The invention encompasses not only an intact monoclonal antibody, but also an immunologically-active antibody fragment, e.g. a Fab or (Fab)2 fragment; an engineered single chain Fv molecule; or a chimeric molecule, e.g., an antibody which contains the binding specificity of one antibody, e.g., of murine origin, and the remaining portions of another antibody, e.g., of human origin.

The payload (or “exogenous cargo”) can include a therapeutic agent. A therapeutic agent, e.g., a drug, or an active agent, can mean any compound useful for therapeutic or diagnostic purposes, the term can be understood to mean any compound that is administered to a patient for the treatment of a condition. Accordingly, a therapeutic agent can include, proteins, peptides, antibodies, antibody fragments, and small molecules. Therapeutic agents described in U.S. Pat. No. 7,667,004 (incorporated herein by reference) can be used in the methods described herein. The therapeutic agent can include at least one of cisplatin, aspirin, statins (e.g., pitavastatin, atorvastatin, lovastatin, pravastatin, rosuvastatin, simvastatin, promazine IICl, chloropromazine HO, thioridazine HCl, Polymyxin B sulfate, chloroxine, benfluorex HCl and phenazopyridine HCl), and fluoxetine. The payload can include a diagnostic agent. The diagnostic agent can include a detectable label or marker such as at least one of methylene blue, patent blue V, and Indocyanine green. The payload can include a fluorescent molecule. The payload can include a detectable nanoparticle. The nanoparticle can include a quantum dot.

The payload (“exogenous cargo”) includes an alcohol. By the term “an alcohol” is meant a polyatomic organic compound including a hydroxyl (—OH) functional group attached to at least one carbon atom. The alcohol may be a monohydric alcohol and may include at least one carbon atom, for example methanol. The alcohol may include at least two carbon atoms (e.g. ethanol). In other aspects, the alcohol comprises at least three carbons (e.g. isopropyl alcohol). The alcohol may include at least four carbon atoms (e.g., butanol), or at least seven carbon atoms (e.g., benzyl alcohol). The example payload may include no more than 50% (v/v) of the alcohol, more preferably, the payload comprises 2-45% (v/v) of the alcohol, 5-40% of the alcohol, and 10-40% of the alcohol. The payload may include 20-30% (v/v) of the alcohol.

Most preferably, the payload delivery solution includes 25% (v/v) of the alcohol. Alternatively, the payload can include 2-8% (v/v) of the alcohol, or 2% of the alcohol. The alcohol may include ethanol and the payload comprises 5, 10, 20, 25, 30, and up to 400/0 or 50% (v/v) of ethanol, e.g., 27%. Example methods may include methanol as the alcohol, and the payload may include 5, 10, 20, 25, 30, or 40% (v/v) of the methanol. The payload may include 2-45% (v/v) of methanol, 20-30% (v/v), or 25% (v/v) methanol. Preferably, the payload includes 20-30% (v/v) of methanol. Further alternatively, the alcohol is butanol and the payload comprises 2, 4, or 8% (v/v) of the butanol.

In some aspects of the present subject matter, the payload is in an isotonic solution or buffer.

According to the present subject matter, the payload may include at least one salt. The salt may be selected from NaCl, KCl, Na₂HPO₄, C₂H₃O₂NH₄ and KH₂PO₄. For example, KCl concentration ranges from 2 mM to 500 mM. In some preferred embodiments, the concentration is greater than 100 mM, e.g., 106 mM. According to example methods of the present subject matter, the payload may include a sugar (e.g., a sucrose, or a disaccharide). According to example methods, the payload comprises less than 121 mM sugar, 6-91 mM, or 26-39 mM sugar. Still further, the payload includes 32 mM sugar (e.g., sucrose). Optionally, the sugar is sucrose and the payload comprises 6.4, 12.8, 19.2, 25.6, 32, 64, 76.8, or 89.6 mM sucrose.

In embodiments, the methods for delivering an exogenous cargo across the plasma membrane of the immune cell further comprise delivering at least two exogenous cargos (or “two payloads”). The exogenous cargo comprises a nucleic acid, a small molecule, a protein, a polypeptide, or a combination thereof. For example, the nucleic acid comprises messenger ribonucleic acid (mRNA), small interfering RNA (siRNA), short hairpin RNA (shRNA), deoxyribonucleic acid (DNA), or any combination thereof. In examples, the immune cells comprises two exogenous cargos, 3 exogenous cargos, 4, 5, 6, 7, 8, 9, or 10 exogenous cargos.

In embodiments, the at least two exogenous cargos are simultaneously delivered, meaning the two exogenous cargos are delivered at the same time (e.g., dual delivery). For example the immune cell of the invention (comprising an exogenous cargo), may be manipulated to comprise a second exogenous cargo. As used herein, the term “manipulated” may refer to any known transfection method for intracellular delivery, including the SOLUPORE™ delivery method, membrane-disrupting methods (electroporation, sonoporation, magnetotection, optoperation), or carrier-based methods (lipid nanoparticles).

In embodiments, the at least two exogenous cargos are sequentially delivered. For example, sequentially delivered may refer to delivery of one exogenous cargo, followed by delivery of a second, third, or fourth exogenous cargo. For example the immune cell of the invention (comprising an exogenous cargo), may then further be manipulated to comprise a second exogenous cargo. As used herein, the term “manipulated” may refer to any known transfection method for intracellular delivery, including the SOLUPORE™ delivery method, membrane-disrupting methods (electroporation, sonoporation, magnetotection, optoperation), or carrier-based methods (lipid nanoparticles). Electroporation, for example, includes an intracellular delivery method where an electrical field is applied to cells to increase the cell membrane permeability (also called electrotransfer).

In other aspects, provided herein is a method of delivering a an exogenous cargo across a plasma membrane of a non-adherent cell, the method comprising the steps of providing a population of non-adherent cells and using at least two intracellular delivery methods selected from (i) contacting the population of cells with a volume of an isotonic aqueous solution, the aqueous solution including the exogenous cargo and an alcohol at greater than 0.5 percent (v/v) concentration, (ii) viral transduction, (iii), electroporation or (iv) nucleofection, and thereby delivering the two exogenous cargos to the immune cell. For example, aqueous solution comprises alcohol, and the alcohol comprises ethanol. The concentration of alcohol in the aqueous solution is greater than 0.2 percent (v/v) concentration, or greater than 0.5 percent (v/v) concentration, or greater than 2 percent (v/v) concentration, or greater than 5 percent (v/v) concentration, or greater than 10 percent (v/v) concentration. In some examples, the aqueous solution comprises between 20-30% ethanol, e.g., 27% ethanol.

In embodiments, the intracellular delivery methods comprise contacting the population of cells with a volume of an isotonic aqueous solution, the aqueous solution including the exogenous cargo and an alcohol at greater than 0.5 percent (v/v) concentration followed by viral transduction. In other examples, the intracellular delivery methods comprise viral transduction followed by contacting the population of cells with a volume of an isotonic aqueous solution, the aqueous solution including the exogenous cargo and an alcohol at greater than 0.5 percent (v/v) concentration.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### DESCRIPTION OF THE DRAWINGS

FIGS. 1A-1D are bar graphs showing efficient engineering of T cells with the SOLUPORE™ delivery method. Model cargo, GFP (green fluorescent protein) expression and cell viability at 24 hr following GFP mRNA delivery for (FIG. 1A) PBMC (peripheral blood mononuclear cells)-initiated T cells and (FIG. 1B) CD3+(cluster of differentiation 3) purified T cells is shown. FIG. 1C is a bar graph showing CD3 expression and cell viability at day 2 post-delivery of TRAC (T cell receptor alpha constant) RNPs (ribonucleoprotein). FIG. 1D is a bar graph showing PD-1 (programmed death protein 1) INDEL (insertion or deletion of bases) efficiency, quantified by Sanger sequencing and TIDE (Tracking of Indels by Decomposition) analysis, and cell viability in cells harvested at day 4 post-delivery of PDCD1 (Programmed cell death protein 1) RNPs. All studies used cells from 3 donors, n=2.

FIGS. 2A-2D are graphs showing that the SOLUPORE™ delivery method enabled dual and sequential cargo delivery for multiple modifications. FIG. 2A are bar graphs showing the co-expression of CD19 (cluster of differentiation 19) CAR (chimeric antigen receptor) and GFP by flow cytometry and cell viability at 24 hr following dual delivery of corresponding mRNAs, n=3. FIG. 2B is a series of representative flow cytometry plots, showing data from one donor, showing expression of CAR only, GFP only and the population of cells that express both CAR and GFP. FIG. 2C is a series of bar graphs showing the expression of CD19 CAR, CD3 knockdown by flow cytometry and cell viability on day 3 following sequential delivery of TRAC RNP on day 0 and CD19 CAR mRNA on day 2, n=3. FIG. 2D are representative flow plots showing data from one donor, showing expression of CD3 only, CAR only and the expression of CD3 and CAR in the population.

FIGS. 3A-3C are depictions of data that show that the comparison of intracellular delivery methods revealed minimal perturbation of cytokine release and immune gene expression with the SOLUPORE™ delivery method. FIG. 3A is a series of line graphs showing cytokine release from activated T cells following the SOLUPORE™ delivery method compared to electroporation delivery of GFP mRNA, measured by Luminex multiplex assay, 5 donors with 2 technical repeats included for each donor. FIG. 3B is a series of Volcano plots where each dot represents a gene and its position in the plot represents the extent to which it has been up or downregulated compared to control cells. The Volcano plots show results from a study of unactivated T cells (Study 1) from 3 donors were mock-transfected, RNA was harvested 6 h or 24 h post-treatment and gene expression was compared with untreated control cells using the Nanostring CAR-T Characterisation panel. The Volcano plots showed the fold change and p value of individual genes at 6 h and 24 h after transfection, compared to untreated control cells. FIG. 3C is a filtered heatmap that indicates gene expression altered by more than 1 log 2 fold (>2 fold) with a statistical significance of p<0.05, showing only those genes that were changed in at least one of the groups. Green, red and black (shown in shading and arrows) represent down-regulated, up-regulated and not changed, respectively. See also Tables 7, 8 and 9.

FIGS. 4A and 4B are graphs showing that intracellular deliver methods differentially impact affect T cell proliferation rate and in vivo engraftment. FIG. 4A is a line graph showing that proliferation of T cells following GFP mRNA delivery using either the SOLUPORE™ delivery method or electroporation, N=5 donors each with n=5 technical repeats. FIG. 4B is a graph showing the engraftment of human CD45+(cluster of differentiation) cells in spleen of NOD-scid IL-2Rγnull (non-obese diabetic (NOD) severe combined immune deficiency (SCID) mice at 28 days post-injection.

FIG. 5A is a graph showing CAR expression in T cells at 24 hr post-delivery and in vitro killing of RAJI tumor cells as measured by impedance assay from 3 different donors. FIG. 5B is a diagram of a schematic protocol that was used to assess the ability of CD19 CAR T to kill RAJI cells in an established model. 2.5×10⁵ luciferase-expressing RAJI cells were injected into NOD-scid IL-2Rγnull mice followed 3 days later by 1×10⁶, 2×10⁶ or 4×10⁶ cells treated either by SOLUPORE™ or electroporation, n=10 mice per group. Bioluminescence was measured on day 15 at which point animals were euthanized. FIG. 5C is a photographic image depicting bioluminescence imaging at day 15. FIG. 5D is a series of dot plot graphs showing that human T cells were detected in the blood of mice by flow cytometry analysis of human CD3 expression at day 15. FIG. 5E is a bar graph showing that RAJI tumor cells were detected in the blood of mice by flow cytometry analysis of human CD20 (cluster of differentiation 20) expression at day 15. FIGS. 5A-5E demonstrate that CD19 CAR-T cells showed effective cytotoxicity in vitro and in vivo. CAR-T cells were generated with the SOLUPORE™ delivery method or electroporation-mediated delivery of CD19 CAR mRNA.

FIGS. 6A-6I are line graphs showing cytokine release from activated T cells following the SOLUPORE™ delivery method or electroporation delivery of GFP mRNA, measured by Luminex multiplex assay, 5 donors were used with 2 technical repeats included for each donor. FIG. 6A is a graph of showing release of IFN (interferon)-gamma. FIG. 6B is a graph showing release of IL-10 (interleukin 10). FIG. 6C is a graph showing release of TNF-α (tumor necrosis factor alpha). FIG. 6D is a graph showing release of GM CSF (Granulocyte-macrophage colony-stimulating factor). FIG. 6E is a graph showing release of MIP-1α(macrophage inflammatory protein 1a). FIG. 6F is a graph showing release of MIP-1b (macrophage inflammatory protein 1b). FIG. 6G is a graph showing release of ITAC (Interferon—inducible T Cell Alpha Chemoattractant). FIG. 6H is a graph showing release of fractalkine. FIG. 6I is a graph showing release of II-17A (interleukin 17A). In summary, the cytokine release data in FIG. 6A-6I indicated that the SOLUPORE™ delivery method causes minimal stress to T cells, as evidenced by no difference in cytokine release compared with untreated control cells. In contrast, the electroporation process caused release of IL-2 and IL-8 from T cells.

FIG. 7 is an image of an unfiltered heatmap which indicates where gene expression was altered by more than 1 log 2 fold (>2 fold) with a statistical significance of p<0.05, including all genes analysed. Green, red and black (shown in shading and arrows) represent down-regulated, up-regulated and not changed, respectively.

FIG. 8 is an image showing a pathway analysis from an immune-related gene profiling study. Blue is a downregulation of a gene and red is upregulation of a gene 6 h post-transfection. A beige or light shade of blue/red indicates gene expression more similar to UT with no change being beige. The closer the colour to the far edges of the colour key the higher fold change of gene expression with deep blue being −15 and deep red being 15 z-activation score. Gene names are shown on the y axis and the x-axis recites “Treatment Mock N(F115) vs. UT” and “Treatment Mock vs. UT,” from left to right.

FIGS. 9A-9C are bar graphs showing results of the area under the curve (AEC) in in vitro RAJI cells killing assay calculated for each donor (FIG. 9A—Donor 1; FIG. 9B—Donor 2, and FIG. 9C—Donor 3).

FIG. 10 is a diagram showing the AP-1 (Activator Protein 1) signaling pathway.

FIG. 11 is a table showing of AP-1 related genes from Study 1 and Study 2 (see Example 4). The cells in both Study 1 and 2 were unstimulated cells. Study 2 was a repeat that included only 24 hr analysis and the EO-115 electroporation method.

FIG. 12 is a depiction of log 2 fold change vs linear change. A comparison or calculator of gene expression differences as expressed by log 2 fold change or linear fold change in shown in the table. The increase or decrease in the gene or protein expression may be expressed as fold-difference or log-difference. The term “log base 2” or log₂ was used to normalize the results along an axis with equal values for upregulated and downregulated genes. An exemplary calculation is shown below:

gene A treated vs control=7.0 (overexpressed);

gene B control vs treated=7.0 or treated vs control=0.142 (underexpressed).

Both are overexpressed or underexpressed with the same intensity however, a linear scale would not reflect this change. Alternatively, gene A is 7.0 fold up and gene 2 is 0.142 down regulated. When expressed in the format of log₂, gene A is 2.81 fold upregulated and gene B is −2.81 fold downregulated.

FIG. 13 depict images of surface programmed cell death protein 1 (PD-1) staining performed on activated T cells, either following soluporation or nucleofection.

FIG. 14 are images depicting surface cluster differentiation 69 (CD69) staining performed on activated T cells, either following soluporation or nucleofection.

FIG. 15 depicts a bar graph showing activated human T cells that were either soluporated or nucleofected (electroporation protocol EO115) with or without mRNA-GFP and supernatants were harvested 6 h post transfection. The ChromaDazzle lactate assay was carried out. L-lactate concentration was extrapolated from a standard curve using Microsoft Excel and data was presented as fold change relative to control. 5 donors, n=2.

FIGS. 16A and 16B depicts a series of illustrations of calculations made from Seahorse traces. Calculation of spare respiratory capacity (SRC), maximal respiration and rate of basal oxidative phosphorylation (OxPhos) from oxygen consumption rate (OCR) trace (FIG. 16A) and glycolytic reserve, glycolytic capacity and basal rate of glycolysis from extracellular acidification rate (ECAR trace) (FIG. 16B).

FIG. 17 depicts a series of line graphs showing activated human T cells that were either Soluporated or nucleofected (EO115) with mRNA-GFP or in the absence of cargo (mock). Cells were harvested and a Seahorse assay was conducted, the above. Shows the OCR and ECAR traces (n=1).

FIG. 18 depicts a series of bar graphs showing glycolysis, OxPhos, Glycolytic capacity and maximal respiration of soluporated or nucleofected T cells. This data depicts metabolic activity of the cells approximately 18 hours post transfection.

FIG. 19A depicts images showing that GFP expression on CAR positive cell was analysed at 24 hours post-transfection by flow cytometry. FIG. 19B is a bar graph showing that the viability was assessed using NC-3000 24 hours post-transfection. n=1 in 3 donors. UT=untreated control. PBMC-initiated T cells were transduced with CD19 CAR lentiviral vector on day 3 post-activation (LV-CAR). Cells were harvested 24 hours post-viral delivery and subsequently transfected with GFP mRNA using SOLUPORE™ technology.

### DETAILED DESCRIPTION

Provided herein are, cell engineering technologies that enable next generation cell therapy products which require complex modifications and high levels of cell functionality. Non-viral engineering technologies address limitations associated with viral vectors. Electroporation is the most widely used non-viral modality but concerns about its effects on cell functionality led to the exploration of alternative approaches. As described herein, the SOLUPORE™ delivery method is a non-viral means of simply, rapidly and efficiently delivering cargos to primary immune cells, while retaining cell viability and functionality.

### Challenges of Viral Delivery Methods

Safe, flexible and efficient intracellular delivery of exogenous material is a critical requirement for a number of cell engineering applications, examples of which include the treatment of haematological malignancies and disorders, wherein immune cells are modified ex vivo to replace, correct or insert targeted genes. Whilst viral transduction has been most commonly used for genetic manipulation, its limitations are well-known.

The timeline from initiation of production to batch release of Good Manufacturing Practice (GMP) vectors for cellular therapies, including acquisition of plasmid DNA for transfection, can be lengthy and costly. Challenges around the scalability of vector production and the associated costs have driven an interest in the development of non-viral alternatives. Viral delivery systems are also susceptible to vector-mediated genotoxicity, such as random insertions disrupting normal genes, accidental oncogene activation or insertional mutagenesis leading to adverse immunogenicity and severe side effects. Aside from the biosafety concerns, constraints on the cargo packaging capacity of viral vectors have also motivated the development of intracellular delivery methods which can be used to deliver a broader range of bioactive constructs. A broader multiplexing potential combined with a flexibility to accommodate accelerated manufacturing timelines and changes between cell types and sizes whilst avoiding the side effects associated with viral vectors are attractive attributes in any one intracellular delivery method making them safer and more economical.

### CAR T Cell Therapy

Autologous CAR T cell therapy has shown unprecedented efficacy as well as durable responses in cohorts of replapsed or refractory cancer patients with select liquid tumors resulting in two CAR T product approvals to date. The proof of concept generated with these cell products is driving research, development and commercial activity in the ex vivo cell therapy field. The success of these ‘living’ drugs was achieved in spite of complex manufacturing and logistical processes that have created a new paradigm for drug manufacture. Engineering of the early breakthrough products was enabled by viral vectors and while this delivery modality remains important, issues with availability, complexity, cost, safety and efficiency mean that advanced gene transfer technologies are needed for the next generation of therapies. The invention provides engineered cell populations containing exogenous cargo molecules to address the drawbacks and challenges of previous approaches to introducing nucleic acids and other molecules into cells. If the promise of engineered cell therapy successes is to be realised in patients with alternative and earlier stage liquid tumors and patients with solid tumors, the key focus areas must include optimization of cell therapies for liquid tumors, acceleration of the innovation cycle time to enable success in solid tumors and transformation of manufacturing processes. The virus-free protocol described here plays a key role in all of these aspects, ultimately improving patient access. The method described herein does not rely on virus or subjecting cells to an electrical current to mediate delivery of exogenous molecules into cells. The method described herein does not rely on lipid nano-particles to mediate delivery of exogenous molecules into cells.

### Non-Viral Delivery

Unlike conventional viral transduction, non-viral alternatives can deliver a broader range of constructs into more diverse cell types, whilst circumventing the intensive biosafety and regulatory requirements for vector production for cellular therapies.

Intracellular delivery can be facilitated by a range of techniques, broadly classified into two main categories of either membrane-disrupting methods or carrier-based.

Intracellular delivery methods can be broadly classified into two main categories, namely physical/mechanical methods such as electroporation, sonoporation, magnetotection, optoperation, gene gun, microinjection, cell constriction/squeezing, and non-viral vectors such as lipid nano-particles. Whilst electroporation platforms enable efficient delivery of cargo, some challenges exist such as the loss of proliferative capacity, decreased potency, sustained intracellular calcium levels.

Chemical vectors such as cationic polymers and lipids can deliver genetic material into cells without provoking a significant immune response, however, to date, their efficiency is not comparable to viral counterparts.

Membrane-disrupting modalities, the SOLUPORE™ delivery method, have the potential to increase processing throughput, reduce manufacturing time, minimising processing steps, but yet produce a highly functional and potent cell.

Some physical transfection methods can impact cell health leading to deleterious effects on their proliferative capacity accompanied by changes in their gene expression profiles.

Good in vitro proliferation and effector function correlates with improved antitumor function in vivo, therefore, the SOLUPORE™ intracellular delivery method allows for transfection of a diverse array of cargo to multiple cell types whilst minimally perturbing normal cell function.

To address the consequences associated with perturbing normal cell function, a SOLUPORE™ delivery method was developed. The SOLUPORE™ delivery method is a non-viral, non-electrical (does not utilize application of an electrical current to cells) technology that allows for transient permeabilization of the cell membrane to achieve rapid intracellular delivery of cargos with varying composition, properties and size such as macromolecules and nucleic acid. As demonstrated, the SOLUPORE™ delivery method successfully facilitated the delivery of gene-editing tools such as CRISPR/Cas9 and mRNA to primary human immune cells, including human T cells, without negatively impacting cell function.

Moreover, the SOLUPORE™ delivery platform was developed as an advanced technology aimed at addressing development and manufacturing needs of the cell therapy field. The technology is non-viral meaning that many issues associated with viral vectors such availability, safety, process complexity and associated costs are less of a concern. Continuing advances in gene engineering tools also mean that genome targeting is now possible with non-viral approaches.

As described herein, the delivery efficiency of the SOLUPORE™ delivery method was evaluated with a wide range of cargo types as well as its flexibility in addressing T cell populations that have been cultured using diverse protocols. While other non-viral instruments such as electroporators frequently require cell-specific programs and buffers, the same SOLUPORE™ delivery method programs and buffers can be used for a wide range of cell types with cell density being the main parameter that is varied. A table of cell seeding densities is provided below (predicted range was calculated using average cell size). A high level of consistency is seen in the results achieved making for predictable processes.

TABLE 18
Cell seeding densities

| | Optimal seeding | Predicted |
|---|---|---|
| Cell type | density range | range |
| Bead-activated T cells | 15-20e6 | |
| PBMC-initiated T cells | 6-10e6 | |
| NK cells | 10-15e6 | |
| Dendritic cells | | 5-14e6 |
| Macrophages | | 5-15e6 |
| HSC | | 10-20e6 |
| Plasma cells | | 4-8e6 |

The ability of the platform to support dual and sequential gene edits without compromising cell viability is an important feature. If targeting and efficacy are to be enhanced in autologous cell therapies for both liquid and solid tumors, cells will require multiple modifications using steps that are aligned with manufacturing processes. This may involve multiplex or sequential engineering steps. Similar demands apply to allogeneic approaches where cell rejection and GvHD issues mean that complex editing is likely to be required. Limitations in viral vector capacity and electroporation toxicity mean that these modalities may be unsuitable for certain complex engineering regimes. Moreover, the long lead time required to design and generate even research grade viral vectors means that timelines may be longer than desired at the development stage. This is of particular concern in relation to progressing approaches for solid tumors where targeting and efficacy challenges mean that large numbers of candidate target antigens and cell potency enhancements will need to be tested. It will be necessary to evaluate a myriad of cell compositions in a rapid, high-throughput fashion that is likely to be highly constrained if wholly reliant on viral vectors. Thus the SOLUPORE™ delivery method addresses these concerns and is compatible with optimization of CAR T for liquid tumors and the acceleration of the innovation cycle time required for impactful progress in tackling solid tumors.

### Cells Processed Using the SOLUPORE™ Delivery Method Retain Critical Immune Function and Minimize Exhaustion

Cell functionality must be maintained if cell engineering is to be useful. Thus, there is interest in the field in developing alternative non-viral delivery methods that can be efficient whilst also being gentle on cells. The studies reported herein demonstrate that the SOLUPORE™ delivery method has a minimal impact on protein and gene expression in T cells and, importantly, biological attributes such as proliferation and gene expression profiles are preserved. Moreover using the SOLUPORE™ delivery method, CAR T cells killed cells both in vitro and in vivo, thus demonstrating the functionality of these cells.

While electroporation is the most widely used non-viral method for cargo delivery, non-specific changes in protein and gene expression and reduced anti-tumor efficacy have been observed previously in T cells engineered by this method. The SOLUPORE™ delivery method altered the expression of only a small number of immune-related genes. Of the 10 genes identified in the 6 hr group using the SOLUPORE™ delivery method (at 6 hours post procedure), 8 were common with the electroporation 6 hr group suggesting that these may be genes associated with breaching the cell membrane or other aspects common to the two delivery methods.

The finding that electroporation dramatically affects gene expression in T cells is consistent with a study of increased levels of intracellular calcium and increased transcriptional activity in electroporated T cells in the absence of exogenous stimuli (see, Zhang M, et a. J Immunol Methods 2014; 408: 123-131, incorporated herein by reference in its entirety). Calcium release from the endoplasmic reticulum leads to activation of the transcription factor NFAT (Nuclear factor of activated T-cells), one of the central regulators of exhaustion.

The observation that increased expression of genes involved in AP-1 (activator protein 1) signalling occurred in electroporated cells suggested that transcription factors such as AP-1 and NFAT may play roles in cell stress responses to electroporation. The results described herein support studies showing electroporation-induced perturbation of these key pathways contribute to cell exhaustion, which renders the cells less suitable for mammalian cell therapy. In contrast, the SOLUPORE™ delivery method causes minimal perturbation of these pathways with profiles of treated cells remaining close to those of control cells.

Electroporation, including nucleofection, leads to reduced cell proliferation, which is suggested to be caused by activation of DNA damage response pathways. This is a concern for gene editing approaches and it is unclear how these effects of electroporation ultimately impact the potency of cell therapy products. While electroporated CAR T cells performed similar to cells using the SOLUPORE™ delivery method in the 12 day RAJI tumor mouse model, electroporated cells failed to engraft as expected in the 30 day in vivo engraftment model indicating that the fitness of the cells was negatively impacted.

There is also increasing interest in the possibility of engineering unactivated T cells to reduce cell exhaustion. According to the invention, unactivated cells are engineered and subsequently expanded. This approach has the added advantage of requiring substantially less cargo. The findings herein, and by others demonstrated that electroporation induced perturbations (indicative of cell exhaustion) in unactivated T cells at the transcriptional level, and thus makes nucleofection/electroporation a less desirable approach for this application. In contrast, the SOLUPORE™ delivery method has the potential to enable engineering of these cells.

### AP-1 Signaling and T Cell Exhaustion

Activator protein 1 (AP-1) is a transcription factor that regulates gene expression in response to a variety of stimuli, including cytokines, growth factors, stress, and bacterial and viral infections. AP-1 controls a number of cellular processes including differentiation, proliferation, and apoptosis.

The Activator protein-1 (AP-1), is a group of transcription factors consisted of four sub-families:

1) Jun (“v-jun avian sarcoma virus 17 oncogene homolog, jun oncogene” or “c-jun”), c-Jun (transcription factor AP1), JunB (Transcription factor jun-B), JunD (transcription factor jun-D isoform deltaJunD)),

2) Fos (c-Fos (proto-oncogene)—FosB (also known as FosB and G0/G1 switch regulatory protein 3 (G0S3)), Fra1 (Fos-related antigen 1 (FRA1)), Fra2 (Fos-related antigen 2 (FRA2)),

3) Maf (musculoaponeurotic fibrosarcoma)—(c-Maf (also known as proto-oncogene c-Maf or V-maf musculoaponeurotic fibrosarcoma oncogene homolog), MafB (also known as V-maf musculoaponeurotic fibrosarcoma oncogene homolog B), MafA (Transcription factor MafA), Mafg/f/k (bZip Maf transcription factor protein), Nrl (Neural retina-specific leucine zipper protein)), and

4) ATF-activating transcription factor (ATF2 (Activating transcription factor 2), LRF1/ATF3 (Cyclic AMP-dependent transcription factor ATF-3), BATF (Basic leucine zipper transcription factor, ATF-like), JDP1 (DnaJ (Hsp40) homolog, subfamily C), JDP2 (Jun dimerization protein 2)).

These AP-1 transcription factors regulate a wide range of cellular processes spanning from cell proliferation and survival to tumor transformation, differentiation and apoptosis. AP-1 transcription factors are homo- or hetero-dimmer forming proteins. Members of the AP-1 protein family differ markedly in their potential to transactivate AP-1 responsive genes and their ability to form dimmers. For example, the Fos sub-family cannot homodimerize, but they can form stable heterodimers with Jun members. The Fos and Jun proteins have high transactivation potential, whereas others like JunB, JunD, Fra-1 and Fra-2 are weaker. Early studies using murine fibroblasts, substantiate the antagonistic nature of some AP-1 members against others. For instance, cJun transcriptional activity is attenuated by JunB and this is due to differences in their activation domains. Nevertheless, the current viewpoint suggests that the differential expression of AP-1 components and the cell context of their interactions determines the complex functions of AP-1 transcription factor.

In T cells, AP-1 transcription factors are characterized by pleiotropic effects and a central role in different aspects of the immune system such as T-cell activation, Th differentiation, T-cell anergy and exhaustion. MAPK (MAP kinase) signaling cascade is important for regulating AP-1 transcriptional activation and DNA binding activity on a wide array of AP-1 target genes.

T-cell anergy is an unresponsive state of T-cells in which T-cells are activated in the absence of a positive costimulatory signal, while T-cell exhaustion is referred to the state of CD8⁺T cells that respond poorly because of prolonged antigen exposure during chronic viral infections or cancer. Some of the hallmarks of anergic T cells are the inhibition of proliferation and their inability to synthesize IL-2 in response to TCR (T cell receptor) engagement.

T cell exhaustion is characterized by high expression of inhibitory receptors and widespread transcriptional and epigenetic alterations but the mechanisms responsible for impaired function in exhausted T cells are unknown. Blockade of PD-1 (programmed death protein 1) can reinvigorate some exhausted T cells but does not restore function fully, and trials using PD-1 blockade in combination with CAR T cells have not demonstrated efficacy. Models in which healthy T cells are driven to exhaustion by the expression of a tonically signalling CAR, exhausted human T cells have demonstrated widespread epigenomic dysregulation of AP-1 transcription factor-binding motifs and increased expression of the bZIP and IRF transcription factors that have been implicated in the regulation of exhaustion-related genes. See, Lynn R C et al. Nature. 2019; 576(7786):293-300; incorporated herein by reference in its entirety.

The gene expression of several members of the AP-1 signalling pathway are altered in unactivated T cells following nucleofection compared with untreated cells and the consequences for T cell activity in vivo are unclear. In contrast, cells that are treated using SOLUPORE™ display a gene expression profile that is much closer to that of untreated cells indicated that these cells have been minimally perturbed and are likely to retain more normal activity in vivo.

The human amino acid sequence of AP-1 (SEQ ID NO: 27) is provided herein, and is publically available with GenBank Accession No: P05412.2, incorporated herein by reference.

| | |
|---|---|
| 1 | mtakmettfy ddalnasflp sesgpygysn pkilkqsmtl |
| | nladpvgslk phlraknsdl |
| 61 | ltspdvgllk laspelerli iqssnghitt tptptqflcp |
| | knvtdeqegf aegfvralae |
| 121 | lhsqntlpsv tsaaqpvnga gmvapavasv aggsgsggfs |
| | aslhseppvy anlsnfnpga |
| 181 | lssgggapsy gaaglafpaq pqqqqqpphh lpqqmpvqhp |
| | rlqalkeepq tvpempgetp |
| 241 | plspidmesq erikaerkrm rnriaaskcr krkleriarl |
| | eekvktlkaq nselastanm |
| 301 | lreqvaqlkq kvmnhvnsgc qlmltqqlqt f |

Exemplary landmark residues, domains, and fragments of AP-1 include, but are not limited to residues 255-310 (helical region), residues 255-306 (helical region), residues 8, 58, 63, 89, and 93 (phosphorylation). A fragment of an AP-1 protein is less than the length of the full length protein, e.g., a fragment is at least 3, 4, 5, 6, 7, 8, 9, 10, 20, 30, 40, 50, 100, 200 or more residues in length, but less than e.g., 331 residues in the case of AP-1 above.

Human AP-1 nucleic acid sequence (start and stop codons underlined) is provided below, and is available with GenBank Accession No: NM_005354.6, incorporated herein by reference (SEQ ID NO: 28).

| | |
|---|---|
| 1 | aggagccgcc gccagtggag ggccgggcgc tgcggccgcg |
| | gccggggcgg gcgcagggcc |
| 61 | gagcggacgg gggggcgcgg gccccccggg aggccgcggc |
| | cactcccccc cgggccggcg |
| 121 | cggcggggga ggcggaggat ggaaacaccc ttctacggcg |
| | atgaggcgct gagcggcctg |
| 181 | ggcggcggcg ccagtggcag cggcggcagc ttcgcgtccc |
| | cgggccgctt gttccccggg |
| 241 | gcgcccccga cggccgcggc cggcagcatg atgaagaagg |
| | acgcgctgac gctgagcctg |
| 301 | agtgagcagg tggcggcagc gctcaagcct gcggccgcgc |
| | cgcctcctac ccccctgcgc |
| 361 | gccgacggcg cccccagcgc ggcacccccc gacggcctgc |
| | tcgcctctcc cgacctgggg |
| 421 | ctgctgaagc tggcctcccc cgagctcgag cgcctcatca |
| | tccagtccaa cgggctggtc |
| 481 | accaccacgc cgacgagctc acagttcctc taccccaagg |
| | tggcggccag cgaggagcag |
| 541 | gagttcgccg agggcttcgt caaggccctg gaggatttac |
| | acaagcagaa ccagctcggc |
| 601 | gcgggcgcgg ccgctgccgc cgccgccgcc gccgccgggg |
| | ggccctcggg cacggccacg |
| 661 | ggctccgcgc cccccggcga gctggccccg gcggcggccg |
| | cgcccgaagc gcctgtctac |
| 721 | gcgaacctga gcagctacgc gggcggcgcc gggggcgcgg |
| | ggggcgccgc gacggtcgcc |
| 781 | ttcgctgccg aacctgtgcc cttcccgccg ccgccacccc |
| | caggcgcgtt ggggccgccg |
| 841 | cgcctggctg cgctcaagga cgagccacag acggtgcccg |
| | acgtgccgag cttcggcgag |
| 901 | agcccgccgt tgtcgcccat cgacatggac acgcaggagc |
| | gcatcaaggc ggagcgcaag |
| 961 | cggctgcgca accgcatcgc cgcctccaag tgccgcaagc |
| | gcaagctgga gcgcatctcg |
| 1021 | cgcctggaag agaaagtgaa gaccctcaag agtcagaaca |
| | cggagctggc gtccacggcg |
| 1081 | agcctgctgc gcgagcaggt ggcgcagctc aagcagaaag |
| | tcctcagcca cgtcaacagc |
| 1141 | ggctgccagc tgctgcccca gcaccaggtg cccgcgtact |
| | gagtccgcgc gcggggcgca |
| 1201 | tgcgcggcca ccctccccaa ggggcgggct cgcggggggg |
| | tgtcgtgggc gccccggact |
| 1261 | tggagagggt gcggccctgg ggaccccccc tccccgagtg |
| | tgcccaggaa ctcagagagg |
| 1321 | gcgcggcccc cggggattcc ccccccccga gggtgcccag |
| | gactcgacaa gctggacccc |
| 1381 | ctgctcccgg gggggcgagc gcatgacccc cccgccctcg |
| | cgctgcctct ttcccccgcg |
| 1441 | cggccgcccc gtgttgcaca aacccgcgcg tctcggctgc |
| | ccctttgtac accgcgccgc |
| 1501 | ggaagggggc tccgaggggg cgcagcctca aaccctgcct |
| | ttcctttact tttacttttt |
| 1561 | tttttttttc tttggaagag agaagaacag agtgttcgat |
| | tctgccctat ttatgtttct |
| 1621 | actcgggaac aaacgttggt tgtgtgtgtg tgtgttttct |
| | tgtgttggtt ttttaaagaa |
| 1681 | atgggaagaa gaaaaaaaaa ttctccgccc ctttcctcga |
| | tctcgctccc cccttcggtt |
| 1741 | ctttcgaccg gtcccccctc ccttttttgt tctgttttgt |
| | tttgttttgc tacgagtcca |
| 1801 | cattcctgtt tgtaatcctt ggttcgcccg gttttctgtt |
| | ttcagtaaag tctcgttacg |
| 1861 | ccagctcggc tctccgcctc cttcttcccc cgccggggcc |
| | tggcgggctg ggcggggcct |
| 1921 | ggttcgctt |

Exemplary landmark residues, domains, and fragments of AP-1 include, but are not limited to residues 139-1182 (coding region).

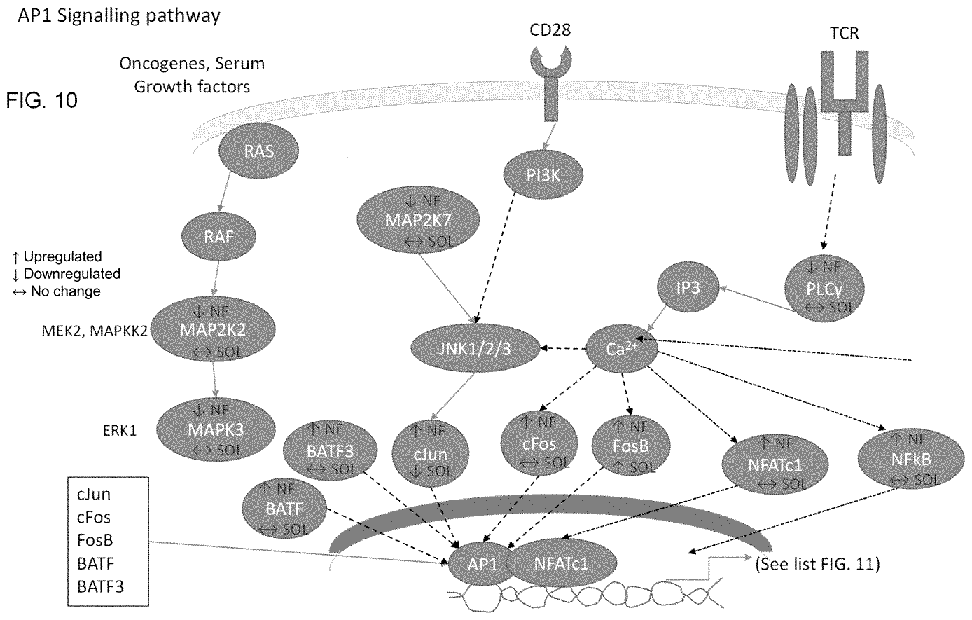

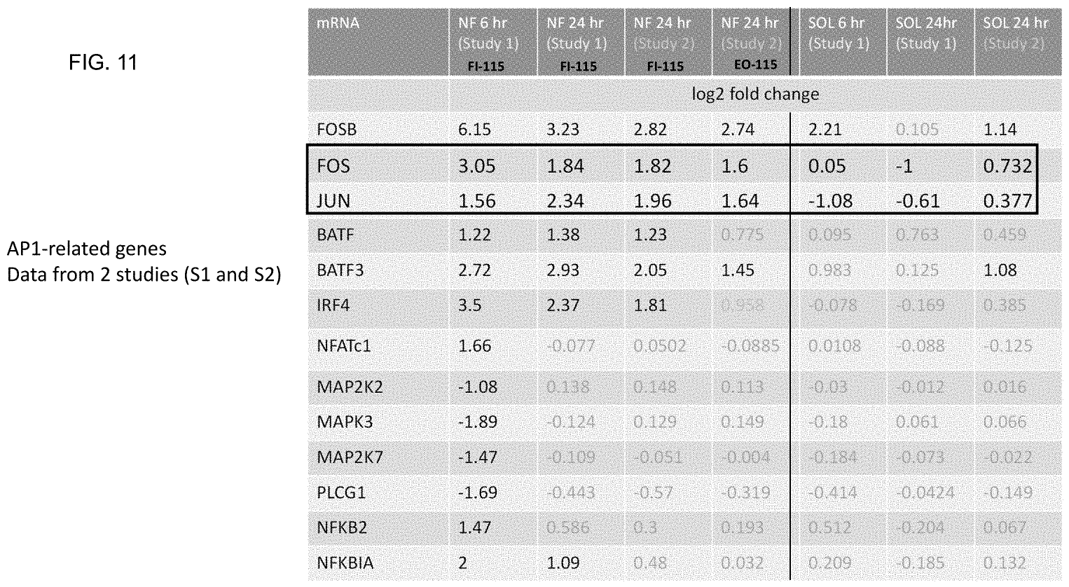

The AP-1 signaling pathway is depicted in FIG. 10. Relevant genes involved in the AP-1 signaling pathway include FOS, Jun, FOSB (Fos proto-oncogene), BATF (Basic leucine zipper transcriptional factor ATF-like), BATF3 (Basic leucine zipper transcriptional factor ATF-like 3), IRF4 (Interferon regulatory factor 4), NFATc1 (Nuclear factor of activated T-cells, cytoplasmic 1), MAP2K2 (dual specificity mitogen-activated protein kinase 2), MAPK3 (Mitogen-activated protein kinase 3), MAP2K7 (Dual specificity mitogen-activated protein kinase 7), PLCG1 (Phospholipase C, gamma 1), NFKB2 (Nuclear factor NF-kappa-B p100 subunit), NFKB1A (Nuclear factor NF-kappa-B p105 subunit).

The human amino acid sequence of Fosb (FBJ murine osteosarcoma viral oncogene homolog B) (SEQ ID NO: 3) is provided herein, and is publically available with GenBank Accession No: NP_001107643.1, incorporated herein by reference.

| | |
|---|---|
| 1 | mfqafpgdyd sgsrcsssps aesqylssvd sfgspptaaa |
| | sqecaglgem pgsfvptvta |
| 61 | ittsqdlqwl vqptlissma qsqgqplasq ppvvdpydmp |
| | gtsystpgms gyssggasgs |
| 121 | ggpstsgtts gpgparpara rprrpreete tdqleeekae |
| | leseiaelqk ekerlefvlv |
| 181 | ahkpgckipy eegpgpgpla evrdlpgsap akedgfswll |
| | ppppppplpf qtsqdappnl |
| 241 | taslfthsev qvlgdpfpvv npsytssfvl tcpevsafag |
| | aqrtsgsdqp sdplnspsll |
| 301 | al |

Exemplary landmark residues, domains, and fragments of Fosb include, but are not limited to residues 1-302 (coding region), residues 255-306 (helical region), residues 8, 58, 63, 89, and 93 (phosphorylation). A fragment of an FosB protein is less than the length of the full length protein, e.g., a fragment is at least 3, 4, 5, 6, 7, 8, 9, 10, 20, 30, 40, 50, 100, 200 or more residues in length, but less than e.g., 302 residues in the case of FosB above.

Human Fosb nucleic acid sequence (start and stop codons underlined) is provided below, and is available with GenBank Accession No: NM_006732.1, incorporated herein by reference (SEQ ID NO: 29).

| | |
|---|---|
| 1 | cattcataag actcagagct acggccacgg cagggacacg |
| | cggaaccaag acttggaaac |
| 61 | ttgattgttg tggttcttct tgggggttat gaaatttcat |
| | taatcttttt tttttccggg |
| 121 | gagaaagttt ttggaaagat tcttccagat atttcttcat |
| | tttcttttgg aggaccgact |
| 181 | tacttttttt ggtcttcttt attactcccc tccccccgtg |
| | ggacccgccg gacgcgtgga |
| 241 | ggagaccgta gctgaagctg attctgtaca gcgggacagc |
| | gctttctgcc cctgggggag |
| 301 | caacccctcc ctcgcccctg ggtcctacgg agcctgcact |
| | ttcaagaggt acagcggcat |
| 361 | cctgtggggg cctgggcacc gcaggaagac tgcacagaaa |
| | ctttgccatt gttggaacgg |
| 421 | gacgttgctc cttccccgag cttccccgga cagcgtactt |
| | tgaggactcg ctcagctcac |
| 481 | cggggactcc cacggctcac cccggacttg caccttactt |
| | ccccaacccg gccatagcct |
| 541 | tggcttcccg gcgacctcag cgtggtcaca ggggcccccc |
| | tgtgcccagg gaaatgtttc |
| 601 | aggctttccc cggagactac gactccggct cccggtgcag |
| | ctcctcaccc tctgccgagt |
| 661 | ctcaatatct gtcttcggtg gactccttcg gcagtccacc |
| | caccgccgcg gcctcccagg |
| 721 | agtgcgccgg tctcggggaa atgcccggtt ccttcgtgcc |
| | cacggtcacc gcgatcacaa |
| 781 | ccagccagga cctccagtgg cttgtgcaac ccaccctcat |
| | ctcttccatg gcccagtccc |
| 841 | aggggcagcc actggcctcc cagcccccgg tcgtcgaccc |
| | ctacgacatg ccgggaacca |
| 901 | gctactccac accaggcatg agtggctaca gcagtggcgg |
| | agcgagtggc agtggtgggc |
| 961 | cttccaccag cggaactacc agtgggcctg ggcctgcccg |
| | cccagcccga gcccggccta |
| 1021 | ggagaccccg agaggagacg ctcaccccag aggaagagga |
| | gaagcgaagg gtgcgccggg |
| 1081 | aacgaaataa actagcagca gctaaatgca ggaaccggcg |
| | gagggagctg accgaccgac |
| 1141 | tccaggcgga gacagatcag ttggaggaag aaaaagcaga |
| | gctggagtcg gagatcgccg |
| 1201 | agctccaaaa ggagaaggaa cgtctggagt ttgtgctggt |
| | ggcccacaaa ccgggctgca |
| 1261 | agatccccta cgaagagggg cccgggccgg gcccgctggc |
| | ggaggtgaga gatttgccgg |
| 1321 | gctcagcacc ggctaaggaa gatggcttca gctggctgct |
| | gccgcccccg ccaccaccgc |
| 1381 | ccctgccctt ccagaccagc caagacgcac cccccaacct |
| | gacggcttct ctctttacac |
| 1441 | acagtgaagt tcaagtcctc ggcgacccct tccccgttgt |
| | taacccttcg tacacttctt |
| 1501 | cgtttgtcct cacctgcccg gaggtctccg cgttcgccgg |
| | cgcccaacgc accagcggca |
| 1561 | gtgaccagcc ttccgatccc ctgaactcgc cctccctcct |
| | cgctcggtga actctttaga |
| 1621 | cacacaaaac aaacaaacac atgggggaga gagacttgga |
| | agaggaggag gaggaggaga |
| 1681 | aggaggagag agaggggaag agacaaagtg ggtgtgtggc |
| | ctccctggct cctccgtctg |
| 1741 | accctctgcg gccactgcgc cactgccatc ggacaggagg |
| | attccttgtg ttttgtcctg |
| 1801 | cctcttgttt ctgtgccccg gcgaggccgg agagctggtg |
| | actttgggga cagggggtgg |
| 1861 | gaaggggatg gacaccccca gctgactgtt ggctctctga |
| | cgtcaaccca agctctgggg |
| 1921 | atgggtgggg aggggggcgg gtgacgccca ccttcgggca |
| | gtcctgtgtg aggatgaagg |
| 1981 | gacgggggtg ggaggtaggc tgtggggtgg gctggagtcc |
| | tctccagaga ggctcaacaa |
| 2041 | ggaaaaatgc cactccctac ccaatgtctc ccacacccac |
| | cctttttttg gggtgcccag |
| 2101 | gttggtttcc cctgcactcc cgaccttagc ttattgatcc |
| | cacatttcca tggtgtgaga |
| 2161 | tcctctttac tctgggcaga agtgagcccc cccttaaagg |
| | gaattcgatg cccccctaga |
| 2221 | ataatctcat ccccccaccc gacttctttt gaaatgtgaa |
| | cgtccttcct tgactgtcta |
| 2281 | gccactccct cccagaaaaa ctggctctga ttggaatttc |
| | tggcctccta aggctcccca |
| 2341 | ccccgaaatc agcccccagc cttgtttctg atgacagtgt |
| | tatcccaaga ccctgccccc |
| 2401 | tgccagccga ccctcctggc cttcctcgtt gggccgctct |
| | gatttcaggc agcaggggct |
| 2461 | gctgtgatgc cgtcctgctg gagtgattta tactgtgaaa |
| | tgagttggcc agattgtggg |
| 2521 | gtgcagctgg gtggggcagc acacctctgg ggggataatg |
| | tccccactcc cgaaagcctt |
| 2581 | tcctcggtct cccttccgtc catccccctt cttcctcccc |
| | tcaacagtga gttagactca |
| 2641 | agggggtgac agaaccgaga agggggtgac agtcctccat |
| | ccacgtggcc tctctctctc |
| 2701 | tcctcaggac cctcagccct ggcctttttc tttaaggtcc |
| | cccgaccaat ccccagccta |
| 2761 | ggacgccaac ttctcccacc ccttggcccc tcacatcctc |
| | tccaggaagg cagtgagggg |
| 2821 | ctgtgacatt tttccggaga agatttcaga gctgaggctt |
| | tggtaccccc aaacccccaa |
| 2881 | tatttttgga ctggcagact caaggggctg gaatctcatg |
| | attccatgcc cgagtccgcc |
| 2941 | catccctgac catggttttg gctctcccac cccgccgttc |
| | cctgcgcttc atctcatgag |
| 3001 | gatttcttta tgaggcaaat ttatattttt taatatcggg |
| | gggtggacca cgccgccctc |
| 3061 | catccgtgct gcatgaaaaa cattccacgt gccccttgtc |
| | gcgcgtctcc catcctgatc |
| 3121 | ccagacccat tccttagcta tttatccctt tcctggtttc |
| | cgaaaggcaa ttatatctat |
| 3181 | tatgtataag taaatatatt atatatggat gtgtgtgtgt |
| | gcgtgcgcgt gagtgtgtga |
| 3241 | gcgcttctgc agcctcggcc taggtcacgt tggccctcaa |
| | agcgagccgt tgaattggaa |
| 3301 | actgcttcta gaaactctgg ctcagcctgt ctcgggctga |
| | cccttttctg atcgtctcgg |
| 3361 | cccctctgat tgttcccgat ggtctctctc cctctgtctt |
| | ttctcctccg cctgtgtcca |
| 3421 | tctgaccgtt ttcacttgtc tcctttctga ctgtccctgc |
| | caatgctcca gctgtcgtct |
| 3481 | gactctgggt tcgttgggga catgagattt tattttttgt |
| | gagtgagact gagggatcgt |
| 3541 | agatttttac aatctgtatc tttgacaatt ctgggtgcga |
| | gtgtgagagt gtgagcaggg |
| 3601 | cttgctcctg ccaaccacaa ttcaatgaat ccccgacccc |
| | cctaccccat gctgtacttg |
| 3661 | tggttctctt tttgtatttt gcatctgacc ccggggggct |
| | gggacagatt ggcaatgggc |
| 3721 | cgtcccctct ccccttggtt ctgcactgtt gccaataaaa |
| | agctcttaaa aacgc |

Exemplary landmark residues, domains, and fragments of Fosb include, but are not limited to residues 594-1610 (coding region), residues 3754-3759 (regulatory site), or residue 3775 (poly A site).

The human amino acid sequence of BATF (basic leucine zipper transcription factor, ATF-like (SEQ ID NO: 4) is provided herein, and is publically available with GenBank Accession No: CH471061.1, incorporated herein by reference.

| | |
|---|---|
| 1 | mphssdssds sfsrspppgk qdssddvrrv qrreknriaa |
| | qksrqrqtqk adtlhlesed |
| 61 | lekqnaalrk eikqlteelk yftsvlnshe plcsvlaast |
| | psppevvysa hafhqphvss |
| 121 | prfqp |

Exemplary landmark residues, domains, and fragments of BATF include, but are not limited to residues 1-125 (coding region). A fragment of an BATF protein is less than the length of the full length protein, e.g., a fragment is at least 3, 4, 5, 6, 7, 8, 9, 10, 20, 30, 40, 50, 100 or more residues in length, but less than e.g., 125 residues in the case of BATF above.

Human BATF nucleic acid sequence (start and stop codons underlined) is provided below, and is available with GenBank Accession No: NM_006399.3, incorporated herein by reference (SEQ ID NO: 30).

| | |
|---|---|
| 1 | caagagagag agagagcgtg caagccccaa agcgagcgac |
| | atgtcccttt ggggagcagt |
| 61 | ccctctgcac cccagagtga ggaggacgca ggggtcagag |
| | gtggctacag ggcaggcaga |
| 121 | ggaggcacct gtagggggtg gtgggctggt ggcccaggag |
| | aagtcaggaa gggagcccag |
| 181 | ctggtgacaa gagagcccag aggtgcctgg ggctgagtgt |
| | gagagcccgg aagatttcag |
| 241 | ccatgcctca cagctccgac agcagtgact ccagcttcag |
| | ccgctctcct ccccctggca |
| 301 | aacaggactc atctgatgat gtgagaagag ttcagaggag |
| | ggagaaaaat cgtattgccg |
| 361 | cccagaagag ccgacagagg cagacacaga aggccgacac |
| | cctgcacctg gagagcgaag |
| 421 | acctggagaa acagaacgcg gctctacgca aggagatcaa |
| | gcagctcaca gaggaactga |
| 481 | agtacttcac gtcggtgctg aacagccacg agcccctgtg |
| | ctcggtgctg gccgccagca |
| 541 | cgccctcgcc ccccgaggtg gtgtacagcg cccacgcatt |
| | ccaccaacct catgtcagct |
| 601 | ccccgcgctt ccagccctga gcttccgatg cggggagagc |
| | agagcctcgg gaggggcaca |
| 661 | cagactgtgg cagagctgcg cccatcccgc agaggcccct |
| | gtccacctgg agacccggag |
| 721 | acagaggcct ggacaaggag tgaacacggg aactgtcacg |
| | actggaaggg cgtgaggcct |
| 781 | cccagcagtg ccgcagcgtt tcgaggggcg tgtgctggac |
| | cccaccactg tgggttgcag |
| 841 | gcccaatgca gaagagtatt aagaaagatg ctcaagtccc |
| | atggcacaga gcaaggcggg |
| 901 | cagggaacgg ttatttttct aaataaatgc tttaaaagaa |
| | aaaaaaaaaa aaa |

Exemplary landmark residues, domains, and fragments of BATF include, but are not limited to residues 243-620 (coding region), residues 306-410 (exon), residues 411-941 (exon); residues 922-927 (polyA sequence); residue 941 (polyA site).

The human amino acid sequence of BATF3 (basic leucine zipper transcription factor, ATF-like 3) (SEQ ID NO: 5) is provided herein, and is publically available with GenBank Accession No: NP_061134.1, incorporated herein by reference.
1 msqglpaags vlqrsvaapg nqpqpqpqqq spedddrkvr rreknrvaaq rsrkkqtqka
61 dklheeyesl eqentmlrre igklteelkh ltealkehek mcplllcpmn fvpvpprpdp
121 vagclpr

Exemplary landmark residues, domains, and fragments of BATF3 include, but are not limited to residues 1-127 (coding region), residues 2 or 31 (phosphorylation site), residues 37-62 (basic motif), or residues 63-91 (leucine zipper). A fragment of an BATF3 protein is less than the length of the full length protein, e.g., a fragment is at least 3, 4, 5, 6, 7, 8, 9, 10, 20, 30, 40, 50, 100 or more residues in length, but less than e.g., 127 residues in the case of BATF3 above.

Human BATF3 nucleic acid sequence (start and stop codons underlined) is provided below, and is available with GenBank Accession No: NM_018664.2, incorporated herein by reference (SEQ ID NO: 31).

| | |
|---|---|
| 1 | ggggcagacg tgggacggga aggacggctg ccgggactgg |
| | cgcgcgggga cactgggccg |
| 61 | acgcgtggag tagcggggag agcgggaagc ctgagggggc |
| | ggggccggcg cgaggccgtg |
| 121 | ggtgcggcac gaggatgccg gcggcgggac agcgcccgta |
| | ggcagcccca cgggcagggc |
| 181 | gcgcgggcgg ggcggggcgg gccgggccag aggagcgccc |
| | ggcatgtcgc aagggctccc |
| 241 | ggccgccggc agcgtcctgc agaggagcgt cgcggcgccc |
| | gggaaccagc cgcagccgca |
| 301 | gccgcagcag cagagccctg aggatgatga caggaaggtc |
| | cgaaggagag aaaaaaaccg |
| 361 | agttgctgct cagagaagtc ggaagaagca gacccagaag |
| | gctgacaagc tccatgagga |
| 421 | atatgagagc ctggagcaag aaaacaccat gctgcggaga |
| | gagatcggga agctgacaga |
| 481 | ggagctgaag cacctgacag aggcactgaa ggagcacgag |
| | aagatgtgcc cgctgctgct |
| 541 | ctgccctatg aactttgtgc cagtgcctcc ccggccggac |
| | cctgtggccg gctgcttgcc |
| 601 | ccgatgaagc cggggacact cctctgccca gcaaggagcc |
| | ttggtcattt tcatacctgg |
| 661 | gaggaaggct tttccttcac aattgtatac agggggcacc |
| | tgtggccagg cctcctcctg |
| 721 | ggagctccag gaccagccag ctgtgttccc tgcagactgg |
| | gctcagcccg acatccaaca |
| 781 | ggcgccaaac tcacagagcc cttgtgcaga tccagcatgg |
| | aggccaccct caggagtgac |
| 841 | ttctcatcca ccctggcagc tagtaggttc tgctgttatg |
| | cagagccatt tcctctagaa |
| 901 | tttggataat aaagatgctt attgtctctc ccttctccag |
| | ttctgggaat ttacaggcac |
| 961 | aatacacttc cttttcctgg aaaaaaaaaa aa |

Exemplary landmark residues, domains, and fragments of BATF3 include, but are not limited to residues 224-607 (coding region), 314-418 (exon), 419-981 (exon), 908-913 (poly A signal sequence), or residue 926 or 981 (poly A site).

FOS (“c-Fos” or “v-Fos FBJ Murine Osteosarcoma Viral Oncogene Homolog, FBJ Murine Osteosarcoma Viral Oncogene Homolog”)

c-Fos is a proto-oncogene that is the human homolog of the retroviral oncogene v-fos. cFos is a part of a bigger Fos family of transcription factors which includes c-Fos, FosB, Fra-1 and Fra-2. c-Fos encodes a 62 kDa protein, which forms heterodimer with c-jun (part of Jun family of transcription factors), resulting in the formation of AP-1 (Activator Protein-1) complex which binds DNA at AP-1 specific sites at the promoter and enhancer regions of target genes and converts extracellular signals into changes of gene expression. It plays an important role in many cellular functions and has been found to be overexpressed in a variety of cancers.

The human amino acid sequence of FOS (SEQ ID NO: 32) is provided herein, and is publically available with GenBank Accession No: AY212879.1, incorporated herein by reference.

| | |
|---|---|
| 1 | mmfsgfnady easssrcssa spagdslsyy hspadsfssm |
| | gspvnaqdfc tdlavssanf |
| 61 | iptvtaists pdlqwlvqpa lvssvapsqt raphpfgvpa |
| | psagaysrag vvktmtggra |
| 121 | qsigrrgkve qlspeeeekr rirrernkma aakcrnrrre |
| | ltdtlqaetd qledeksalq |
| 181 | teianllkek eklefilaah rpackipddl gfpeemsvas |
| | ldltgglpev atpeseeaft |
| 241 | lpllndpepk psvepvksis smelktepfd dflfpassrp |
| | sgsetarsvp dmdlsgsfya |
| 301 | adweplhsgs lgmgpmatel eplctpvvtc tpsctaytss |
| | fvftypeads fpscaaahrk |
| 361 | gsssnepssd slssptllal |

Exemplary landmark residues, domains, and fragments of FOS include, but are not limited to residues 147-199 (coiled region). A fragment of an FOS protein is less than the length of the full length protein, e.g., a fragment is at least 3, 4, 5, 6, 7, 8, 9, 10, 20, 30, 40, 50, 100, 200 or more residues in length, but less than e.g., 380 residues in the case of FOS above.

Human FOS nucleic acid sequence (start and stop codons underlined) is provided below, and is available with GenBank Accession No: NM_005252.2, incorporated herein by reference (SEQ ID NO: 1).

| | |
|---|---|
| 1 | aaccgcatct gcagcgagca actgagaagc caagactgag |
| | ccggcggccg cggcgcagcg |
| 61 | aacgagcagt gaccgtgctc ctacccagct ctgcttcaca |
| | gcgcccacct gtctccgccc |
| 121 | ctcggcccct cgcccggctt tgcctaaccg ccacgatgat |
| | gttctcgggc ttcaacgcag |
| 181 | actacgaggc gtcatcctcc cgctgcagca gcgcgtcccc |
| | ggccggggat agcctctctt |
| 241 | actaccactc acccgcagac tccttctcca gcatgggctc |
| | gcctgtcaac gcgcaggact |
| 301 | tctgcacgga cctggccgtc tccagtgcca acttcattcc |
| | cacggtcact gccatctcga |
| 361 | ccagtccgga cctgcagtgg ctggtgcagc ccgccctcgt |
| | ctcctctgtg gccccatcgc |
| 421 | agaccagagc ccctcaccct ttcggagtcc ccgccccctc |
| | cgctggggct tactccaggg |
| 481 | ctggcgttgt gaagaccatg acaggaggcc gagcgcagag |
| | cattggcagg aggggcaagg |
| 541 | tggaacagtt atctccagaa gaagaagaga aaaggagaat |
| | ccgaagggaa aggaataaga |
| 601 | tggctgcagc caaatgccgc aaccggagga gggagctgac |
| | tgatacactc caagcggaga |
| 661 | cagaccaact agaagatgag aagtctgctt tgcagaccga |
| | gattgccaac ctgctgaagg |
| 721 | agaaggaaaa actagagttc atcctggcag ctcaccgacc |
| | tgcctgcaag atccctgatg |
| 781 | acctgggctt cccagaagag atgtctgtgg cttcccttga |
| | tctgactggg ggcctgccag |
| 841 | aggttgccac cccggagtct gaggaggcct tcaccctgcc |
| | tctcctcaat gaccctgagc |
| 901 | ccaagccctc agtggaacct gtcaagagca tcagcagcat |
| | ggagctgaag accgagccct |
| 961 | ttgatgactt cctgttccca gcatcatcca ggcccagtgg |
| | ctctgagaca gcccgctccg |
| 1021 | tgccagacat ggacctatct gggtccttct atgcagcaga |
| | ctgggagcct ctgcacagtg |
| 1081 | gctccctggg gatggggccc atggccacag agctggagcc |
| | cctgtgcact ccggtggtca |
| 1141 | cctgtactcc cagctgcact gcttacacgt cttccttcgt |
| | cttcacctac cccgaggctg |
| 1201 | actccttccc cagctgtgca gctgcccacc gcaagggcag |
| | cagcagcaat gagccttcct |
| 1261 | ctgactcgct cagctcaccc acgctgctgg ccctgtgagg |
| | gggcagggaa ggggaggcag |
| 1321 | ccggcaccca caagtgccac tgcccgagct ggtgcattac |
| | agagaggaga aacacatctt |
| 1381 | ccctagaggg ttcctgtaga cctagggagg accttatctg |
| | tgcgtgaaac acaccaggct |
| 1441 | gtgggcctca aggacttgaa agcatccatg tgtggactca |
| | agtccttacc tcttccggag |
| 1501 | atgtagcaaa acgcatggag tgtgtattgt tcccagtgac |
| | acttcagaga gctggtagtt |
| 1561 | agtagcatgt tgagccaggc ctgggtctgt gtctcttttc |
| | tctttctcct tagtcttctc |
| 1621 | atagcattaa ctaatctatt gggttcatta ttggaattaa |
| | cctggtgctg gatattttca |
| 1681 | aattgtatct agtgcagctg attttaacaa taactactgt |
| | gttcctggca atagtgtgtt |
| 1741 | ctgattagaa atgaccaata ttatactaag aaaagatacg |
| | actttatttt ctggtagata |
| 1801 | gaaataaata gctatatcca tgtactgtag tttttcttca |
| | acatcaatgt tcattgtaat |
| 1861 | gttactgatc atgcattgtt gaggtggtct gaatgttctg |
| | acattaacag ttttccatga |
| 1921 | aaacgtttta ttgtgttttt aatttattta ttaagatgga |
| | ttctcagata tttatatttt |
| 1981 | tattttattt ttttctacct tgaggtcttt tgacatgtgg |
| | aaagtgaatt tgaatgaaaa |
| 2041 | atttaagcat tgtttgctta ttgttccaag acattgtcaa |
| | taaa |

Exemplary landmark residues, domains, and fragments of FOS include, but are not limited to residues 156-1298 (coding region), 1803-1808 (polyA region), and 2079-2084 (polyA region).

Jun (“v-Jun Avian Sarcoma Virus 17 Oncogene Homolog, Jun Oncogene” or “c-Jun”)

c-Jun is a protein that in humans is encoded by the JUN gene. c-Jun, in combination with c-Fos, forms the AP-1 early response transcription factor. c-jun was the first oncogenic transcription factor discovered. The proto-oncogene c-Jun is the cellular homolog of the viral oncoprotein v-jun (P05411). The human JUN encodes a protein that is highly similar to the viral protein, which interacts directly with specific target DNA sequences to regulate gene expression.

Both Jun and its dimerization partners in AP-1 formation are subject to regulation by diverse extracellular stimuli, which include peptide growth factors, pro-inflammatory cytokines, oxidative and other forms of cellular stress, and UV irradiation. For example, UV irradiation is a potent inducer for elevated c-jun expression. c-jun transcription is autoregulated by its own product, Jun. The binding of Jun (AP-1) to a high-affinity AP-1 binding site in the jun promoter region induces jun transcription. This positive autoregulation by stimulating its own transcription may be a mechanism for prolonging the signals from extracellular stimuli. This mechanism can have biological significance for the activity of c-jun in cancer.

Phosphorylation of Jun at serines 63 and 73 and threonine 91 and 93 increases transcription of the c-jun target genes. Therefore, regulation of c-jun activity can be achieved through N-terminal phosphorylation by the Jun N-terminal kinases (JNKs). It is shown that Jun's activity (AP-1 activity) in stress-induced apoptosis and cellular proliferation is regulated by its N-terminal phosphorylation.

The human amino acid sequence of Jun (SEQ ID NO: 33) is provided herein, and is publically available with GenBank Accession No: AAV38564.1, incorporated herein by reference.

| | |
|---|---|
| 1 | mtakmettfy ddalnasflp sesgpygysn pkilkqsmtl |
| | nladpvgslk phlraknsdl |
| 61 | ltspdvgllk laspelerli iqssnghitt tptptqflcp |
| | knvtdeqegf aegfvralae |
| 121 | lhsqntlpsv tsaaqpvnga gmvapavasv aggsgsggfs |
| | aslhseppvy anlsnfnpga |
| 181 | lssgggapsy gaaglafpaq pqqqqqpphh lpqqmpvqhp |
| | rlqalkeepq tvpempgetp |
| 241 | plspidmesq erikaerkrm rnriaaskcr krkleriarl |
| | eekvktlkaq nselastanm |
| 301 | lreqvaqlkq kvmnhvnsgc qlmltqqlqt f |

Exemplary landmark residues, domains, and fragments of Jun include, but are not limited to residues 255-306 (coiled coil region). A fragment of an Jun protein is less than the length of the full length protein, e.g., a fragment is at least 3, 4, 5, 6, 7, 8, 9, 10, 20, 30, 40, 50, 100, 200 or more residues in length, but less than e.g., 331 residues in the case of Jun above.

Human Jun nucleic acid sequence (start and stop codons underlined) is provided below, and is available with GenBank Accession No: NM_002228.3, incorporated herein by reference (SEQ ID NO: 2).

| | |
|---|---|
| 1 | gacatcatgg gctattttta ggggttgact ggtagcagat |
| | aagtgttgag ctcgggctgg |
| 61 | ataagggctc agagttgcac tgagtgtggc tgaagcagcg |
| | aggcgggagt ggaggtgcgc |
| 121 | ggagtcaggc agacagacag acacagccag ccagccaggt |
| | cggcagtata gtccgaactg |
| 181 | caaatcttat tttcttttca ccttctctct aactgcccag |
| | agctagcgcc tgtggctccc |
| 241 | gggctggtgt ttcgggagtg tccagagagc ctggtctcca |
| | gccgcccccg ggaggagagc |
| 301 | cctgctgccc aggcgctgtt gacagcggcg gaaagcagcg |
| | gtacccacgc gcccgccggg |
| 361 | ggaagtcggc gagcggctgc agcagcaaag aactttcccg |
| | gctgggagga ccggagacaa |
| 421 | gtggcagagt cccggagcga acttttgcaa gcctttcctg |
| | cgtcttaggc ttctccacgg |
| 481 | cggtaaagac cagaaggcgg cggagagcca cgcaagagaa |
| | gaaggacgtg cgctcagctt |
| 541 | cgctcgcacc ggttgttgaa cttgggcgag cgcgagccgc |
| | ggctgccggg cgccccctcc |
| 601 | ccctagcagc ggaggagggg acaagtcgtc ggagtccggg |
| | cggccaagac ccgccgccgg |
| 661 | ccggccactg cagggtccgc actgatccgc tccgcgggga |
| | gagccgctgc tctgggaagt |
| 721 | gagttcgcct gcggactccg aggaaccgct gcgcccgaag |
| | agcgctcagt gagtgaccgc |
| 781 | gacttttcaa agccgggtag cgcgcgcgag tcgacaagta |
| | agagtgcggg aggcatctta |
| 841 | attaaccctg cgctccctgg agcgagctgg tgaggagggc |
| | gcagcgggga cgacagccag |
| 901 | cgggtgcgtg cgctcttaga gaaactttcc ctgtcaaagg |
| | ctccgggggg cgcgggtgtc |
| 961 | ccccgcttgc cagagccctg ttgcggcccc gaaacttgtg |
| | cgcgcagccc aaactaacct |
| 1021 | cacgtgaagt gacggactgt tctatgactg caaagatgga |
| | aacgaccttc tatgacgatg |
| 1081 | ccctcaacgc ctcgttcctc ccgtccgaga gcggacctta |
| | tggctacagt aaccccaaga |
| 1141 | tcctgaaaca gagcatgacc ctgaacctgg ccgacccagt |
| | ggggagcctg aagccgcacc |
| 1201 | tccgcgccaa gaactcggac ctcctcacct cgcccgacgt |
| | ggggctgctc aagctggcgt |
| 1261 | cgcccgagct ggagcgcctg ataatccagt ccagcaacgg |
| | gcacatcacc accacgccga |
| 1321 | cccccaccca gttcctgtgc cccaagaacg tgacagatga |
| | gcaggagggc ttcgccgagg |
| 1381 | gcttcgtgcg cgccctggcc gaactgcaca gccagaacac |
| | gctgcccagc gtcacgtcgg |
| 1441 | cggcgcagcc ggtcaacggg gcaggcatgg tggctcccgc |
| | ggtagcctcg gtggcagggg |
| 1501 | gcagcggcag cggcggcttc agcgccagcc tgcacagcga |
| | gccgccggtc tacgcaaacc |
| 1561 | tcagcaactt caacccaggc gcgctgagca gcggcggcgg |
| | ggcgccctcc tacggcgcgg |
| 1621 | ccggcctggc ctttcccgcg caaccccagc agcagcagca |
| | gccgccgcac cacctgcccc |
| 1681 | agcagatgcc cgtgcagcac ccgcggctgc aggccctgaa |
| | ggaggagcct cagacagtgc |
| 1741 | ccgagatgcc cggcgagaca ccgcccctgt cccccatcga |
| | catggagtcc caggagcgga |
| 1801 | tcaaggcgga gaggaagcgc atgaggaacc gcatcgctgc |
| | ctccaagtgc cgaaaaagga |
| 1861 | agctggagag aatcgcccgg ctggaggaaa aagtgaaaac |
| | cttgaaagct cagaactcgg |
| 1921 | agctggcgtc cacggccaac atgctcaggg aacaggtggc |
| | acagcttaaa cagaaagtca |
| 1981 | tgaaccacgt taacagtggg tgccaactca tgctaacgca |
| | gcagttgcaa acattttgaa |
| 2041 | gagagaccgt cgggggctga ggggcaacga agaaaaaaaa |
| | taacacagag agacagactt |
| 2101 | gagaacttga caagttgcga cggagagaaa aaagaagtgt |
| | ccgagaacta aagccaaggg |
| 2161 | tatccaagtt ggactgggtt gcgtcctgac ggcgccccca |
| | gtgtgcacga gtgggaagga |
| 2221 | cttggcgcgc cctcccttgg cgtggagcca gggagcggcc |
| | gcctgcgggc tgccccgctt |
| 2281 | tgcggacggg ctgtccccgc gcgaacggaa cgttggactt |
| | ttcgttaaca ttgaccaaga |
| 2341 | actgcatgga cctaacattc gatctcattc agtattaaag |
| | gggggagggg gagggggtta |
| 2401 | caaactgcaa tagagactgt agattgcttc tgtagtactc |
| | cttaagaaca caaagcgggg |
| 2461 | ggagggttgg ggaggggcgg caggagggag gtttgtgaga |
| | gcgaggctga gcctacagat |
| 2521 | gaactctttc tggcctgcct tcgttaactg tgtatgtaca |
| | tatatatatt ttttaatttg |
| 2581 | atgaaagctg attactgtca ataaacagct tcatgccttt |
| | gtaagttatt tcttgtttgt |
| 2641 | ttgtttgggt atcctgccca gtgttgtttg taaataagag |
| | atttggagca ctctgagttt |
| 2701 | accatttgta ataaagtata taattttttt atgttttgtt |
| | tctgaaaatt ccagaaagga |
| 2761 | tatttaagaa aatacaataa actattggaa agtactcccc |
| | taacctcttt tctgcatcat |
| 2821 | ctgtagatac tagctatcta ggtggagttg aaagagttaa |
| | gaatgtcgat taaaatcact |
| 2881 | ctcagtgctt cttactatta agcagtaaaa actgttctct |
| | attagacttt agaaataaat |
| 2941 | gtacctgatg tacctgatgc tatggtcagg ttatactcct |
| | cctcccccag ctatctatat |
| 3001 | ggaattgctt accaaaggat agtgcgatgt ttcaggaggc |
| | tggaggaagg ggggttgcag |
| 3061 | tggagaggga cagcccactg agaagtcaaa catttcaaag |
| | tttggattgt atcaagtggc |
| 3121 | atgtgctgtg accatttata atgttagtag aaattttaca |
| | ataggtgctt attctcaaag |
| 3181 | caggaattgg tggcagattt tacaaaagat gtatccttcc |
| | aatttggaat cttctctttg |
| 3241 | acaattccta gataaaaaga tggcctttgc ttatgaatat |
| | ttataacagc attcttgtca |
| 3301 | caataaatgt attcaaatac caaaaaaaaa aaaaaaaa |

Exemplary landmark residues, domains, and fragments of Jun include, but are not limited to residues 1044-2039 (coding region), 3302-3307 (regulatory region), 2624 (polyA region).

### T Cell Exhaustion

T-cell “exhaustion” is referred to the state of T cells that respond poorly because of prolonged antigen exposure during chronic viral infections or cancer. “T cell exhaustion” is characterized by loss of T cell function, which may occur as a result of an infection or a disease. Exhausted T cells display a transcriptional program distinct from that of functional effector or memory T cells, characterized by the expression of inhibitory cell surface receptors including PD-1 (programmed death protein 1), LAG-3 (Lymphocyte-activation gene 3), TIM-3 (T-cell immunoglobulin mucin-3), TIGIT (T cell immunoreceptor with Ig and ITIM domains), and CTLA-4 (cytotoxic T-lymphocyte-associated protein 4), and by impaired IL-2 (interleukin 2), TNF (tumor necrosis factor), and IFN-γ (interferon gamma) cytokine production. NFAT (Nuclear factor of activated T-cells) and AP-1 transcription factors synergistically play a central role in inducing hyporesponsive states, such as anergy and exhaustion. Exhausted cells exhibit low expression of AP-1 factors (FOS, FOSB, and Jun). See, for example, Wherry, J. and Kurachi, M. “Molecular and cellular insights into T cell exhaustion” Nat Rev Immunol. 2015 August; 15(8): 486-499, incorporated herein by reference in its entirety.

### Effects of the SOLUPORE™ Process T Cell Functionality

The data described herein provide an understanding of the SOLUPORE™ process on T cell functionality. Specifically, the functionality of T cells was compared with cells transfected by nucleofection and electroporation. In examples, soluporation, nucleofection and electroporation (both of which utilize the application of an electrical current to cells), including with no cargo (e.g., mock), or model cargo (e.g., mRNA-GFP) was compared and evaluated. With the above-described transfection methods, a number of functionality assays were performed, including 1) phenotypic analysis, 2) cytokine release, 3) gene expression profiling of approximately greater than 700 immune related genes, and 4) metabolic rate.

### Cytokine Release Upon Immune Cell Transfection

Viral delivery systems to engineer cells are susceptible to vector-mediated genotoxicity, leading to adverse immunogenicity and severe side effects. Electroporation is a commonly used tool to delivery exogenous material into cells for therapeutic purposes, but a consequence of electroporation-induced disruptions includes non-specific release of cytokines. Using the SOLUPORE™ delivery method described herein, no significant difference was seen with the SOLUPORE™ delivery method compared to untreated control cells. In contrast, significant differences are observed in electroporated immune cells, such as T cells.

For example, the cytokines that were not perturbed using the methods described herein include IL-2 (interleukin 2), IFN-γ (interferon gamma), TNFα(tumor necrosis factor alpha), GM-CSF (Granulocyte-macrophage colony-stimulating factor), IL-8 (interleukin 8), IL-10 (interleukin 10), MIP-1α(macrophage inflammatory protein 1 alpha), MIP-1β (macrophage inflammatory protein 1 beta), Fractalkine, ITAC (Interferon—inducible T Cell Alpha Chemoattractant) and IL-17A (interleukin 17A). In contrast, electroporated cells exhibited significant differences in IL-2 and IL-8.

The human amino acid sequence of IL-2 (SEQ ID NO: 34) is provided herein, and is publically available with GenBank Accession No: NP_000577.2, incorporated herein by reference.

| | |
|---|---|
| 1 | myrmqllsci alslalvtns aptssstkkt qlqlehllld |
| | lqmilnginn yknpkltrml |
| 61 | tfkfympkka telkhlqcle eelkpleevl nlaqsknfhl |
| | rprdlisnin vivlelkgse |
| 121 | ttfmceyade tativeflnr witfcgsiis tlt |

Exemplary landmark residues, domains, and fragments of IL-2 include, but are not limited to a fragment less than the length of the full length protein, e.g., a fragment is at least 3, 4, 5, 6, 7, 8, 9, 10, 20, 30, 40, 50, 100, or more residues in length, but less than e.g., 153 residues in the case of IL-2 above.

Human IL-2 nucleic acid sequence (start and stop codons underlined) is provided below, and is available with GenBank Accession No: NM_000586.2, incorporated herein by reference (SEQ ID NO: 17).

| | |
|---|---|
| 1 | cgaattcccc tatcacctaa gtgtgggcta atgtaacaaa |
| | gagggatttc acctacatcc |
| 61 | attcagtcag tctttggggg tttaaagaaa ttccaaagag |
| | tcatcagaag aggaaaaatg |
| 121 | aaggtaatgt tttttcagac aggtaaagtc tttgaaaata |
| | tgtgtaatat gtaaaacatt |
| 181 | ttgacacccc cataatattt ttccagaatt aacagtataa |
| | attgcatctc ttgttcaaga |
| 241 | gttccctatc actctcttta atcactactc acagtaacct |
| | caactcctgc cacaatgtac |
| 301 | aggatgcaac tcctgtcttg cattgcacta agtcttgcac |
| | ttgtcacaaa cagtgcacct |
| 361 | acttcaagtt ctacaaagaa aacacagcta caactggagc |
| | atttactgct ggatttacag |
| 421 | atgattttga atggaattaa taattacaag aatcccaaac |
| | tcaccaggat gctcacattt |
| 481 | aagttttaca tgcccaagaa ggccacagaa ctgaaacatc |
| | ttcagtgtct agaagaagaa |
| 541 | ctcaaacctc tggaggaagt gctaaattta gctcaaagca |
| | aaaactttca cttaagaccc |
| 601 | agggacttaa tcagcaatat caacgtaata gttctggaac |
| | taaagggatc tgaaacaaca |
| 661 | ttcatgtgtg aatatgctga tgagacagca accattgtag |
| | aatttctgaa cagatggatt |
| 721 | accttttgtc aaagcatcat ctcaacactg acttgataat |
| | taagtgcttc ccacttaaaa |
| 781 | catatcaggc cttctattta tttaaatatt taaattttat |
| | atttattgtt gaatgtatgg |
| 841 | tttgctacct attgtaacta ttattcttaa tcttaaaact |
| | ataaatatgg atcttttatg |
| 901 | attctttttg taagccctag gggctctaaa atggtttcac |
| | ttatttatcc caaaatattt |
| 961 | attattatgt tgaatgttaa atatagtatc tatgtagatt |
| | ggttagtaaa actatttaat |
| 1021 | aaatttgata aatataaaaa aaaaaaa |

Exemplary landmark residues, domains, and fragments of IL-2 include, but are not limited to residues 295-756 (coding region), 295-354 (signal peptide), 355-753 (mature peptide).

The human amino acid sequence of IL-8 (SEQ ID NO: 35) is provided herein, and is publically available with GenBank Accession No: NP_001341769.1, incorporated herein by reference.

| | |
|---|---|
| 1 | mtsklavall aaflisaalc egavlprsak elrcqcikty |
| | skpfhpkfik elrviesgph |
| 61 | canteiivkl sdgrelcldp kenwvqrvve kflkr |

Exemplary landmark residues, domains, and fragments of IL-8 include, but are not limited to a fragment less than the length of the full length protein, e.g., a fragment is at least 3, 4, 5, 6, 7, 8, 9, 10, 20, 30, 40, 50, or more residues in length, but less than e.g., 95 residues in the case of IL-8 above. Exemplary landmark residues, domains, and fragments of IL-8 include, but are not limited to residues 1-95 (protein precursor); residues 1-20 (signal peptide).

Human IL-8 nucleic acid sequence (start and stop codons underlined) is provided below, and is available with GenBank Accession No: NM_001354840.3, incorporated herein by reference (SEQ ID NO: 18).

| | |
|---|---|
| 1 | acaaactttc agagacagca gagcacacaa gcttctagga |
| | caagagccag gaagaaacca |
| 61 | ccggaaggaa ccatctcact gtgtgtaaac atgacttcca |
| | agctggccgt ggctctcttg |
| 121 | gcagccttcc tgatttctgc agctctgtgt gaaggtgcag |
| | ttttgccaag gagtgctaaa |
| 181 | gaacttagat gtcagtgcat aaagacatac tccaaacctt |
| | tccaccccaa atttatcaaa |
| 241 | gaactgagag tgattgagag tggaccacac tgcgccaaca |
| | cagaaattat tgtaaagctt |
| 301 | tctgatggaa gagagctctg tctggacccc aaggaaaact |
| | gggtgcagag ggttgtggag |
| 361 | aagtttttga agaggtaagt tatatatttt ttaatttaaa |
| | tttttcattt atcctgagac |
| 421 | atataatcca aagtcagcct ataaatttct ttctgttgct |
| | aaaaatcgtc attaggtatc |
| 481 | tgcctttttg gttaaaaaaa aaaggaatag catcaatagt |
| | gagtttgttg tactcatgac |
| 541 | cagaaagacc atacatagtt tgcccaggaa attctgggtt |
| | taagcttgtg tcctatactc |
| 601 | ttagtaaagt tctttgtcac tcccagtagt gtcctatttt |
| | agatgataat ttctttgatc |
| 661 | tccctattta tagttgagaa tatagagcat ttctaacaca |
| | tgaatgtcaa agactatatt |
| 721 | gacttttcaa gaaccctact ttccttctta ttaaacatag |
| | ctcatcttta tatttttaat |
| 781 | tttattttag ggctgagaat tcataaaaaa attcattctc |
| | tgtggtatcc aagaatcagt |
| 841 | gaagatgcca gtgaaacttc aagcaaatct acttcaacac |
| | ttcatgtatt gtgtgggtct |
| 901 | gttgtagggt tgccagatgc aatacaagat tcctggttaa |
| | atttgaattt cagtaaacaa |
| 961 | tgaatagttt ttcattgtac catgaaatat ccagaacata |
| | cttatatgta aagtattatt |
| 1021 | tatttgaatc tacaaaaaac aacaaataat ttttaaatat |
| | aaggattttc ctagatattg |
| 1081 | cacgggagaa tatacaaata gcaaaattga ggccaagggc |
| | caagagaata tccgaacttt |
| 1141 | aatttcagga attgaatggg tttgctagaa tgtgatattt |
| | gaagcatcac ataaaaatga |
| 1201 | tgggacaata aattttgcca taaagtcaaa tttagctgga |
| | aatcctggat ttttttctgt |
| 1261 | taaatctggc aaccctagtc tgctagccag gatccacaag |
| | tccttgttcc actgtgcctt |
| 1321 | ggtttctcct ttatttctaa gtggaaaaag tattagccac |
| | catcttacct cacagtgatg |
| 1381 | ttgtgaggac atgtggaagc actttaagtt ttttcatcat |
| | aacataaatt attttcaagt |
| 1441 | gtaacttatt aacctattta ttatttatgt atttatttaa |
| | gcatcaaata tttgtgcaag |
| 1501 | aatttggaaa aatagaagat gaatcattga ttgaatagtt |
| | ataaagatgt tatagtaaat |
| 1561 | ttattttatt ttagatatta aatgatgttt tattagataa |
| | atttcaatca gggtttttag |
| 1621 | attaaacaaa caaacaattg ggtacccagt taaattttca |
| | tttcagataa acaacaaata |
| 1681 | attttttagt ataagtacat tattgtttat ctgaaatttt |
| | aattgaacta acaatcctag |
| 1741 | tttgatactc ccagtcttgt cattgccagc tgtgttggta |
| | gtgctgtgtt gaattacgga |
| 1801 | ataatgagtt agaactatta aaacagccaa aactccacag |
| | tcaatattag taatttcttg |
| 1861 | ctggttgaaa cttgtttatt atgtacaaat agattcttat |
| | aatattattt aaatgactgc |
| 1921 | atttttaaat acaaggcttt atatttttaa ctttaagatg |
| | tttttatgtg ctctccaaat |
| 1981 | tttttttact gtttctgatt gtatggaaat ataaaagtaa |
| | atatgaaaca tttaaaatat |
| 2041 | aatttgttgt caaagtaa |

Exemplary landmark residues, domains, and fragments of IL-8 include, but are not limited to residues 91-378 (coding region) or 91-150 (signal peptide).

### CAR Plus Delivery

CAR plus refers to a population of cells that have been either 1) virally transduced, and then followed by additional intracellular delivery method (e.g., the SOLUPORE™ delivery method, electroporation, or nucleofection, or any combination thereof), or 2) the SOLUPORE™ delivery method was used to deliver exogenous cargo, and then the cells are subjected to an additional intracellular delivery method (e.g., viral transduction, the SOLUPORE™ delivery method, electroporation, or nucleofection, or any combination thereof). Where cells have first been virally transduced, and then subjected to intracellular delivery using the SOLUPORE™ delivery method, viral components may still be present.

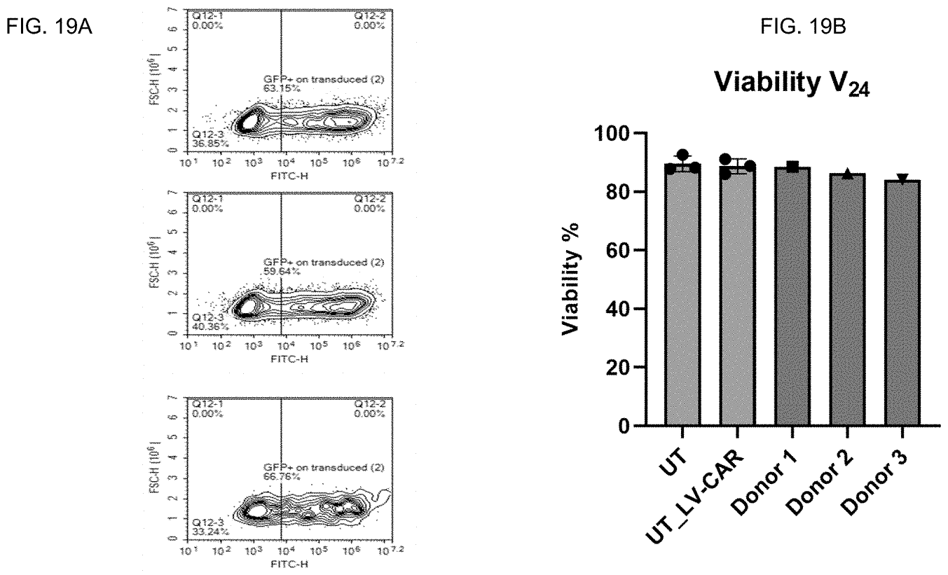

The SOLUPORE™ delivery method was used in conjunction with cells that have undergone an additional cargo delivery manipulation method. For example, the SOLUPORE™ delivery method was used to delivery exogenous cargo, e.g., mRNA, to cells that had already been virally transduced (FIG. 19A and FIG. 19B). Alternatively, the SOLUPORE™ delivery method is used first to deliver exogenous cargo, e.g., mRNA, and then the cells are subjected to an additional delivery manipulation method, e.g., viral transduction.

Exemplary additional intracellular delivery methods include the SOLUPORE™ delivery method, viral transduction, electroporation, nucleofection, or any combination thereof. Exemplary viruses that may be used for intracellular delivery include a lentivirus, a retrovirus, an adenovirus, an adeno-associated virus (AAV), or a herpes simplex virus (HSV). In preferred examples, the virus is a lentivirus.

### Summary of Viruses Used for Gene Delivery Applications

| Virus | Description | Advantages | Disadvantages |
|---|---|---|---|
| Adenoviruses | non-enveloped | efficient in a | high |
| (AdVs) | dsDNA-virus able | broad range of | immunogenicity; |
| | to carry ≤8 kbp | host cells | transient |
| | DNA | | expression |
| Adeno- | non-enveloped | efficient in a | small carrying |
| associated | recombinant | broad range of | capacity |
| viruses | ssDNA-virus with | host cells; non- | |
| (AAVs) | a small carrying | inflammatory/ | |
| | capacity (≤4 kbp) | pathogenic | |
| Retroviruses | enveloped ssRNA- | long-term | limited tropism |
| | carrying virus | expression | to dividing cells; |
| | with ≤8 kbp | | random integration |
| | RNA capacity | | |
| Lentiviruses | enveloped ssRNA- | efficient in a | potential oncogenic |
| | carrying virus | broad range of | responses |
| | with ≤8 kbp | host cells; long- | |
| | RNA capacity | term expression | |
| Herpes simplex | enveloped dsDNA- | efficient in a | potential |
| viruses (HSV)-1 | virus with >30 | broad range of | inflammatory |
| large packing | kbp carrying | host cells | responses; |
| capacity; | capacity | | transient |
| | | | expression |

Pharmaceutics 2020, 12, 183, incorporated herein by reference in its entirety

### Gene Editing and Indel (Insertion Deletion) Analysis

Gene editing is a type of genetic engineering in which DNA is inserted, deleted, modified or replaced in site specific locations in the genome of a cell. Common methods for such editing use engineered nucleases that create site-specific double-strand breaks (DSBs) at desired locations in the genome. The induced double-strand breaks are repaired through nonhomologous end-joining (NHEJ) or homology-directed repair (HDR), resulting in targeted mutations (“edits”). NHEJ can lead to gene disruption through the introduction of insertions, deletions, translocations, or other DNA rearrangements at the site of a DSB. Alternatively, a precise DNA edit can be made by supplying a donor DNA template encoding the desired DNA change flanked by sequence homologous to the region upstream and downstream of the DSB. Cellular homology-directed repair (HDR) then results in the incorporation of sequence from the exogenous DNA template at the DSB site.

Electroporation can cause cell damage and stress which in turn leads to reduced cell proliferation rates. The effects of electroporation may make the DNA repair pathways that are necessary for gene editing less efficient, resulting in lower efficiencies of gene edit. The SOLUPORE™ delivery method does not damage cells or reduce cell proliferation, and thus it is more suitable than electroporation for achieving efficient levels of gene editing.

In addition, in order to generate effector cells that are suitable for allogeneic applications or for targeting solid tumors, it will be necessary carry out complex editing. However, if multiple nucleases and DNA templates are used simultaneously in cells, multiple DSBs will occur and it will not be possible to control where in the genome each templates will be inserted. Therefore, it is desirable to carry out multiple edits in sequence rather than simultaneously in order to ensure that a given exogenous DNA template inserts into the desired region. However, because electroporation technologies are harsh and cause cell damage, it is very challenging to carry out multiple rounds of electroporation. In contrast, the SOLUPORE™ technology is gentle on cells and it can carry out multiple sequential transfections (see Example 2). This enables greater control over complex editing regimes in cells.

### Exogenous Cargo

The exogenous cargo (or “payload”) delivered to the immune cell describes a compound, or composition that is delivered via an aqueous solution across a cell plasma membrane and into the interior of a cell. The exogenous cargo can include a nucleic acid (for example, RNA (ribonucleic acid), mRNA (messenger RNA), or DNA (deoxyribonucleic acid)), a protein or peptide, a small chemical molecule, or any combination thereof. The small chemical molecule can be less than 1,000 Da. A small molecule is a compound that is less than 2000 Daltons in mass. The molecular mass of the small molecule is preferably less than 1000 Daltons, more preferably less than 600 Daltons, e.g., the compound is less than 500 Daltons, 400 Daltons, 300 Daltons, 200 Daltons, or 100 Daltons.

In preferred examples, the exogenous cargo comprises nucleic acid, e.g., messenger RNA (mRNA). The exogenous cargo comprising mRNA include CD19 CAR—2^{nd} Generation mRNA (SEQ ID NO: 6), CD19 CAR—3^{rd} Generation mRNA (SEQ ID NO: 8), TRAIL-DR5 (TNF-related apoptosis-inducing ligand (TRAIL) Death Receptor 5) variant mRNA (SEQ ID NO: 10), TRAIL (SEQ ID NO: 11), IL-15 (interleukin 15) mRNA, TCR (T cell receptor) mRNA.

In other examples, the exogenous cargo comprises Cas9 (CRISPR associated protein 9) protein, for example with guide RNAs including TRAC (T cell receptor alpha constant SEQ ID NO: 25) or PD-1 (programmed death ligand 1 SEQ ID NO: 26). In other examples, the exogenous cargo comprises Cas12a protein (CRISPR associated protein 12a) including guide RNAs including TRAC and PD-1. In examples, the exogenous cargo comprises MAD7 protein (see, Price M A, et al, Rosser S J. Expanding and understanding the CRISPR toolbox for Bacillus subtilis with MAD7 and dM D7 Biotechnol Bioeng. 2020; 117 (6): 1805-1816, incorporated herein by reference), with guide RNAs including TRAC or PD-1. In examples, the exogenous cargo comprises SgCas (see, Petri s G, et al. Hit and go CAS9 delivered through a lentiviral based self-limiting circuit. Nat Commun. 2017; 8: 15334. Published 2017 May 22, incorporated herein by reference), with guide RNAs including TRAC or PD-1. In examples, the exogenous cargo comprises Cas13, with guide RNAs including TRAC or PD-1. Alternatively, the exogenous cargo comprises base editors such as Cas9n, or zinc finger nucleases, or MegaTALs.

In examples, the exogenous cargo comprises the Sleeping Beauty 100 transposon/transposase system, or the Sleeping Beauty 1000 transposon/transposase system, or the Piggy Bac transposon/transposase system, or the TcBuster transposon/transposase system.

In other examples, the exogenous cargo comprises DNA, for example, CD19 CAR DNA, TRAIL DNA, or IL-15 DNA.

In examples, the exogenous cargo comprises the Yamanaka factors used for generation of stable induced pluripotent stem cells from adult human cells. For example, the Yamanaka factors comprise c-Myc (MYC proto-oncogene, bHLH transcription factor), Klf4 (Kruppel Like Factor 4), Oct4 (octamer-binding transcription factor 4), or Sox2 (SRY (sex determining region Y)-box 2).

In further examples, the exogenous cargo comprises siRNA (small interfering RNA), for example against PD-1. In further examples, the exogenous cargo comprises shRNA (small hairpin RNA), for example shRNA against PD-1.

The CD19 CAR mRNA sequence is provided below (SEQ ID NO: 6)
ATGGCTCTCCCAGTGACTGCCCTACTGCTTCCCCTAGCGCTGCTGCTGCATGCGGCGCGCCCGGACATCCAGATGAC
CCAGACCACCTCCAGCCTGAGCGCCAGCCTGGGCGACCGGGTGACCATCAGCTGCCGGGCCAGCCAGGACATCAGCA
AGTACCTGAACTGGTATCAGCAGAAGCCCGACGGCACCGTCAAGCTGCTGATCTACCACACCAGCCGGCTGCACAGC
GGCGTGCCCAGCCGGTTTAGCGGCAGCGGCTCCGGCACCGACTACAGCCTGACCATCTCCAACCTGGAACAGGAAGA
TATCGCCACCTACTTTTGCCAGCAGGGCAACACACTGCCCTACACCTTTGGCGGCGGAACAAAGCTGGAAATCACCG
GCGGAGGCGGATCTGGCGGCGGAGGATCTGGGGGAGGCGGCTCTGAGGTGAAGCTGCAGGAAAGCGGCCCTGGCCTG
GTGGCCCCCAGCCAGAGCCTGAGCGTGACCTGCACCGTGAGCGGCGTGAGCCTGCCCGACTACGGCGTGAGCTGGAT
CCGGCAGCCCCCCAGGAAGGGCCTGGAATGGCTGGGCGTGATCTGGGGCAGCGAGACCACCTACTACAACAGCGCCC
TGAAGAGCCGGCTGACCATCATCAAGGACAACAGCAAGAGCCAGGTGTTCCTGAAGATGAACAGCCTGCAGACCGAC
GACACCGCCATCTACTACTGCGCCAAGCACTACTACTACGGCGGCAGCTACGCCATGGACTACTGGGGCCAGGGCAC
CAGCGTGACCGTGAGC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

Signal (underlined)
Vl-cd19 (bold)
(G45)3 LINKER (italics)
VH-cd19 (Bold + Underlined)
cd28-Hinge/TM/cO-STIMULATORY DOMAIN ( [symbol] [symbol] )
cd3Z-SIGNALLING DOMAIN ( [symbol] [symbol] [symbol] )
The CD19 CAR protein sequence is provided below (SEQ ID NO: 7)
MALPVTALLLPLALLLHAARPDIQMTQTTSSLSASLGDRVTISCRASQDISKYLNWYQQKPDGTVKLLIYHTSRLHS
GVPSRFSGSGSGTDYSLTISNLEQEDIATYFCQQGNTLPYTFGGGTKLEITGGGGSGGGGSGGGGSEVKLQESGPGL
VAPSQSLSVTCTVSGVSLPDYGVSWIRQPPRKGLEWLGVIWGSETTYYNSALKSRLTIIKDNSKSQVFLKMNSLQTD
DTAIYYCAKHYYYGGSYAMDYWGQGTSVTVSAAAIEVMYPPPYLDNEKSNGTIIHVKGKHLCPSPLFPGPSKPFWVL
VVVGGVLACYSLLVTVAFIIFWVRSKRSRLLHSDYMNMTPRRPGPTRKHYQPYAPPRDFAAYRSRVKFSRSADAPAY
QQGQNQLYNELNLGRREEYDVLDKRRGRDPEMGGKPRRKNPQEGLYNELQKDKMAEAYSEIGMKGERRRGKGHDGLY
QGLSTATKDTYDALHMQALPPR
Avectas CD19 CAR mRNA sequence is provided below (SEQ ID NO: 8)
TGATATCCAGATGACCCAGACCACCAGCAGCCTGTCTGCCTCTCTGGGCGATAGAGTGACCATCAGCTGTAGAGCCA
GCCAGGACATCAGCAAGTACCTGAACTGGTATCAGCAGAAACCCGACGGCACCGTGAAGCTGCTGATCTACCACACC
AGCAGACTGCACAGCGGCGTGCCAAGCAGATTTTCTGGCAGCGGCTCTGGCACCGACTACAGCCTGACAATCAGCAA
CCTGGAACAAGAGGATATCGCTACCTACTTCTGCCAGCAAGGCAACACCCTGCCTTACACCTTTGGCGGAGGCACCA
AGCTGGAAATCACCGGCTCTACAAGCGGCAGCGGCAAACCTGGATCTGGCGAGGGATCTACCAAGGGCGAAGTGAAA
CTGCAAGAGTCTGGCCCTGGACTGGTGGCCCCATCTCAGTCTCTGAGCGTGACCTGTACAGTCAGCGGAGTGTCCCT
GCCTGATTACGGCGTGTCCTGGATCAGACAGCCTCCTCGGAAAGGCCTGGAATGGCTGGGAGTGATCTGGGGCAGCG
AGACAACCTACTACAACAGCGCCCTGAAGTCCCGGCTGACCATCATCAAGGACAACTCCAAGAGCCAGGTGTTCCTG
AAGATGAACAGCCTGCAGACCGACGACACCGCCATCTACTATTGCGCCAAGCACTACTACTACGGCGGCAGCTACGC
CATGGATTATTGGGGCCAGGGCACCAGCGTGACCGTGTCTAGTACAACAACCCCTGCTCCTCGGCCTCCTACACCAG
CTCCTACAATTGCCAGCCAGCCACTGTCTCTGAGGCCCGAAGCTTGTAGACCTGCTGCTGGCGGAGCCGTGCATACA
AGAGGACTGGATTTCGCCTGCGACTTCTGGGTGCTCGTGGTTGTTGGCGGAGTGCTGGCCTGTTACAGCCTGCTGGT
TACCGTGGCCTTCATCATCTTTTGGGTCCGAAGCAAGCGGAGCCGGCTGCTGCACTCCGACTACATGAACATGACCC
CTAGACGGCCCGGACCTACCAGAAAGCACTACCAGCCTTACGCTCCTCCTAGAGACTTCGCCGCCTACAGATCCAAG
CGGGGCAGAAAGAAACTGCTCTACATCTTCAAGCAGCCCTTCATGCGGCCCGTGCAGACCACACAAGAGGAAGATGG
CTGCTCCTGCAGATTCCCCGAGGAAGAAGAAGGCGGCTGCGAGCTGAGAGTGAAGTTCAGCAGATCCGCCGACGCTC
CCGCCTATAAGCAGGGACAGAACCAGCTGTACAACGAGCTGAACCTGGGGAGAAGAGAAGAGTACGACGTGCTGGAC
AAGCGGAGAGGCAGGGATCCTGAAATGGGCGGCAAGCCCAGACGGAAGAATCCTCAAGAGGGCCTGTATAATGAGCT
GCAGAAAGACAAGATGGCCGAGGCCTACAGCGAGATCGGAATGAAGGGCGAGCGCAGAAGAGGCAAGGGACACGATG
GACTGTACCAGGGACTGAGCACCGCCACCAAGGATACCTATGACGCCCTGCACATGCAGGCCCTGCCTCCAAGATAA
GTCGACAATCAA
Avectas CD19 CAR protein sequence is provided below (SEQ ID NO: 9)
DIQMTQTTSSLSASLGDRVTISCRASQDISKYLNWYQQKPDGTVKLLIYHTSRLHSGVPSRFSGSGSGTDYSLTISN
LEQEDIATYFCQQGNTLPYTFGGGTKLEITGSTSGSGKPGSGEGSTKGEVKLQESGPGLVAPSQSLSVTCTVSGVSL
PDYGVSWIRQPPRKGLEWLGVIWGSETTYYNSALKSRLTIIKDNSKSQVFLKMNSLQTDDTAIYYCAKHYYYGGSYA
MDYWGQGTSVTVSSTTTPAPRPPTPAPTIASQPLSLRPEACRPAAGGAVHTRGLDFACDFWVLVVVGGVLACYSLLV
TVAFIIFWVRSKRSRLLHSDYMNMTPRRPGPTRKHYQPYAPPRDFAAYRSKRGRKKLLYIFKQPFMRPVQTTQEEDG
CSCRFPEEEEGGCELRVKFSRSADAPAYKQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
Full length E195R/D269H TRAIL (DR5)-variant: (846 nucleotides) mRNA is
provided below (SEQ ID NO: 10)
ATG GCT ATG ATG GAG GTC CAG GGG GGA CCC AGC CTG GGA CAG ACC TGC GTG CTG ATC
GTG ATC TTC ACA GTG CTC CTG CAG TCT CTC TGT GTG GCT GTA ACT TAC GTG TAC TTT
ACC AAC GAG CTG AAG CAG ATG CAG GAC AAG TAC TCC AAA AGT GGC ATT GCT TGT TTC
TTA AAA GAA GAT GAC AGT TAT TGG GAC CCC AAT GAC GAA GAG AGT ATG AAC AGC CCC
TGC TGG CAA GTC AAG TGG CAA CTC CGT CAG CTC GTT AGA AAG ATG ATT TTG AGA ACC
TCT GAG GAA ACC ATT TCT ACA GTT CAA GAA AAG CAA CAA AAT ATT TCT CCC CTA
GTG AGA GAA AGA GGT CCT CAG AGA GTA GCA GCT CAC ATA ACT GGG ACC AGA GGA AGA
AGC AAC ACA TTG TCT TCT CCA AAC TCC AAG AAT GAA AAG GCT CTG GGC CGC AAA ATA
AAC TCC TGG GAA TCA TCA AGG AGT GGG CAT TCA TTC CTG AGC AAC TTG CAC TTG AGG
AAT GGT GAA CTG GTC ATC CAT GAA AAA GGG TTT TAC TAC ATC TAT TCC CAA ACA TAC
TTT CGA TTT CAG GAG CGA ATA AAA GAA AAC ACA AAG AAC GAC AAA CAA ATG GTC CAA
TAT ATT TAC AAA TAC ACA AGT TAT CCT GAC CCT ATA TTG TTG ATG AAA AGT GCT AGA
AAT AGT TGT TGG TCT AAA GAT GCA GAA TAT GGA CTC TAT TCC ATC TAT CAA GGG GGA
ATA TTT GAG CTT AAG GAA AAT GAC AGA ATT TTT GTT TCT GTA ACA AAT GAG CAC TTG
ATA GAC ATG CAC CAT GAA GCC AGT TTT TTC GGG GCC TTT TTA GTT GGC TAA
Full Length E195R/D269H TRAIL (DR5 Variant) protein sequence is provided
below (SEQ ID NO: 36) Note E195R/D269H are bold and underlined.
MAMMEVQGGPSLGQTCVLIVIFTVLLQSLCVAVTYVYFTNELKQMQDKYSKSGIACFLKE
DDSYWDPNDEESMNSPCWQVKWQLRQLVRKMILRTSEETISTVQEKQQNISPLVRERGPQ
RVAAHITGTRGRSNTLSSPNSKNEKALGRKINSWESSRSGHSFLSNLHLRNGELVIHEKG
FYYIYSQTYFRFQERIKENTKNDKQMVQYIYKYTSYPDPILLMKSARNSCWSKDAEYGLY
SIYQGGIFELKENDRIFVSVTNEHLIDMHHEASFFGAFLVG
Full Length TRAIL protein sequence is provided below (SEQ ID NO: 11)
MAMMEVQGGPSLGQTCVLIVIFTVLLQSLCVAVTYVYFTNELKQMQDKYSKSGIACFLKE
DDSYWDPNDEESMNSPCWQVKWQLRQLVRKMILRTSEETISTVQEKQQNISPLVRERGPQ
RVAAHITGTRGRSNTLSSPNSKNEKALGRKINSWESSRSGHSFLSNLHLRNGELVIHEKG
FYYIYSQTYFRFQEEIKENTKNDKQMVQYIYKYTSYPDPILLMKSARNSCWSKDAEYGLY
SIYQGGIFELKENDRIFVSVTNEHLIDMDHEASFFGAFLVG
RNPs
The sequence for human TRAC targeting gRNA was AGAGTCTCTCAGCTGGTACA (SEQ ID
NO: 25) and for human PDCD1 targeting gRNA was GTCTGGGCGGTGCTACAACT (SEQ ID
NO: 26).
Human cDNAs for Oct4, Sox2, Klf4, and c-Myc were amplified by RT-PCR from human
ES poly(A+)RNA using the following primers: 5′-GGA TCC GAA TTC ATG GCG GGA CAC
CTG GCT TCGG-3′ (SEQ ID NO: 15) and 5′-AAA AAA GTC GAC GCG GCG TCT GCG
TCT GCG GCG TCT GCG GTT TGA ATG CAT GGG AGA GCC-3′ (SEQ ID NO: 38) for
human Oct4, 5′-GGA TCC GAA TTC ATG TAC AAC ATG ATG GAG ACG G-3′ (SEQ ID
NO: 16) and 5′-AAA AAA CTC GAG GCG GCG TCT GCG TCT GCG GCG TCT GCG CAT
GTG CGA CAG GGG CAG TG-3′ (SEQ ID NO: 39) for human Sox2, 5′-GGA TCC GAA TTC
ATG GCT GTC AGC GAC GCG CTG C-3′ (SEQ ID NO: 14) and 5′-AAA AAA CTC GAG
GCG GCG TCT GCG TCT GCG GCG TCT GCG AAA GTG CCT CTT CAT GTG TAA GGC-3′
(SEQ ID NO: 37) for human Klf4, and 5′-GGA TCC GAA TTC ATG CCC CTC AAC GTT
AGC TTC AC3' (SEQ ID NO: 13) and 5'-AAA AAA CTC GAG GCG GCG TCT GCG TCT
GCG GCG TCT GCG CGC ACA AGA GTT CCG TAG CTG TTC-3′ (SEQ ID NO: 36) for
human c-Myc.
Streptococcus pyogenes Cas9 NCBI Reference Sequence: NZ_CP010450.1
(SEQ ID NO: 19), incorporated herein by reference
MDKKYSIGLDIGTNSVGWAVITDDYKVPSKKFKVLGNTDRHSIKKNLIGALLFDSGETAEATRLKRTARRRYTRRKN
RICYLQEIFSNEMAKVDDSFFHRLEESFLVEEDKKHERHPIFGNIVDEVAYHEKYPTIYHLRKKLADSTDKADLRLI
YLALAHMIKFRGHFLIEGDLNPDNSDVDKLFIQLVQTYNQLFEENPINASRVDAKAILSARLSKSRRLENLIAQLPG
EKKNGLFGNLIALLLGLTPNFKSNFDLAEDAKLQLSKDTYDDDLDNLLAQIGDQYADLFLAAKNLSDAILLSDILRV
NSEITKAPLSASMIKRYDEHHQDLTLLKALVRQQLPEKYKEIFFDQSKNGYAGYIDGGASQEEFYKFIKPILEKMDG
TEELLAKLNREDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFNASLGTYHDLLKIIKDKDFLDNEENEDIL
EDIVLTLTLFEDKEMIEERLKKYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHKPENIVIEMARENQTT
QKGQKNSRERMKRIEEGIKELGSDILKEYPVENTQLQNEKLYLYYLQNGRDMYVDQELDINRLSDYDVDHIVPQSFL
KDDSIDNKVLTRSDKNRGKSDNVPSEEVVKKMKNYWKQLLNAKLITQRKFDNLTKAERGGLSELDKAGFIKRQLVET
RQITKHVAQILDSRMNTKYDENDKLIREVRVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLIETNGETGEIVWDKGR
DFATVRKVLSMPQVNIVKKTEVQTGGFSKESILPKRNSDKLIARKKDWDPKKYGGFDSPTVAYSVLVVAKVEKGKSK
KLKSVKELLGITIMERSSFEKNPIDFLEAKGYKEVRKDLIIKLPKYSLFELENGRKRMLASAGELQKGNELALPSKY
VNFLYLASHYEKLKGSPEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
Staphylococcus agnetis Cas9 NCBI Reference Sequence: NZ_CP045927.1
(SEQ ID NO: 20), incorporated herein by reference.
MNNYILGLDIGITSVGYGIVDSDTREIKDAGVRLFPEANVDNNEGRRSKRGARRLKRRRIHRLDRVKHLLAEYNLLD
LTNIPKSTNPYQIRVKGLNEKLSKDELVIALLHIAKRRGIHNVNVMMDDNDSGNELSTKDQLKKNAKALSDKYVCEL
QLERFEQDYKVRGEKNRFKTEDFVREARKLLETQSKFFEIDQTFIMRYIDLVETRREYFEGPGKGSPFGWEGNIKKW
FEQMMGHCTYFPEELRSVKYAYSAELFNALNDLNNLVITRDEEAKLNYGEKFQIIENVFKQKKTPNLKQIAKEIGVS
ETDIKGYRVNKSGKPEFTQFKLYHDLKNIFEDSKYLNDVQLMDNIAEIITIYQDPESIIKELNQLPELLSEKEKEKI
SALSGYAGTHRLSLKCINLLLDDLWESSLNQMELFTKLNLKPKKIDLSQQHKIPIKLVDDFILSPVVKRAFIQSIQV
VNAIIDKYGLPEDIIIELARENNSDDRRKFLNQLQKQNAETRKQVEKVLREYGNDNAKRIVQKIKLHNMQEGKCLYS
LKDIPLEDLLKNPNHYEVDHIIPRSVAFDNSMHNKVLVRAEENSKKGNRTPYQYLNSSESSLSYNEFKQHILNLSKT
KDRITKKKREYLLEERDINKYDVQKEFINRNLVDTRYATRELTSLLKAYFSANNLDVKVKTINGSFTNYLRKVWKFD
KDRNKGYKHHAEDALIIANADFLFKHNKKLRNINKVLDAPSKEVDKKRVTVQSEDEYNQMFEDTQKAQAIKKFEIRK
FSHRVDKKPNRQLIKDTLYSTRNIDGIEYVVESIKDIYSVNNDKVKTKFKKDPHRLLMYRNDPQTFEKFEKVFKQYE
SEKNPFAKYYEETGEKIRKFSKTGQGPYINKIKYLRERLGRHCDVTNKYINSRNKIVQLKIYSYRFDIYQYGNNYKM
ITISYIDLEQKSNYYYISREKYEQKKKDKQIDDSYKFIGSFYKNDIINYNGEMYRVIGVNDSEKIKFSLI
Synthetic construct derived from Staphylococcus aureus Cas9 NCBI Reference
Sequence: MN548085.1 (SEQ ID NO: 21); incorporated herein by reference.
MAPKKKRKVGIHGVPAAKRNYILGLDIGITSVGYGIIDYETRDVIDAGVRLFKEANVENNEGRRSKRGARRLKRRRR
HRIQRVKKLLFDYNLLTDHSELSGINPYEARVKGLSQKLSEEEFSAALLHLAKRRGVHNVNEVEEDTGNELSTREQI
SRNSKALEEKYVAELQLERLKKDGEVRGSINRFKTSDYVKEAKQLLKVQKAYHQLDQSFIDTYIDLLETRRTYYEGP
GEGSPFGWKDIKEWYEMLMGHCTYFPEELRSVKYAYNADLYNALNDLNNLVITRDENEKLEYYEKFQIIENVFKQKK
KPTLKQIAKEILVNEEDIKGYRVTSTGKPEFTNLKVYHDIKDITARKEIIENAELLDQIAKILTIYQSSEDIQEELT
NLNSELTQEEIEQISNLKGYTGTHNLSLKAINLILDELWHTNDNQIAIFNRLKLVPKKVDLSQQKEIPTTLVDDFIL
SPVVKRSFIQSIKVINAIIKKYGLPNDIIIELAREKNSKDAQKMINEMQKRNRQTNERIEEIIRTTGKENAKYLIEK
IKLHDMQEGKCLYSLEAIPLEDLLNNPFNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTRYATRGLMNLLRSYFRVNNLDVKVKSIN
GGFTSFLRRKWKFKKERNKGYKHHAEDALIIANADFIFKEWKKLDKAKKVMENQMFEERQAESMPEIETEQEYKEIF
ITPHQIKHIKDFKDYKYSHRVDKKPNRELINDTLYSTRKDDKGNTLIVNNLNGLYDKDNDKLKKLINKSPEKLLMYH
HDPQTYQKLKLIMEQYGDEKNPLYKYYEETGNYLTKYSKKDNGPVIKKIKYYGNKLNAHLDITDDYPNSRNKVVKLS
LKPYRFDVYLDNGVYKFVTVKNLDVIKKENYYEVNSKCYEEAKKLKKISNQAEFIASFYNNDLIKINGELYRVIGVN
NDLLNRIEVNMIDITYREYLENMNDKRPPRIIKTIASKTQSIKKYSTDILGNLYEVKSKKHPQIIKKGKRPAATKKA
GQAKKKKGSYPYDVPDYASGFANELGPRLMGK
Candidatus Methanomethylophilus alvus Mx1201 Cas12a NCBI Reference Sequence:
NC_020913.1 (SEQ ID NO: 22), incorporated herein by reference.
MHTGGLLSMDAKEFTGQYPLSKTLRFELRPIGRTWDNLEASGYLAEDRHRAECYPRAKELLDDNHRAFLNRVLPQID
MDWHPIAEAFCKVHKNPGNKELAQDYNLQLSKRRKEISAYLQDADGYKGLFAKPALDEAMKIAKENGNESDIEVLEA
FNGFSVYFTGYHESRENIYSDEDMVSVAYRITEDNFPRFVSNALIFDKLNESHPDIISEVSGNLGVDDIGKYFDVSN
YNNFLSQAGIDDYNHIIGGHTTEDGLIQAFNVVLNLRHQKDPGFEKIQFKQLYKQILSVRTSKSYIPKQFDNSKEMV
DCICDYVSKIEKSETVERALKLVRNISSFDLRGIFVNKKNLRILSNKLIGDWDAIETALMHSSSSENDKKSVYDSAE
AFTLDDIFSSVKKFSDASAEDIGNRAEDICRVISETAPFINDLRAVDLDSLNDDGYEAAVSKIRESLEPYMDLFHEL
EIFSVGDEFPKCAAFYSELEEVSEQLIEIIPLFNKARSFCTRKRYSTDKIKVNLKFPTLADGWDLNKERDNKAAILR
KDGKYYLAILDMKKDLSSIRTSDEDESSFEKMEYKLLPSPVKMLPKIFVKSKAAKEKYGLTDRMLECYDKGMHKSGS
AFDLGFCHELIDYYKRCIAEYPGWDVFDFKFRETSDYGSMKEFNEDVAGAGYYMSLRKIPCSEVYRLLDEKSIYLFQ
IYNKDYSENAHGNKNMHTMYWEGLFSPQNLESPVFKLSGGAELFFRKSSIPNDAKTVHPKGSVLVPRNDVNGRRIPD
SIYRELTRYFNRGDCRISDEAKSYLDKVKTKKADHDIVKDRRFTVDKMMFHVPIAMNFKAISKPNLNKKVIDGIIDD
QDLKIIGIDRGERNLIYVTMVDRKGNILYQDSLNILNGYDYRKALDVREYDNKEARRNWTKVEGIRKMKEGYLSLAV
SKLADMIIENNAIIVMEDLNHGFKAGRSKIEKQVYQKFESMLINKLGYMVLKDKSIDQSGGALHGYQLANHVTTLAS
VGKQCGVIFYIPAAFTSKIDPTTGFADLFALSNVKNVASMREFFSKMKSVIYDKAEGKFAFTFDYLDYNVKSECGRT
LWTVYTVGERFTYSRVNREYVRKVPTDIIYDALQKAGISVEGDLRDRIAESDGDTLKSIFYAFKYALDMRVENREED
YIQSPVKNASGEFFCSKNAGKSLPQDSDANGAYNIALKGILQLRMLSEQYDPNAESIRLPLITNKAWLTFMQSGMKT
WKN
Candidatus Methanomethylophilus alvus isolate MGYG-HGUT-02456 Cas12a NCBI
Reference Sequence: NZ_LR699000.1 (SEQ ID NO: 23), incorporated herein by reference.
MDAKEFTGQYPLSKTLRFELRPIGRTWDNLEASGYLAEDRHRAECYPRAKELLDDNHRAFLNRVLPQIDMDWHPIAE
AFCKVHKNPGNKELAQDYNLQLSKRRKEISAYLQDADGYKGLFAKPALDEAMKIAKENGNESDIEVLEAFNGFSVYF
TGYHESRENIYSDEDMVSVAYRITEDNFPRFVSNALIFDKLNESHPDIISEVSGNLGVDDIGKYFDVSNYNNFLSQA
GIDDYNHIIGGHTTEDGLIQAFNVVLNLRHQKDPGFEKIQFKQLYKQILSVRTSKSYIPKQFDNSKEMVDCICDYVS
KIEKSETVERALKLVRNISSFDLRGIFVNKKNLRILSNKLIGDWDAIETALMHSSSSENDKKSVYDSAEAFTLDDIF
SSVKKFSDASAEDIGNRAEDICRVISETAPFINDLRAVDLDSLNDDGYEAAVSKIRESLEPYMDLFHELEIFSVGDE
FPKCAAFYSELEEVSEQLIEIIPLFNKARSFCTRKRYSTDKIKVNLKFPTLADGWDLNKERDNKAAILRKDGKYYLA
ILDMKKDLSSIRTSDEDESSFEKMEYKLLPSPVKMLPKIFVKSKAAKEKYGLTDRMLECYDKGMHKSGSAFDLGFCH
ELIDYYKRCIAEYPGWDVFDFKFRETSDYGSMKEFNEDVAGAGYYMSLRKIPCSEVYRLLDEKSIYLFQIYNKDYSE
NAHGNKNMHTMYWEGLFSPQNLESPVFKLSGGAELFFRKSSIPNDAKTVHPKGSVLVPRNDVNGRRIPDSIYRELTR
YFNRGDCRISDEAKSYLDKVKTKKADHDIVKDRRFTVDKMMFHVPIAMNFKAISKPNLNKKVIDGIIDDQDLKIIGI
DRGERNLIYVTMVDRKGNILYQDSLNILNGYDYRKALDVREYDNKEARRNWTKVEGIRKMKEGYLSLAVSKLADMII
ENNAIIVMEDLNHGFKAGRSKIEKQVYQKFESMLINKLGYMVLKDKSIDQSGGALHGYQLANHVTTLASVGKQCGVI
FYIPAAFTSKIDPTTGFADLFALSNVKNVASMREFFSKMKSVIYDKAEGKFAFTFDYLDYNVKSECGRTLWTVYTVG
ERFTYSRVNREYVRKVPTDIIYDALQKAGISVEGDLRDRIAESDGDTLKSIFYAFKYALDMRVENREEDYIQSPVKN
ASGEFFCSKNAGKSLPQDSDANGAYNIALKGILQLRMLSEQYDPNAESIRLPLITNKAWLTFMQSGMKTWKN
Candidatus Methanoplasma termitum strain MpT1 chromosome Cas12a NCBI
Reference Sequence: NZ_CP010070.1 (SEQ ID NO: 24), incorporated by reference.
MNNYDEFTKLYPIQKTIRFELKPQGRTMEHLETFNFFEEDRDRAEKYKILKEAIDEYHKKFIDEHLTNMSLDWNSLK
QISEKYYKSREEKDKKVFLSEQKRMRQEIVSEFKKDDRFKDLFSKKLFSELLKEEIYKKGNHQEIDALKSFDKFSGY
FIGLHENRKNMYSDGDEITAISNRIVNENFPKFLDNLQKYQEARKKYPEWIIKAESALVAHNIKMDEVFSLEYFNKV
LNQEGIQRYNLALGGYVTKSGEKMMGLNDALNLAHQSEKSSKGRIHMTPLFKQILSEKESFSYIPDVFTEDSQLLPS
IGGFFAQIENDKDGNIFDRALELISSYAEYDTERIYIRQADINRVSNVIFGEWGTLGGLMREYKADSINDINLERTC
KKVDKWLDSKEFALSDVLEAIKRTGNNDAFNEYISKMRTAREKIDAARKEMKFISEKISGDEESIHIIKTLLDSVQQ
FLHFFNLFKARQDIPLDGAFYAEFDEVHSKLFAIVPLYNKVRNYLTKNNLNTKKIKLNFKNPTLANGWDQNKVYDYA
SLIFLRDGNYYLGIINPKRKKNIKFEQGSGNGPFYRKMVYKQIPGPNKNLPRVFLTSTKGKKEYKPSKEIIEGYEAD
KHIRGDKFDLDFCHKLIDFFKESIEKHKDWSKFNFYFSPTESYGDISEFYLDVEKQGYRMHFENISAETIDEYVEKG
DLFLFQIYNKDFVKAATGKKDMHTIYWNAAFSPENLQDVVVKLNGEAELFYRDKSDIKEIVHREGEILVNRTYNGRT
PVPDKIHKKLTDYHNGRTKDLGEAKEYLDKVRYFKAHYDITKDRRYLNDKIYFHVPLTLNFKANGKKNLNKMVIEKF
LSDEKAHIIGIDRGERNLLYYSIIDRSGKIIDQQSLNVIDGFDYREKLNQREIEMKDARQSWNAIGKIKDLKEGYLS
KAVHEITKMAIQYNAIVVMEELNYGFKRGRFKVEKQIYQKFENMLIDKMNYLVFKDAPDESPGGVLNAYQLTNPLES
FAKLGKQTGILFYVPAAYTSKIDPTTGFVNLFNTSSKTNAQERKEFLQKFESISYSAKDGGIFAFAFDYRKFGTSKT
DHKNVWTAYTNGERMRYIKEKKRNELFDPSKEIKEALTSSGIKYDGGQNILPDILRSNNNGLIYTMYSSFIAAIQMR
VYDGKEDYIISPIKNSKGEFFRTDPKRRELPIDADANGAYNIALRGELTMRAIAEKFDPDSEKMAKLELKHKDWFEF
MQTRGD

### Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) or polypeptide is free of the amino acid sequences or nucleic acid sequences that flank it in its naturally-occurring state. Purified also defines a degree of sterility that is safe for administration to a human subject, e.g., lacking infectious or toxic agents

Relative to a control level, the level that is determined may an increased level. As used herein, the term “increased” with respect to level (e.g., cytokine release, gene regulation, or metabolic rate of T cells after the described SOLUPORE™ methods) refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., cytokine release, gene regulation, or metabolic rate of T cells after the described SOLUPORE™ methods) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

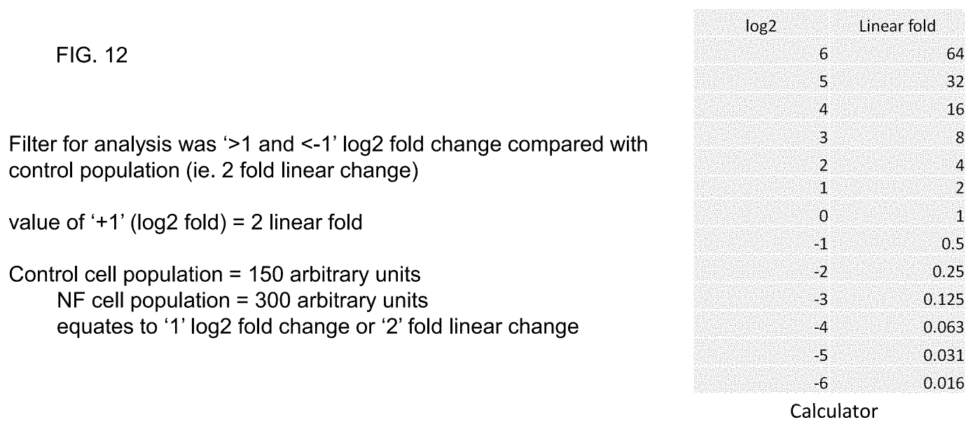

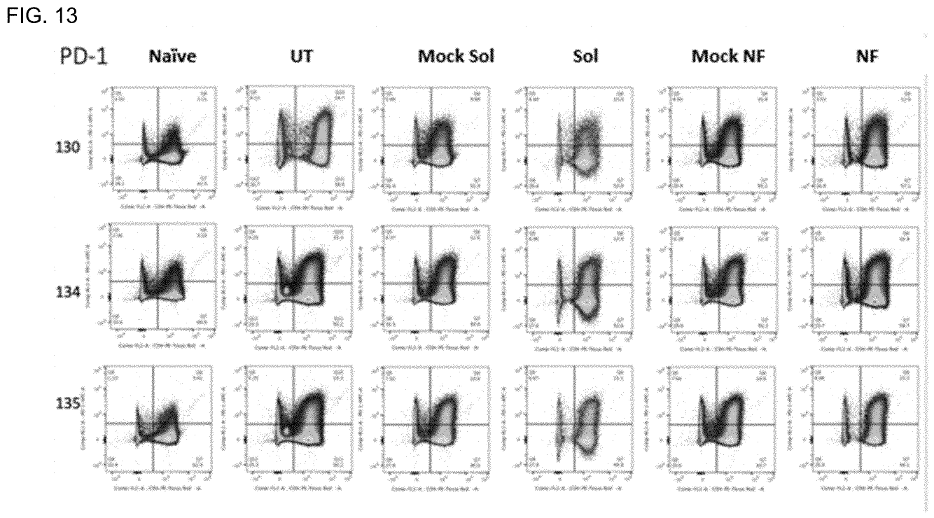

The increase or decrease may also be expressed as fold-difference or log-difference (see, e.g., FIG. 12 for correlation). For example, Log base 2 (or log₂) was used to normalize the results along an axis with equal values for upregulated and downregulated genes. An exemplary calculation is shown below:

gene A treated vs control=7.0 (overexpressed);

gene B control vs treated=7.0 or treated vs control=0.142 (underexpressed).

Both are overexpressed or underexpressed with the same intensity but in a linear scale this is not reflected. Alternatively, gene A is 7.0 fold up and gene 2 is 0.142 down regulated. If this is expressed in log₂ then gene A is 2.81 fold upregulated and gene B is −2.81 fold downregulated.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: Efficient and Versatile Engineering of Primary Human Immune Cells

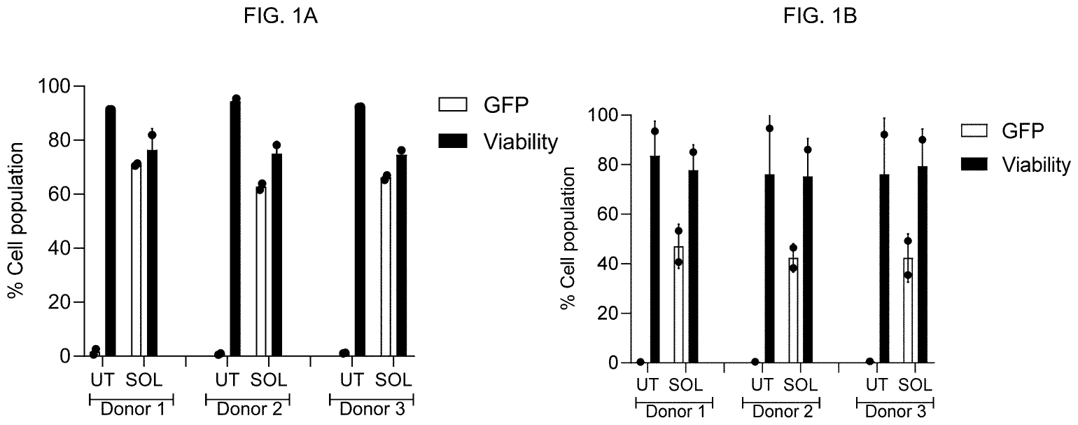

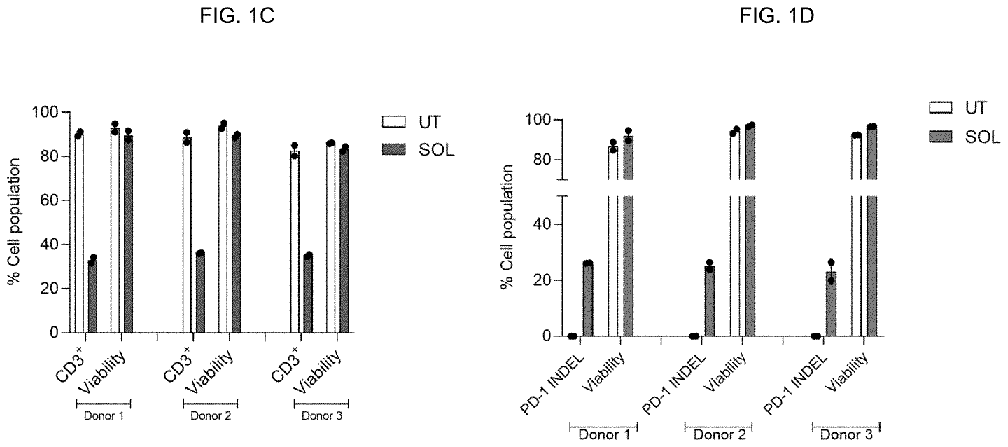

The ability of the SOLUPORE™ delivery method to deliver a model cargo, GFP (green fluorescent protein) mRNA, to primary human T cells was evaluated. Because T cell therapy manufacturing processes are diverse and include a variety of cell culture regimes, both PBMC (Peripheral Blood Mononuclear Cells)-initiated- and CD3+ (cluster of differentiation 3) purified T cell cultures were used, each isolated from three human donors. GFP expression at 24 hr was 65-75% and 40-50% for PBMC-initiated- and CD3+ purified T cells respectively with cell viabilities greater than 70% (FIG. 1A and FIG. 1B).

Next the SOLUPORE™ delivery method efficiency was assessed with functional cargos using Cas9 (CRISPR-associated endonuclease Cas9 (Cas9)) protein-gRNA ribonucleoprotein (RNP) complexes designed to target the TRAC (T-cell receptor alpha) and PDCD1 (programmed death cell protein 1) genes. RNPs were delivered to T cells isolated from three donors. With the TRAC RNPs, CD3 expression was reduced from 90% to 35% with corresponding cell viability >90% (FIG. 1C). For PDCD1, INDEL (insertion or deletion of bases) efficiencies of 25% were achieved with >90% cell viability (FIG. 1D).

### Example 2: Dual and Sequential Delivery of Multiple Cargos

Next generation immune cell therapy products will require several modifications meaning that transfection technologies will be required to deliver multiple cargos. However, such engineering is only useful if cell health and functionality are not adversely impacted by the delivery method. Thus, the SOLUPORE™ delivery method was evaluated to deliver two cargos, either simultaneously or in sequence. The maintenance of the cell viability was also evaluated.

### Dual Cargo Delivery

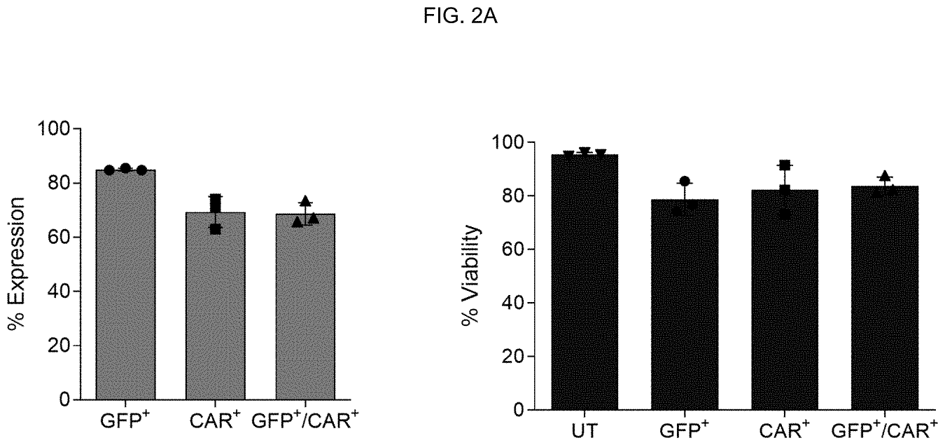

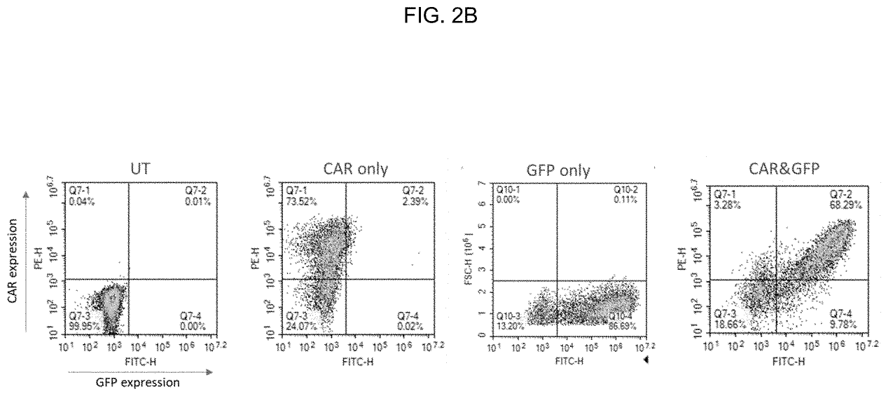

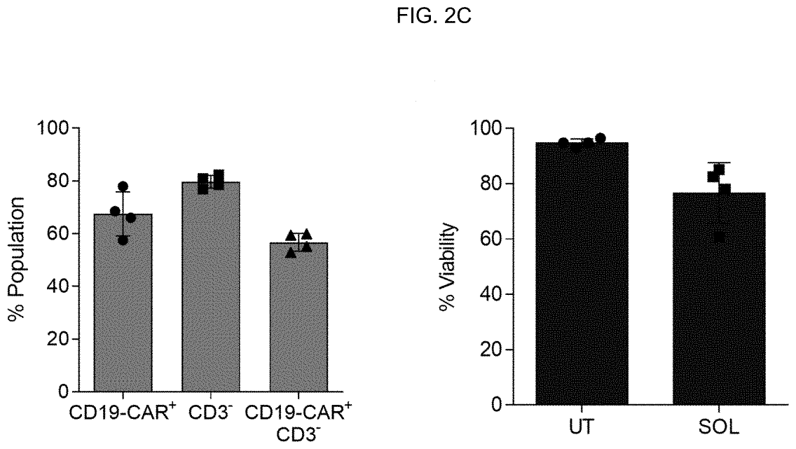

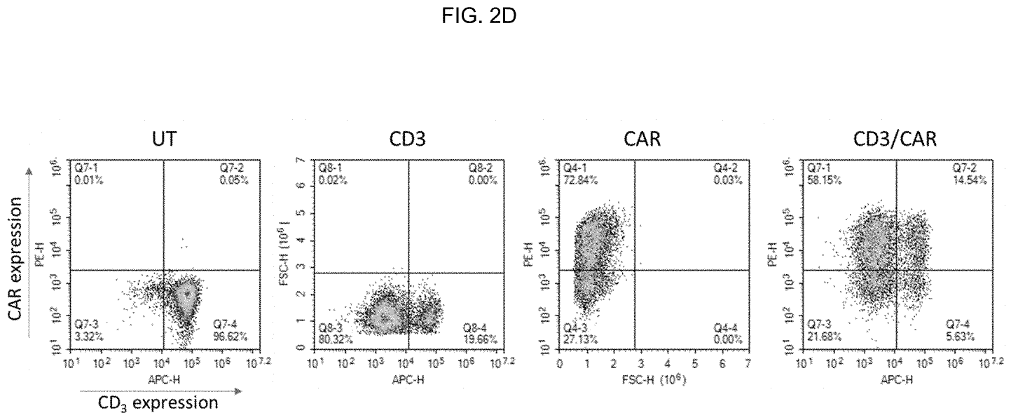

To test the concept of dual cargo delivery, CD19 (cluster of differentiation 19) CAR (chimeric antigen receptor) mRNA and GFP mRNA were delivered simultaneously to stimulated T cells from 3 donors by either by the SOLUPORE™ delivery method or electroporation. At 24 hr post-transfection, 68.7±4.1% of the population of cells using the SOLUPORE™ delivery method were both CD3 positive and CAR positive, and cell viability remained high (FIG. 2A). Representative flow cytometry plots are shown in FIG. 2B.

Delivery of multiple cargos provides therapeutic advantages, wherein multiple or complex cargos are required for effective treatment.

Delivery of multiple cargos enables complex editing to be carried out on cells. Each cargo can endow a specific function or feature to a cell. If targeting and efficacy are to be enhanced in autologous cell therapies for both liquid and solid tumors, cells will require multiple modifications using steps that are aligned with manufacturing processes. This may involve multiplex or sequential engineering steps. Similar demands apply to allogeneic approaches where cell rejection and GvHD issues mean that complex editing is likely to be required. Limitations in viral vector capacity and electroporation toxicity mean that these modalities may be unsuitable for certain complex engineering regimes. Moreover, the long lead time required to design and generate even research grade viral vectors means that timelines may be longer than desired at the development stage. This is of particular concern in relation to progressing approaches for solid tumors where targeting and efficacy challenges mean that large numbers of candidate target antigens and cell potency enhancements will need to be tested. It will be necessary to evaluate a myriad of cell compositions in a rapid, high-throughput fashion that is likely to be highly constrained if wholly reliant on viral vectors.

### Sequential Cargo Delivery

To assess sequential delivery, TRAC (T-cell receptor alpha) RNP (ribonucleoprotein) was delivered to T cells and two days later, CD19 CAR mRNA was delivered to the same population of cells. The following day, cells were harvested and analysed for CD3 and CAR expression. CAR expression averaged 67.5±8.4%, CD3 knockdown was 79.7±2.4% and 56.7±3.4% of cells were both CAR-positive and CD3-negative (FIG. 2C). The average viability of cells was 76.7±10.9% while untreated control cells were 94.74±4.5%. Representative flow cytometry plots are shown in FIG. 2D. Sequential delivery, as with dual/multiplex delivery of cargos provides therapeutic advantages, wherein cells can be modified to possess multiple new features that enhance their ability to target tumor cells or to effectively kill the tumor cells. For example to enhance targeting it may be necessary to target multiple tumor antigens. Additionally, to augment T cell trafficking there is interest in expressing chemokine receptors or cytokines on CAR-T cells, or expressing stroma-degrading enzymes to augment CAR-T cell migration through the tumor, or enhancing persistence by expressing dominant/negative forms of CAR-T inhibitors such as PD-1 and TGFβ, and many other strategies.

### Example 3: Cytokine Release Demonstrated Minimal Cell Perturbation

The cargo delivery studies in Example 2 above demonstrated that transfection with the SOLUPORE™ delivery method is efficient while having a minimal effect on cell viability. However, it has been reported that delivery methods such as electroporation can minimally affect T cell viability yet can cause stress to cells that causes unintended changes to gene and protein expression and ultimately cell functionality. Thus, the effect of transfection on cytokine release and immune gene expression in T cells (Example 4) was evaluated.

It was first examined whether the SOLUPORE™ delivery method caused non-specific release of cytokines from T cells using a multiplex assay (Luminex). The panel contained 11 human analytes: IFN-γ (interferon gamma), IL-2 (interleukin 2), TNFα(tumor necrosis factor alpha), IL-8 (interleukin 8), GM-CSF (Granulocyte-macrophage colony-stimulating factor), IL-10 (interleukin 10), MIP-1α(macrophage inflammatory protein 1 alpha), MIP-1β (macrophage inflammatory protein 1 beta), IL-17A (interleukin 17A), Fractalkine, and ITAC (Interferon—inducible T Cell Alpha Chemoattractant).

GFP mRNA was delivered by soluporation to stimulated T cells from 5 donors, with 2 technical repeats included for each donor. Mock transfections (without cargo) were also included and cytokine release was measured over a 5-day time course. No significant difference was seen with the SOLUPORE™ delivery method GFP mRNA and mock transfection groups were compared with untreated control cells for any of the cytokines analysed. This indicated that the cells were not perturbed in such a way to cause non-specific release of these cytokines.

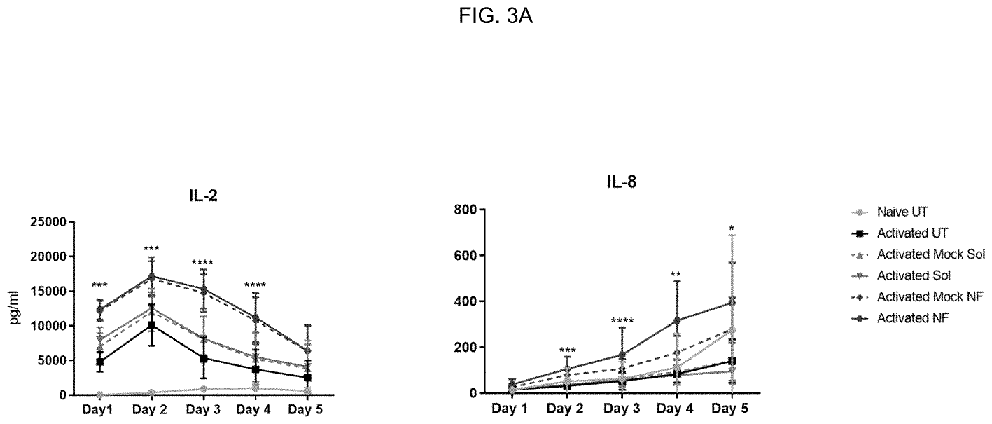

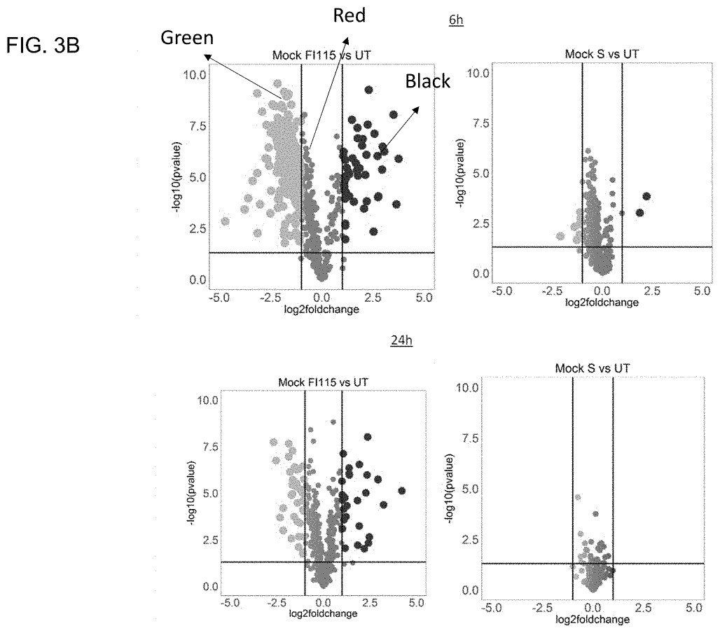

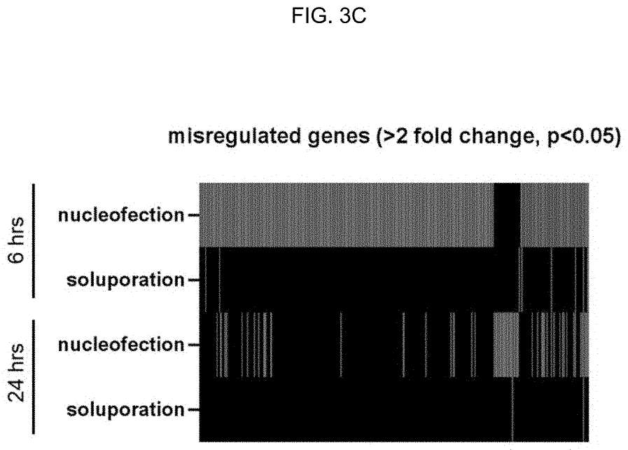

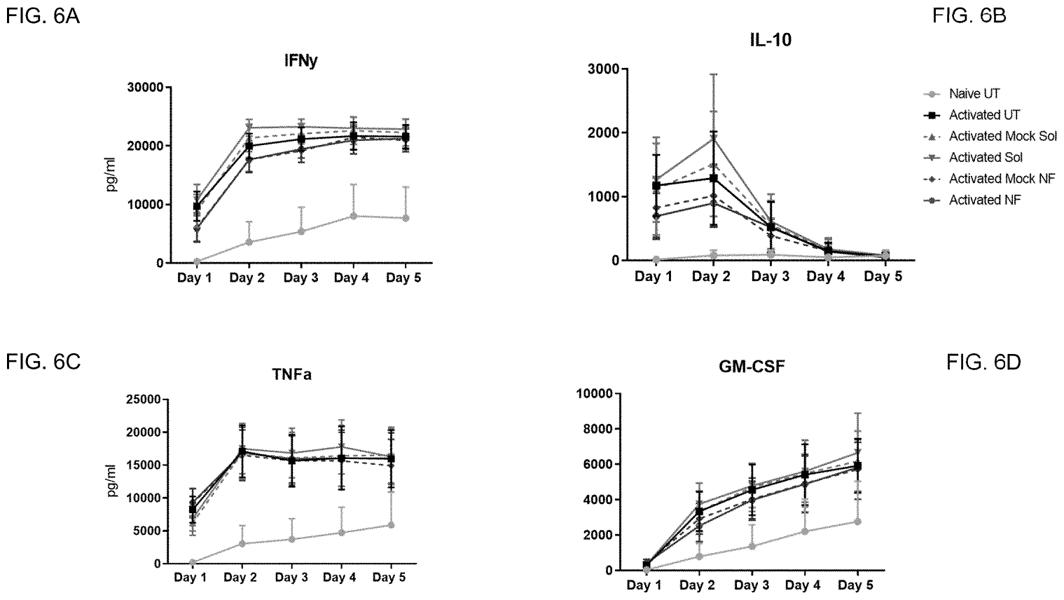

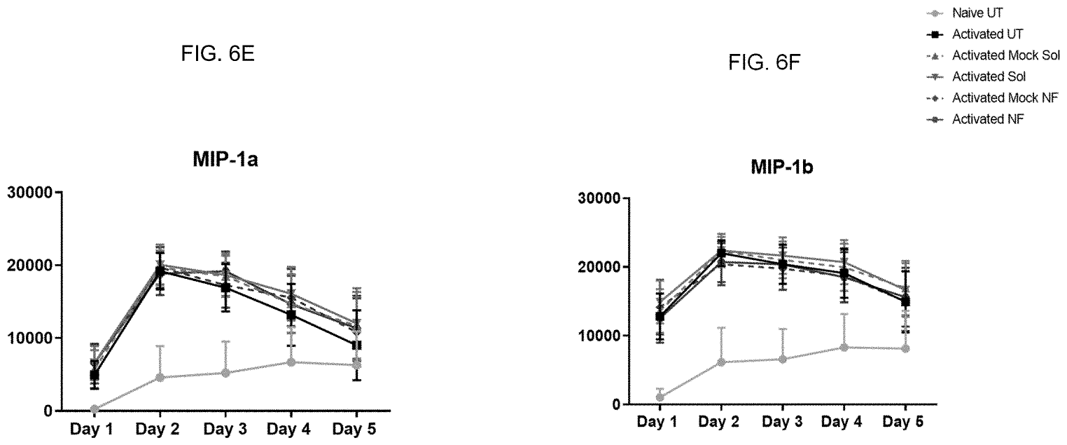

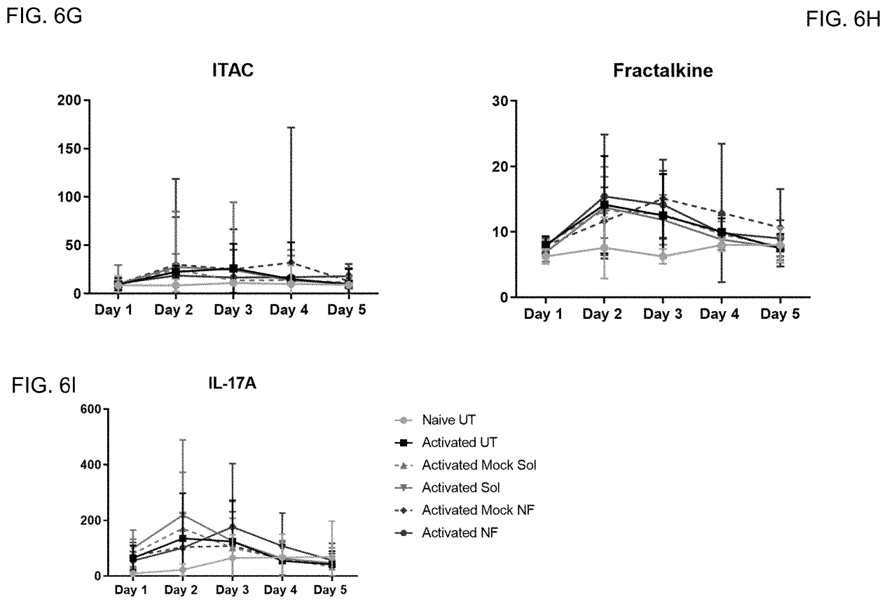

In contrast, when electroporation was used to transfect cells, significant differences in secretion of IL-2 and IL-8 were evident suggesting that the electroporation process caused cell stress that led to cytokine release from these cells (FIG. 3A and FIGS. 6A-6I). T cells release cytokines either specifically, in response to specific stimulatory ligands, or non-specifically in response to stress. No specific stimulatory ligands were used in these experiments, indicating that the cells that were electroproated were stressed.

### Example 4: Immune Gene Profiling Demonstrated Minimal Cell Perturbation

The experiments described herein were performed in a cargo-independent manner, meaning they were performed to demonstrate that the SOLUPORE™ delivery method had minimal impact on protein and gene expression in T cells, and importantly, biological attributes such as proliferation were preserved. Furthermore, the addition of exogenous cargo to the immune cell using the SOLUPORE™ delivery method will have a minimal impact on protein and gene expression, and will also maintain biological function and activity. The impact of the transfection processes on gene expression in T cells using the Nanostring CAR-T Characterisation panel which measures the gene expression of up to 780 immune-related genes including genes relevant to immune cell exhaustion, activation and persistence was evaluated. It has been reported that electroporation can dramatically affect gene expression in T cells. Unstimulated T cells (or “unactivated T cells”) that were mock transfected were used to avoid potential confounding effects of the cargo on gene expression. The “high efficiency for T cells” FI-115 electroporation program was used recommended by the manufacturer (Lonza).

In the first of these studies (Study 1), unactivated T cells from 3 donors, each with two technical repeats included, were mock transfected using either the SOLUPORE™ delivery method or electroporation. Gene expression was analysed at 6 hr and 24 hr post-transfection. In the 6 hour group of the SOLUPORE™ delivery method, 1.7% of genes were identified as differentially expressed group (10/582 genes, 1 log 2 fold (>2 fold) change, p<0.05, Tables 7-8), compared with untreated control cells. At 24 hr post-transfection (of the SOLUPORE™ delivery method), no changes in gene expression were identified (0/582 genes).

In contrast, for the electroporation 6 hr group, 265/582 genes were identified as changed, representing 45.5% of the genes detected (Tables 7-9). In the 24 hr electroporation group, 11.3% of genes were differentially expressed (66/582, Table 9). When the 6 hr and 24 hr electroporation groups were compared, 37 genes were found to be differentially expressed at both timepoints (Table 4, below).

TABLE 4
Study 1 - For electroporation, 37 genes were
common at the 6 hr and 24 hr timepoint

| Gene name | 6 hr | 24 hr |
|---|---|---|
| BATF3 | 2.72 | 2.93 |
| BATF | 1.22 | 1.38 |
| CCL4/L1 | 1.84 | −1.71 |
| CD200 | 5.51 | 1.93 |
| CD68 | −1.09 | 1.09 |
| CTSW | −2 | −1.87 |
| CX3CR1 | −1.98 | −2.68 |
| CXCL10 | 2.06 | 1.16 |
| FASLG | 2.18 | −1.41 |
| FCGR3A/B | −2.03 | −2.54 |
| FOSB | 6.15 | 3.23 |
| FOS | 3.05 | 1.84 |
| GZMA | −2.6 | −1.66 |
| GZMH | −1.64 | −1.8 |
| GZMK | −2.11 | −1.68 |
| IFIT3 | 2.74 | 1.92 |
| IL12RB2 | 1.13 | 1.83 |
| IL2 | 3.64 | 1.11 |
| IL7R | −1.39 | −1.34 |
| IRF4 | 3.5 | 2.37 |
| JUN | 1.56 | 2.34 |
| KIR3DL1/2 | −2.15 | −1.25 |
| LAIR1 | −2.31 | −1.66 |
| MTHFD1L | 1.15 | 1.02 |
| NFIL3 | 1.71 | 1.01 |
| NFKBIA | 2 | 1.09 |
| PECAM1 | −2.34 | −1.12 |
| PRF1 | −2.04 | −1.46 |
| RPTOR | −1.09 | 1.02 |
| SELL | −1.15 | −1.19 |
| SLC3A2 | 1.28 | 1.06 |
| SLC7A5 | 3.75 | 1.26 |
| TIMP1 | −2.49 | −1.43 |
| TRGC1 | −1.15 | −1.1 |
| TRGV8 | −1.29 | −1.65 |
| TYROBP | −1.97 | −1.39 |
| XCL1/2 | 1.33 | −1.09 |

Of the 10 genes identified in the 6 hour group of the SOLUPORE™ delivery method, 8 genes were common with the electroporation 6 hr group (Table 5, below).

TABLE 5
Common genes at 6 hr in electroporation
and the SOLUPORE ™ delivery
method groups in Study 1

| Gene name | Electroporation | SOLUPORE ™ delivery method |
|---|---|---|
| CD160 | 1.06 | −2.09 |
| FOSB | 6.15 | 2.21 |
| IFIT3 | 2.74 | 1.88 |
| IL2 | 3.64 | −1.15 |
| JUN | 1.56 | −1.08 |
| SGO2 | −3.16 | −1.13 |
| TRAV1-1 | −2.51 | −1.38 |
| TRBV30 | −1.84 | −1.27 |

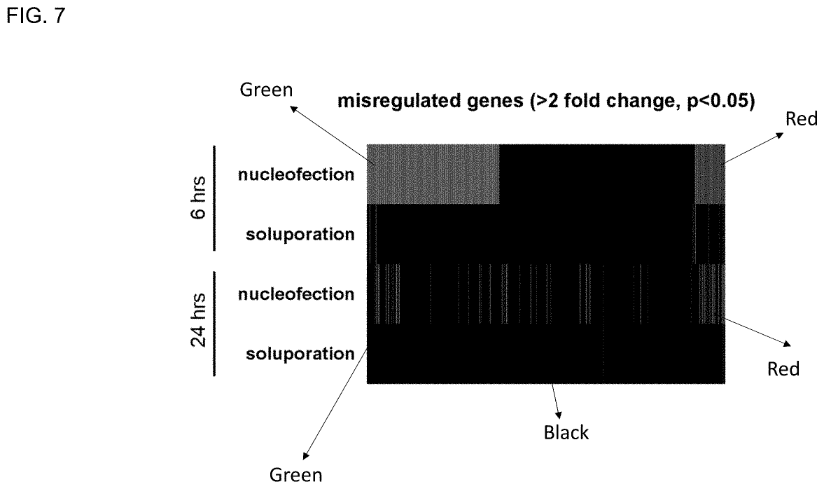

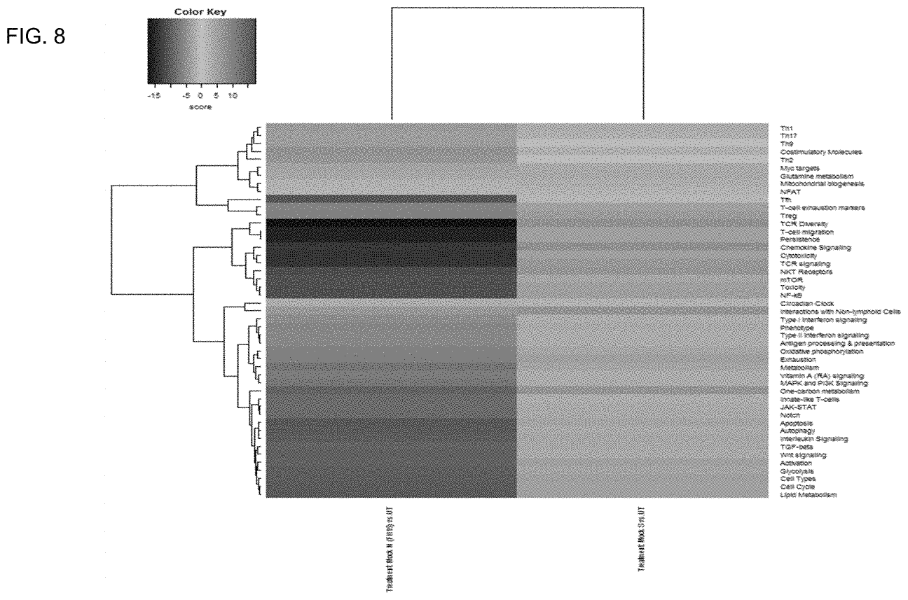

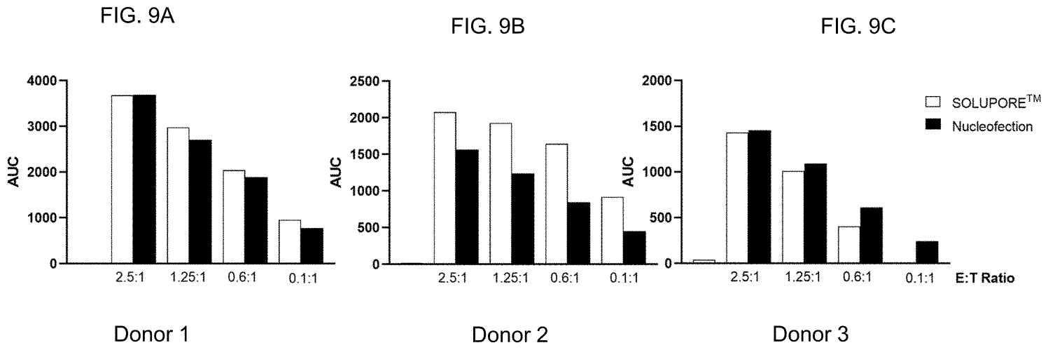

Volcano plots (FIG. 3B) and heat maps (FIG. 3C and FIG. 7) were generated to provide an overview of differentially expressed genes. A pathway analysis was also completed (Table 1, below and FIG. 8). A majority of the genes identified in the electroporation 6 hr group mapped to pathways associated with T cell activation, metabolism and exhaustion.

TABLE 1
Pathway analysis of genes identified at
6 hr in CAR-T Characterisation Panel

| Pathway | No. genes identified | No. genes identified |
|---|---|---|
| (No. associated | in electroporation- | in SOLUPORE ™ delivery |
| genes in panel) | treated cells | method-treated cells |
| Activation (200) | 77 | 4 |
| Metabolism (193) | 56 | 2 |
| Exhaustion (103) | 49 | 2 |
| TCR signaling (48) | 25 | 3 |
| Apoptosis (48) | 22 | 1 |
| Chemokine | 15 | 1 |
| signaling (22) | | |
| T cell migration and | 11 | 0 |
| persistence (24) | | |
| Glycolysis (19) | 8 | 0 |
| Antigen processing | 6 | 0 |
| and presentation | | |
| (27) | | |

Given the large number of gene changes in the electroporation group, a second study was performed, in order to validate the findings. In Study 2, gene expression was analysed at 24 hr post-transfection. Each group included unstimulated T cells from 2 donors, with 2 technical repeats and a third donor done once, all mock transfected. The results were similar to those seen in the first study with only 9/597 genes (1.5%) identified for the SOLUPORE™ delivery method and 43/597 (7.2%) genes for the FI-115 electroporation group (Tables 11 and 12), showing consistency with Study 1. Four genes were identified as common between the SOLUPORE™ delivery method and electroporation 24 hr groups in this study (Table 6, below).

TABLE 6
Common genes at 24 hr in electroporation
and the SOLUPORE ™ delivery
method groups in Study 2

| | | SOLUPORE ™ |
|---|---|---|
| Gene name | Electroporation | delivery method |
| BATF3 | 2.05 | 1.08 |
| FOSB | 2.82 | 1.14 |
| IFIT3 | 1.99 | 1.77 |
| TNFRSF11A | 2.44 | 1.08 |

When Study 1 and Study 2 were compared, 38 genes were found to be common in the 24 hr electroporation groups, again showing consistency between the studies (Table 2, below).

TABLE 2
Comparison of Study 1 and Study 2 electroporation groups
at 24 hr timepoint showing common genes identified
with 1 log2 fold (>2 fold) change, p < 0.05.

| Gene name | Study 1 | Study 2 |
|---|---|---|
| AHR | 1.38 | 1.29 |
| BATF | 1.38 | 1.23 |
| BATF3 | 2.93 | 2.05 |
| CCL22 | 4.21 | 2.09 |
| CCL4/L1 | −1.71 | −1.42 |
| CD19 | 1.57 | 1.58 |
| CD200 | 1.93 | 2.92 |
| CD244 | −1.31 | −2.26 |
| CD38 | 2.93 | 2.33 |
| CD68 | 1.09 | 1.85 |
| CTSW | −1.87 | −1.64 |
| CX3CR1 | −2.68 | −2.12 |
| FCGR3A/B | −2.54 | −1.4 |
| FOS | 1.84 | 1.82 |
| FOSB | 3.23 | 2.82 |
| GZMA | −1.66 | −1.36 |
| GZMH | −1.8 | −1.08 |
| GZMK | −1.68 | −1.54 |
| ICOSLG | 1.21 | 1.27 |
| IFIT3 | 1.92 | 1.99 |
| IL12RB2 | 1.83 | 1.46 |
| IL7R | −1.34 | −1.04 |
| IRF4 | 2.37 | 1.81 |
| IRF8 | 2.28 | 1.94 |
| ITGAM | −1.86 | −1.64 |
| JUN | 2.34 | 1.96 |
| KLRB1 | −1.55 | −1.23 |
| LAIR1 | −1.66 | −1.2 |
| MT2A | 1.39 | 1.14 |
| NCR1 | −1.72 | −1.28 |
| NFIL3 | 1.01 | 1.11 |
| NT5E | −1.43 | −1.22 |
| PRF1 | −1.46 | −1.14 |
| SELL | −1.19 | −1.05 |
| TIMP1 | −1.43 | −1.42 |
| TRGC2 | −1.27 | −1.03 |
| TRGV2 | −1.45 | −1.12 |
| TYROBP | −1.39 | −1.4 |

An additional nucleofection program, EO-115, was also included in Study 2. This program is described by the manufacturer as “high cell functionality” and is presumably less harsh than FI-115. With the EO-115 program, 16/597 (2.7%) genes were differentially expressed (Tables 11 and 12). There was a high degree of overlap in the genes identified in the two nucleofection programs with 12 of the 16 genes in the EO-115 group also present in the FI-115 group (Tables 11 and 12). The lower number of genes identified in the EO-115 group compared with FI-115 was consistent with this being a less harsh electroporation program.

### T Cell Exhaustion Characterization and Phenotype

For next generation CAR T therapies, several issues were examined with a view for achieving long term disease control in greater numbers of patients and improving responses in solid tumors and T cell exhaustion is receiving increasing attention in this regard. The T cell exhaustion phenotype occurs naturally following prolonged antigen exposure during chronic viral infections or cancer and is characterised by expression of inhibitory receptors, metabolic impairment and down-modulation of effector function such as cytokine secretion. It has been suggested that exhaustion involves substantial rewiring of TCR (T cell receptor) signaling-mediated metabolic process and that transcription factors including AP-1 (activator protein 1) complexes, IRF4 (Interferon regulatory factor 4), BATF (Basic leucine zipper transcription factor, ATF-like) and NFAT (Nuclear factor of activated T-cells) play key roles in this process.

While antigen signaling through the T cell receptor leads to activation of these signaling pathways, cellular stresses can also stimulate these pathways in T cells. Therefore, exhaustion-related genes in the CAR-T characterisation panel were evaluated. Expression of FOSB (Fos proto-oncogene), FOS (proto-oncogene), JUN, BATF (Basic leucine zipper transcriptional factor ATF-like), BATF3 (Basic leucine zipper transcriptional factor ATF-like 3) and IRF4 (Interferon regulatory factor 4) genes were consistently upregulated in the electroporation groups across both studies, whereas there was minimal perturbation of these genes using the SOLUPORE™ delivery method groups compared with untreated control cells (Table 3, below).

TABLE 3
AP-1 (activator protein 1)-related genes identified with 1 log2 fold
(>2 fold) change, p < 0.05 in Study 1 and Study 2

| | SOLUPORE ™ delivery | | | | | | |
|---|---|---|---|---|---|---|---|
| | method vs Control | | | Electroporation vs Control | | | |
| | | | | Study 1 | | Study 2 | |
| | Study 1 | | Study 2 | 6 hr | 24 hr | 24 hr | 24 hr |
| Gene | 6 hr | 24 hr | 24 hr | FI-115 | FI-115 | FI-115 | EO-115 |
| FOSB | 2.21 | n.i. | 1.14 | 6.15 | 3.23 | 2.82 | 2.74 |
| FOS | n.i. | n.i. | n.i. | 3.05 | 1.84 | 1.82 | 1.6 |
| JUN | −1.08 | n.i. | n.i. | 1.56 | 2.34 | 1.96 | 1.64 |
| BATF | n.i. | n.i. | n.i. | 1.22 | 1.38 | 1.23 | n.i. |
| BATF3 | n.i. | n.i. | 1.08 | 2.72 | 2.93 | 2.05 | 1.45 |
| IRF4 | n.i. | n.i. | n.i. | 3.5 | 2.37 | 1.81 | n.i. |

n.i. = not identified

In other embodiments, the immune cell of the invention (the immune cell having the exogenous cargo) has a molecular profile where programmed death protein 1 (PD1) is expressed at a level a log₂ fold change of 3, a log₂ fold change of 2, or a log 2 fold change of 1 compared to of the level expressed in a control immune cell.

Exhausted T cells display a transcriptional program distinct from that of functional effector or memory T cells, characterized by the expression of inhibitory cell surface receptors, including PD-1. For example, the immune cell of the invention (the immune cell having the exogenous cargo) has a molecular profile where PD-1 is expressed at a level about a log 2 fold change of 1 compared to the level expressed in a control immune cell). For example, the immune cell of the invention (the immune cell having the exogenous cargo) has a molecular profile where PD-1 is expressed at a level about a log₂ fold change of 2 compared to the level expressed in a control immune cell. For example, the immune cell of the invention (the immune cell having the exogenous cargo) has a molecular profile where PD-1 is expressed at a level about a log₂ fold change of 3 compared to the level expressed in a control immune cell. In some embodiments, the immune cell of the invention (the immune cell having the exogenous cargo) has a molecular profile where PD1 is expressed at about a log₂ fold change of −3, a log₂ fold change of −2, or a log₂ fold change of −1 compared to the level expressed in a control immune cell.

### Example 5: Proliferation and In Vivo Engraftment of Transfected Cells

Taken together, the cargo delivery and gene and protein expression studies described above indicate that the SOLUPORE™ delivery method can efficiently deliver cargo to modify T cells causing minimal cell stress and nonspecific perturbation of protein and gene expression. However, for cell therapy manufacturing applications, it is also necessary to confirm that the modified cells retain their desired biological attributes such as robust proliferation and in vivo engraftment. Therefore these features were examined in transfected T cells.

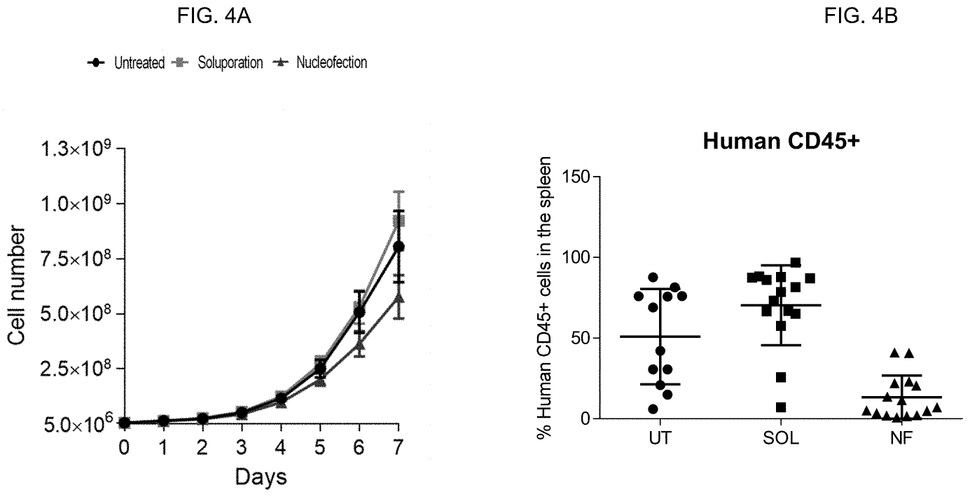

To examine the effect of transfection on T cell proliferation, cells isolated from 5 random donors were transfected with GFP mRNA. Each donor included 5 independent technical repeats and cell proliferation was counted over 7 days. The proliferation rate of cells transfected with the SOLUPORE™ delivery method was similar to untreated control cells (FIG. 4A). In contrast, cells transfected using electroporation proliferated at a slower rate.

To further assess the impact of transfection on T cell health and functionality, an in vivo engraftment mouse model was used. Humanised mouse models of xenogeneic-GvHD based on immunodeficient strains injected with human peripheral blood mononuclear (hu-PBMC) are important tools for studying human immune function. The model is characterised by the engraftment of hu-PBMC in the blood and ultimately in the spleen, lymph nodes and bone marrow of injected mice. These cells readily engraft following intravenous injection in immunodeficient NOD/SCID/γ^{/-} (NSG) mice that lack T, B and NK cells and bear a targeted mutation of the IL-2 receptor gamma chain (IL-2Rγnull) which permits acceptance of human cells and tissues. Successful engraftment and development of GvHD is dependent on hu-PBMC reactivity with mouse MHC class I and II and therefore relies upon highly viable and functional donor cells.

Human PBMC were transfected with 3 kDa dextran-Alexa Fluor 488 using the SOLUPORE™ delivery method or electroporation and infused into irradiated NOD-scid IL-2Rγnull mice. Upon harvest at day 28 post-injection, the cells using the SOLUPORE™ delivery method were found to have engrafted in the spleen at levels similar to untreated control cells (FIG. 4B). In contrast, electroporated cells exhibited low levels of engraftment indicating reduced functional efficacy in these cells.

### Example 6: Generation of CD19 CAR-T Cells and In Vitro and In Vivo Cytotoxicity

Having demonstrated that the SOLUPORE™ delivery method allows T cells to be efficiently modified whilst retaining their proliferation and engraftment capacity (Examples 2-5 above), CAR-T cells were generated and their cancer cell killing ability in vitro and in vivo was evaluated.

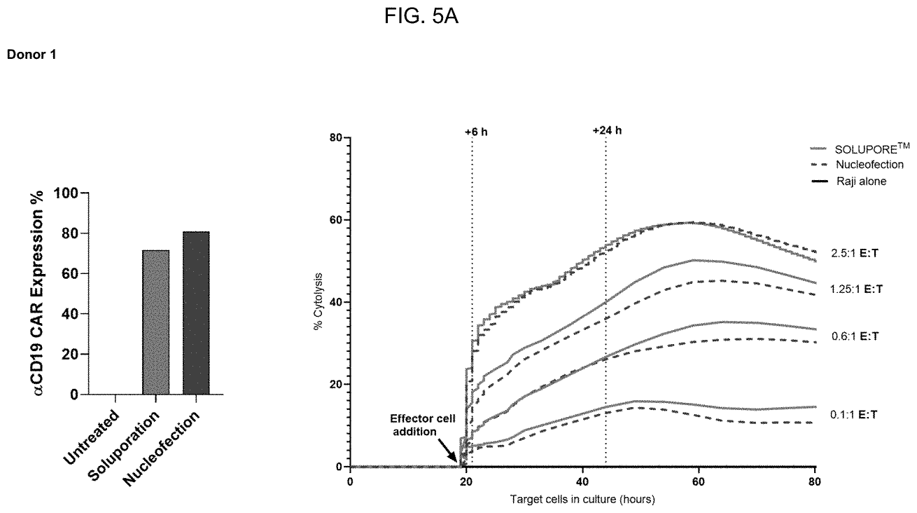

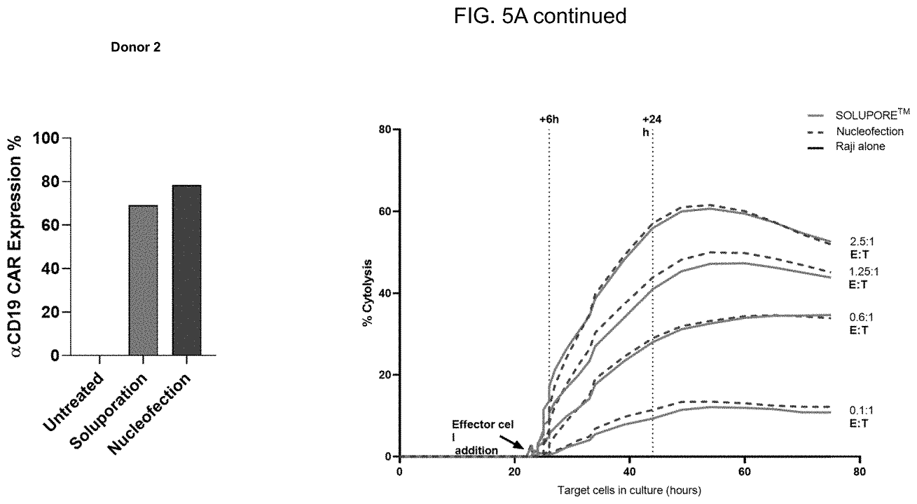

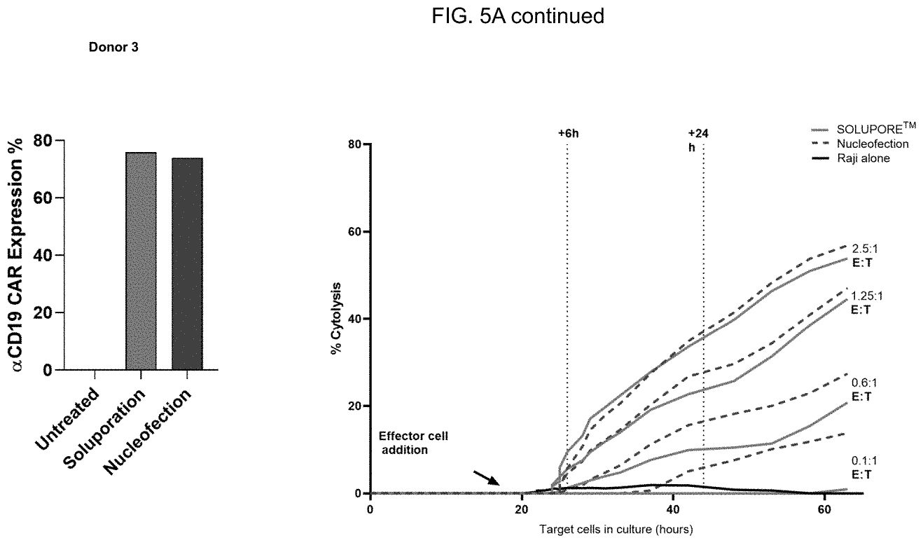

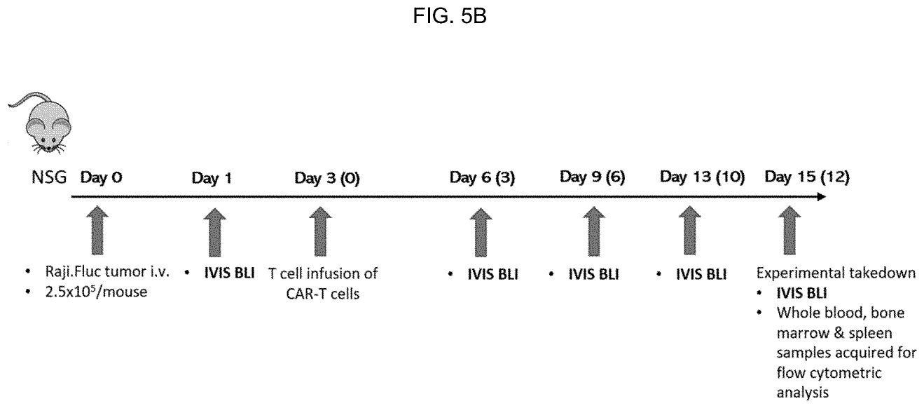

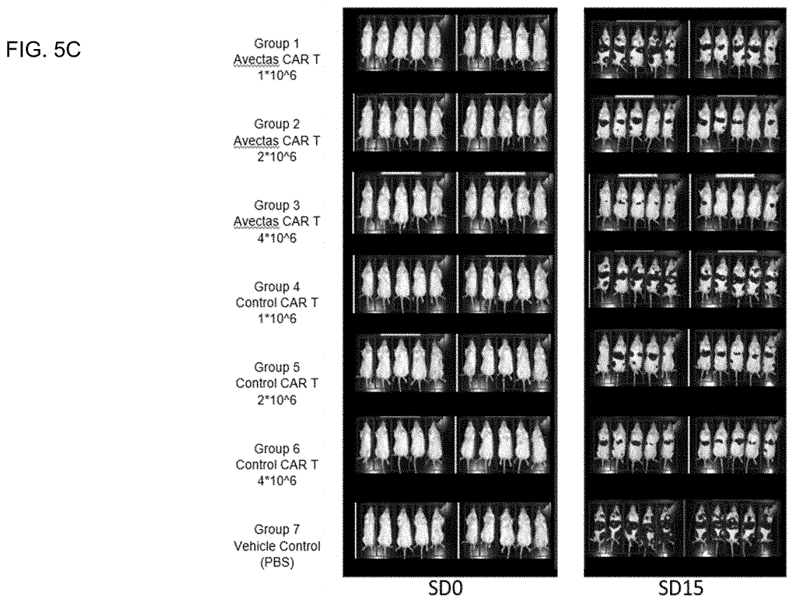

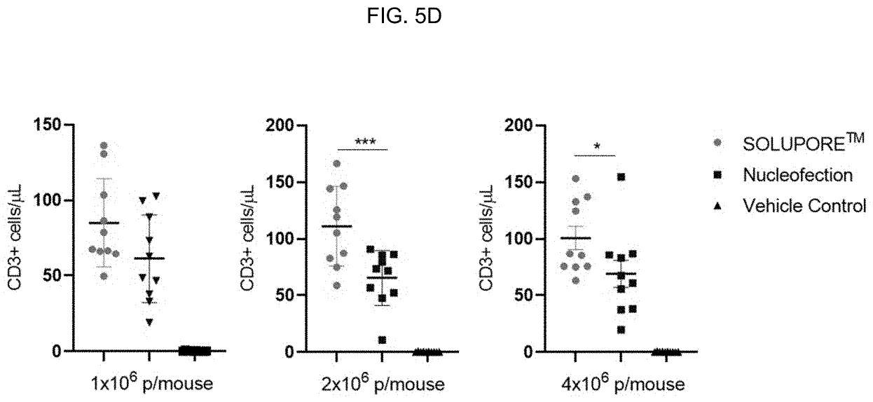

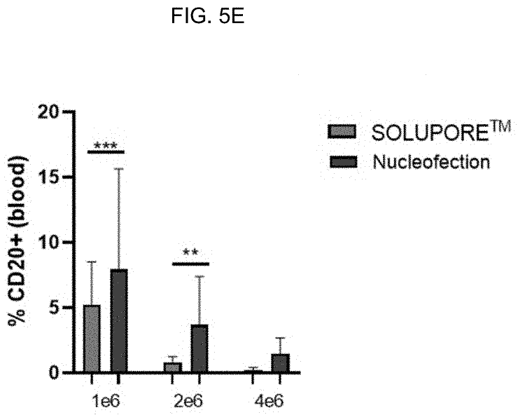

CD19 CAR mRNA was delivered to T cells from 3 donors using either the SOLUPORE™ delivery method or electroporation. CD19 CAR expression was slightly lower using the SOLUPORE™ delivery method compared with electroporated cells ranging from 72-76% and 74-81% respectively across the 3 donors (FIG. 5A). In vitro cytotoxicity against CD19-expressing RAJI cells was determined using a real-time cellular impedance assay. CAR-T cells using the SOLUPORE™ delivery method showed equivalent cytotoxicity with electroporated CAR-T cells against the target RAJI cells despite having lower levels of CAR expression (FIG. 5A).

The in vivo therapeutic potential using the SOLUPORE™ delivery method generated CAR T cells was evaluated using a luciferase-expressing RAJI tumor model in NSG mice (FIG. 5B). CD19 CAR T cells were generated using the SOLUPORE™ method, and electroporation was used as a positive control; the average CAR expression was 73% and 85% CAR respectively. Mice received doses of 1×10⁶, 2×10⁶ or 4×10⁶ CAR T cells and disease progression was monitored by bioluminescent imaging. At 12 days following CAR T cell dosing, reduced tumor growth was evident in a dose dependent manner using SOLUPORE™ delivery method as well as the positive control electroporation cohorts (FIG. 5D). While the reduction in tumor burden was similar between the respective SOLUPORE™ delivery method and electroporation doses, it was notable that 3/10 mice in the highest dose, 4×10⁶ CAR T cells, group appeared disease free. This observation correlated with the presence of significantly more human T cells in the blood of mice that received the SOLUPORE™ delivery method CAR-T cells compared with electroporation control groups as confirmed by flow cytometric analysis (FIG. 5D). Similarly, tumor engraftment, identified based on expression of CD20 (cluster of differentiation 20), was lower in those mice receiving the SOLUPORE™ delivery method prepared CAR-T cells across each of the doses tested (t-test) (FIG. 5E).

### Example 7: Phenotypic Analysis of Activated Human T Cells Following Transfection

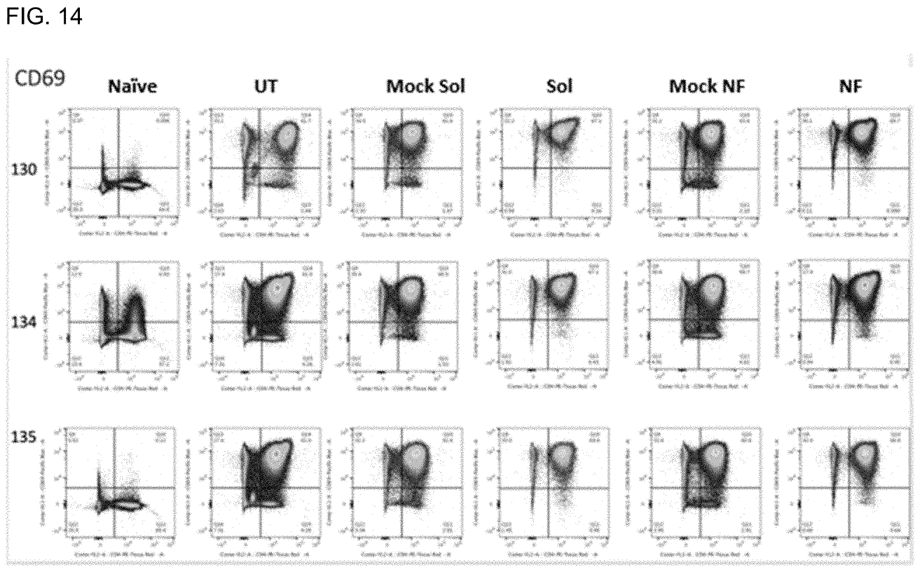

Activated CD3+ T cells from 3 donors were either Soluporated or Nucleofected (using program EO115—“high functionality for T cells”) with mRNA-GFP or in the absence of cargo (mock). The cells were analysed using a panel of monoclonal antibody (mAbs) specific for T cell related activation/exhausted surface markers (PD-1 and CD69 being the primary targets) (FIG. 13 and FIG. 14).

Across 3 donors the CD4⁺ population accounts for 65%, and cluster of differentiation 8 (CD8) (cluster of differentiation 4 (CD4) negative staining) accounts for 35% of the T cell population, both in naïve and activated UT cells i.e. a CD4:CD8 ratio of 65:35. The CD4:CD8 ratio of T cells is maintained following soluporation with GFP (67:33) or mock soluporation (63:37). The CD4:CD8 ratio of T cells is also unchanged following nucleofection with GFP (65:35) or mock nucleofection (69:31) (FIG. 13 and FIG. 14).

In 3 donors the PD1 expression in naïve CD4+ T cells is 2%. Upon activation this increased to 18%±5%. PD1 expression following soluporation, either with GFP or mock soluporation, as 16%±4% or 14%±5% respectively. Following nucleofection, either with GFP or mock nucleofection, PD1 expression in CD4+ cells is 14%±5% or 17%±5% respectively. In the same 3 donors the PD1 expression in naïve CD8+ T cells was 1%. Upon activation PD1 on CD8+ T cells increased to is 6%±1% or 7%±1% PD1 expression following soluporation, either with GFP or mock soluporation, respectively. Following nucleofection, either with GFP or mock nucleofection, PD1 expression in CD8+ cells was 5%±2% or 6%±2% respectively (FIG. 13).

In 3 donors the CD69 expression in naïve CD4+ T cells was 2%±4%. CD69 expression was upregulated upon activation to 61%±1%. CD69 expression following soluporation, either with GFP or mock soluporation, as 66%±1% or 62%±1% respectively. Following nucleofection, either with GFP or mock nucleofection, CD69 expression in CD4+ cells was 69%±2% or 62%±2% respectively. In the same 3 donors the CD69 expression in naïve CD8+ T cells was 4%±8%. Upon activation CD69 on CD8+ T cells increased to 29%±3%. CD69 expression was 32%±1% or 64%±2% following soluporation, either with GFP or mock soluporation, respectively. Following nucleofection, either with GFP or mock nucleofection, CD69 expression in CD8+ cells was 30%±2% or 32%±1% respectively (FIG. 14).

Thus, neither PD-1 or CD69 expression is altered following soluporation or nucleofection.

### Example 8: Metabolism Studies

The metabolic rate of T cells post-transfection was assessed in 3 ways: 1) production of lactate, 2) oxygen consumption rate, and 3) extracellular acidification rate. Activated T cells release lactate when they undergo metabolic remodeling from oxidative phosphorylation to aerobic glycolysis, which is required for their energetically demanding proliferation and acquisition of effector functions. Extracellular lactate correlates well with T cell proliferation. Analyzing oxygen consumption rate (OCR) and extracellular acidification rate (ECAR) gives an understanding to key cellular functions of mitochondrial respiration and glycolysis.

### Lactate Production of Activated Human T Cells Following Transfection

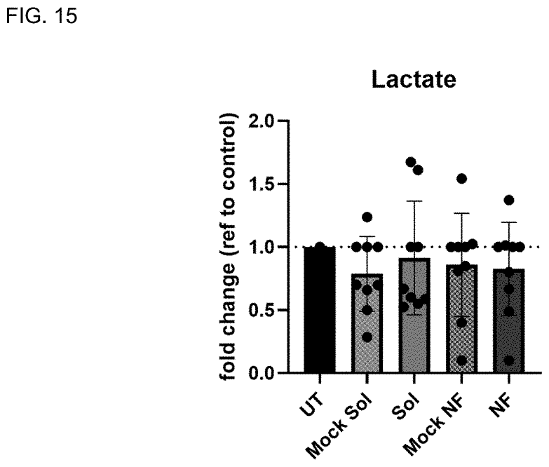

Lactate production of cells post transfection was assessed using the ChromaDazzle Lactate assay. Activated CD3+ T cells from 5 donors were either Soluporated or Nucleofected (using program EO115—“high functionality for T cells”) with mRNA-GFP or in the absence of cargo (mock). Supernatants were harvested 6 h post transfection and stored at −20° C. The ChromaDazzle Lactate assay (an enzyme-catalyzed kinetic reaction) was carried out on supernatants and the production of lactate relative to control is shown in FIG. 15.

Production of lactate from UT cells was set to 1. Compared to UT, both soluporated or nucleofected cells produce slightly less lactate, all producing between 0.8 and 0.9 times the amount of lactate as the UT (FIG. 15).

### Metabolism of Activated T Cells Following Transfection

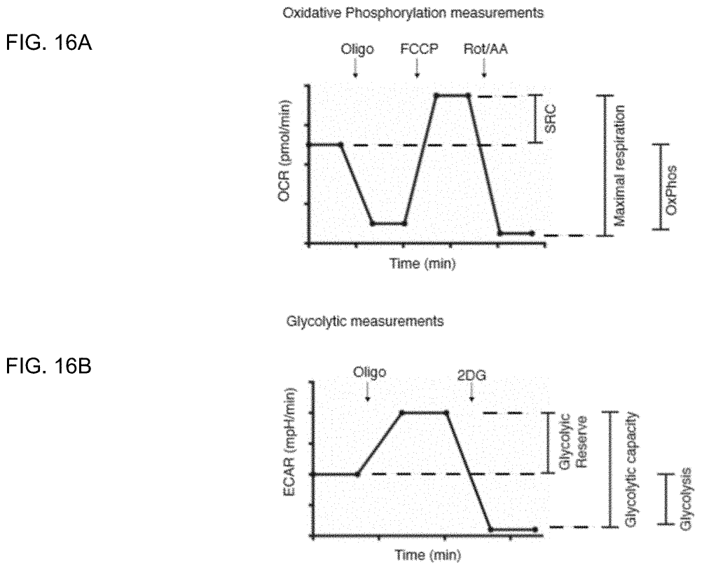

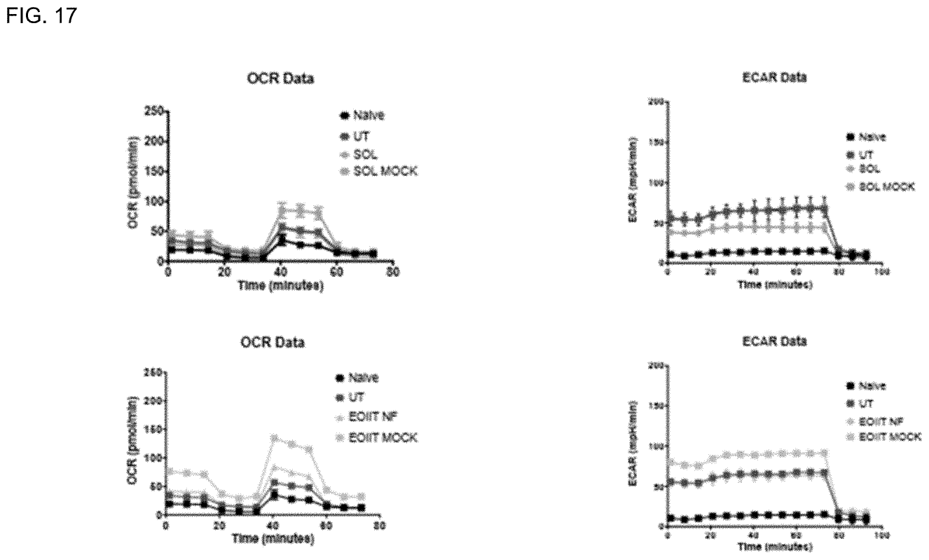

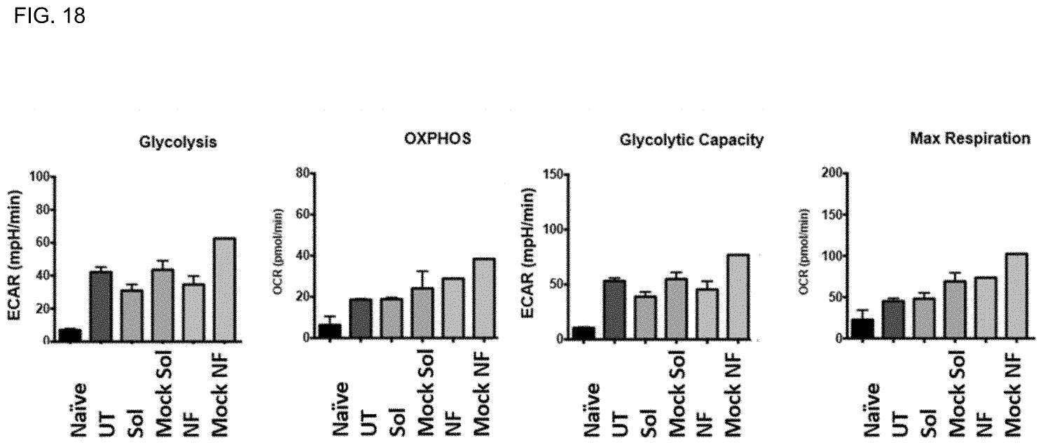

The Seahorse instrument measures oxygen consumption rate (OCR) and extracellular acidification rate (ECAR) as indicators of mitochondrial respiration and glycolysis respectively. An illustration of how Seahorse data is analysed is seen below (FIGS. 16A and 16B). The raw data traces from one donor either soluporated or nucleofected is shown in FIG. 17. The glycolysis, oxidative phosphorylation, glycolytic capacity and maximal respiration is shown in FIG. 18 as determined using the calculations in FIGS. 16A and 16B.

### Oxidative Phosphorylation (OCR Data)

Seahorse experimental set up is shown in FIGS. 16A and 16B. For OCR data looking at the UT, the OCR rate dips slightly when the oligo is added, rises upon FCCP (Carbonyl cyanide-p-trifluoromethoxyphenylhydrazone) stimulation, and falls again upon addition of Rot/AA. The modulators included in this assay kit are Oligomycin, Carbonyl cyanide-4 (trifluoromethoxy) phenylhydrazone (FCCP), Rotenone, and Antimycin A, which should stimulate the oxygen consumption rate patterns seen in FIGS. 16A and 16B. In the experiment shown, mock soluporation tracks practically identically to the UT showing that the soluporation process itself does not interfere with normal cellular oxidative phosphorylation (FIG. 17). Soluporation with mRNA-GFP shows a slight increase in the OCR rate following FCCP stimulation compared to the UT from approximately 60 pmol/min to close to 100 pmol/min. With nucleofected cells, however, the increase in OCR rate is more evident following FCCP stimulation with the OCR rate rising to almost 150 pmol/min (FIG. 17 and FIG. 18). Thus, the spare respiratory capacity (SRC) of nucleofected cells, in this experiment, is further from that of UT than soluporated cells SRC is. It is important to note that this is a snapshot of the metabolism of these cells at this time point 18 hours post transfection.

### Glycolytic Measurements (ECAR)

Seahorse experimental set up is as FIGS. 16A and 16B. For ECAR data looking at the UT, there is a minor increase in ECAR rate upon addition of oligo and then a dramatic dip following 2DG, mirroring the pattern seen in FIGS. 16A and 16B. Similar to the OCR rate, the mock soluporation ECAR rate tracks almost identically to the UT showing that the soluporation process itself does not interfere with normal cellular glycolysis (FIG. 17). Soluporation with mRNA-GFP shows a slight increase in the ECAR rate, compared to the UT both basally from approximately 50 mpH/min to close to 70 mpH/min and following oligo addition from approximately 60 mpH/min to close to 80 mpH/min. With nucleofected cells, however, the increase in OCR rate is more evident following FCCP stimulation with the OCR rate rising to almost 150 pmol/min (FIG. 17 and FIG. 18). The data suggests that there are no substantial or significant differences in cellular glycolysis (ECAR) in T cells subjected to the Solupore™ process compared to untreated or those subjected to nucleofection. This is true for both basal glycolytic measurements and measurements of the glycolytic capacity of the T cells. The glycolytic capacity is the max rate of glycolysis that the cell can achieve when forced to do so and is a measure of the glycolytic machinery available to the cell.

### Example 9: CAR Plus Data

The SOLUPORE™ delivery method was used in conjunction with cells that have undergone an additional cargo delivery manipulation method. For example, the SOLUPORE™ delivery method was used to delivery exogenous cargo, e.g., mRNA, to cells that had already been virally transduced. Alternatively, the SOLUPORE™ delivery method is used first to deliver exogenous cargo, e.g., mRNA, and then the cells are subjected to an additional delivery manipulation method, e.g., viral transduction.

The term “CAR plus” refers to a population of cells that have been either 1) virally transduced, and then followed by additional intracellular delivery method (e.g., the SOLUPORE™ delivery method, electroporation, or nucleofection, or any combination thereof), or 2) the SOLUPORE™ delivery method was used to deliver exogenous cargo, and then the cells are subjected to an additional intracellular delivery method (e.g., viral transduction, the SOLUPORE™ delivery method, electroporation, or nucleofection, or any combination thereof). Where cells have first been virally transduced, and then subjected to intracellular delivery using the SOLUPORE™ delivery method, viral components may still be present.

The feasibility of the SOLUPORE™ delivery method was assessed of virally transduced CAR T cells. GFP expression and viability in LV (lentiviral) CARP T cells (3 donors×n=1) was evaluated. FIGS. 19A and 19B, demonstrated the feasibility of the SOLUPORE™ delivery method for generating cells with multiple modifications. A 65% transfection efficiency (FIG. 19A) was observed using the SOLUPORE™ delivery method in virally-transduced CAR T cells (63%, 60%, and 67% GFP+ in CAR+ T cells across 3 donors), and a greater than 80% viability at 24 hours was also observed (FIG. 19B).

The following materials and methods were used in the studies described herein.

### Cell Isolation and Culture

PBMC were isolated from fresh leukopaks using lymphoprep density gradient medium (StemCell) and cryopreserved using standard methods. Upon thaw, PBMC were initiated (i.e., stimulated or activated) to T cells using antibodies specific for cell surface markers on T cells, e.g., soluble CD3 (clone: OKT3) and CD28 (clone: 15E8) antibodies (both Miltenyi Biotech), each at 100 ng/ml. Cells were initiated for 3 days in complete culture media consisting of CTS OpTimizer+supplement (Gibco) with 5% Physiologix serum replacement (Nucleus Biologics), 1% L-Glutamine and 250 IU/ml IL-2 (CellGenix). Human CD3+ T cells were isolated directly from leukopaks at 24 hours post-collection using a MultiMACS 24 (Miltenyi Biotech) and Straight from Leukopak CD4 and CD8 T cell reagents (Milteyni Biotech) according to manufacturer's instructions. T cells were cultured at a density of 1×10⁶/ml in CTS culture media (Gibco) supplemented with 2 mM L-glutamine and 250 Um′ IL-2 (CellGenix). Cells were activated with anti-CD3/CD28 coated beads (Cell Therapy Systems (CTS) Dynabeads) at a 2:1 bead to cell ratio.

### SOLUPORE™ Delivery Method

The SOLUPORE™ delivery method was adapted from that previously described. Cells were transferred to either 96-well filter bottom plates (Agilent) at 3.5×10⁵ cells per well or pods (Avectas) at 6×10⁶ cells per pod using the SOLUPORE™ delivery method. Culture medium was removed from the 96-well plates by centrifugation at 350×g for 120 sec and from the pods by gravity flow. Cargos were combined with delivery solution (32.5 mM sucrose, 106 mM potassium chloride, 5 mM (4-(2-hydroxyethyl)-1-piperazineethanesulfonic acid) (HEPES) in water) and 1 μl or 50 μl was delivered onto cells in 96-well plates and pods respectively. For delivery of mRNA, delivery solutions also contained 12% v/v ethanol. For delivery of ribonucleoproteins (RNPs), delivery solution also contained 25 mM ammonium acetate and 10% v/v ethanol. Following a 30 sec incubation at room temperature, 50-2000 μl 0.5X phosphate buffered saline solution (68.4 mM sodium chloride, 1.3 mM potassium chloride, 4.0 mM sodium hydrogen phosphate, 0.7 mM potassium dihydrogenphosphate) was added and after 30 sec, complete culture medium was added. The complete culture medium includes CTS OpTimizer+supplement (Gibco) with 5% Physiologix serum replacement (Nucleus Biologics), 1% L-Glutamine and 250 IU/ml IL-2 (CellGenix).

### Electroporation

Cells were electroporated using standard methods, the 4D-Nucleofector System (Lonza) (20 μl nucleocuvette or 100 μl nucleocuvette format) as per manufacturer's protocol and P3 Primary Cell 4-D Nucleofector Solution using the preloaded FI-115 and EO-115 pulse programs.

GFP mRNA and CAR mRNA Delivery

GFP mRNA (model cargo) and CD19 CAR mRNA (functional cargo) (both TriLink Biotechnologies) were delivered to a final concentration of 2 μg per million cells and 3.3 μg/1×10⁶ cells respectively for both the SOLUPORE™ delivery method and electroporation. CD19 CAR expression was evaluated using a biotin-conjugated CD19 CAR detection reagent (Miltenyi Biotec) followed by Steptavidin-PE with 7-Aminoactinomycin D (7AAD) as a viability stain.

CD19 CAR sequence
(SEQ ID NO: 8)
TGATATCCAGATGACCCAGACCACCAGCAGCCTGTCTGCCTCTCTGGGCGA
TAGAGTGACCATCAGCTGTAGAGCCAGCCAGGACATCAGCAAGTACCTGAA
CTGGTATCAGCAGAAACCCGACGGCACCGTGAAGCTGCTGATCTACCACAC
CAGCAGACTGCACAGCGGCGTGCCAAGCAGATTTTCTGGCAGCGGCTCTGG
CACCGACTACAGCCTGACAATCAGCAACCTGGAACAAGAGGATATCGCTAC
CTACTTCTGCCAGCAAGGCAACACCCTGCCTTACACCTTTGGCGGAGGCAC
CAAGCTGGAAATCACCGGCTCTACAAGCGGCAGCGGCAAACCTGGATCTGG
CGAGGGATCTACCAAGGGCGAAGTGAAACTGCAAGAGTCTGGCCCTGGACT
GGTGGCCCCATCTCAGTCTCTGAGCGTGACCTGTACAGTCAGCGGAGTGTC
CCTGCCTGATTACGGCGTGTCCTGGATCAGACAGCCTCCTCGGAAAGGCCT
GGAATGGCTGGGAGTGATCTGGGGCAGCGAGACAACCTACTACAACAGCGC
CCTGAAGTCCCGGCTGACCATCATCAAGGACAACTCCAAGAGCCAGGTGTT
CCTGAAGATGAACAGCCTGCAGACCGACGACACCGCCATCTACTATTGCGC
CAAGCACTACTACTACGGCGGCAGCTACGCCATGGATTATTGGGGCCAGGG
CACCAGCGTGACCGTGTCTAGTACAACAACCCCTGCTCCTCGGCCTCCTAC
ACCAGCTCCTACAATTGCCAGCCAGCCACTGTCTCTGAGGCCCGAAGCTTG
TAGACCTGCTGCTGGCGGAGCCGTGCATACAAGAGGACTGGATTTCGCCTG
CGACTTCTGGGTGCTCGTGGTTGTTGGCGGAGTGCTGGCCTGTTACAGCCT
GCTGGTTACCGTGGCCTTCATCATCTTTTGGGTCCGAAGCAAGCGGAGCCG
GCTGCTGCACTCCGACTACATGAACATGACCCCTAGACGGCCCGGACCTAC
CAGAAAGCACTACCAGCCTTACGCTCCTCCTAGAGACTTCGCCGCCTACAG
ATCCAAGCGGGGCAGAAAGAAACTGCTCTACATCTTCAAGCAGCCCTTCAT
GCGGCCCGTGCAGACCACACAAGAGGAAGATGGCTGCTCCTGCAGATTCCC
CGAGGAAGAAGAAGGCGGCTGCGAGCTGAGAGTGAAGTTCAGCAGATCCGC
CGACGCTCCCGCCTATAAGCAGGGACAGAACCAGCTGTACAACGAGCTGAA
CCTGGGGAGAAGAGAAGAGTACGACGTGCTGGACAAGCGGAGAGGCAGGGA
TCCTGAAATGGGCGGCAAGCCCAGACGGAAGAATCCTCAAGAGGGCCTGTA
TAATGAGCTGCAGAAAGACAAGATGGCCGAGGCCTACAGCGAGATCGGAAT
GAAGGGCGAGCGCAGAAGAGGCAAGGGACACGATGGACTGTACCAGGGACT
GAGCACCGCCACCAAGGATACCTATGACGCCCTGCACATGCAGGCCCTGCC
TCCAAGATAAGTCGACAATCAA

### RNP Complexes

Cas9 protein (Integrated DNA Technologies) was delivered at a final concentration of 3.3 μg/1×10⁶ cells for both the SOLUPORE™ delivery method, and electroporation, precomplexed with a 2 molar excess of guide RNA (gRNA) (CRISPR-associated endonuclease Cas9 (Cas9)—2.48 μM and gRNA 4.96 μM; Integrated DNA Technologies). The sequence for human TRAC (T-cell receptor alpha) targeting gRNA was AGAGTCTCTCAGCTGGTACA (SEQ ID NO: 25) and for human PDCD1 (Programmed cell death protein 1) targeting gRNA was GTCTGGGCGGTGCTACAACT (SEQ ID NO: 26). CD3 (cluster of differentiation 3) expression was analysed by flow cytometry on day 2 post-transfection. For PDCD1 gene INDEL (insertion or deletion of bases) analysis, cells were harvested on day 4 post-transfection.

### Flow Cytometry Analysis

Flow cytometry was performed using NovoCyte 3000. Data were examined using NovoExpress software (Acea Biosciences).

### PDCD1 Gene INDEL Analysis

Genomic DNA was extracted from cells using the MagNA Pure Compact Nucleic Acid Isolation Kit 1 (Roche). A PCR was performed to amplify a 305 bp region around the edit site (forward primer—AGCACTGCCTCTGTCACTCTCG (SEQ ID NO: 40); reverse primer—AGGGACTGAGGGTGGAAGGTC (SEQ ID NO: 12); Integrated DNA Technologies). The PCR product was sequenced by Sanger sequencing (Eurofins Genomics) and TIDE (Tracking of Indels by Decomposition) analysis was carried out on the sequence at TIDE (https://tide.nki.n1/).

### Cytokine Release Analysis

GFP mRNA was delivered to activated human T cells from 5 healthy donors using either the SOLUPORE™ delivery method or nucleofection. Four hours post-treatment, cells were reseeded into 96-well plates at 1×10⁶/ml and supernatants were collected each day for 5 days. Cell proliferation assays were carried out using a similar method where cells were counted and reseeded to 1×10⁶/ml daily. A custom Luminex Assay panel (Merck Millipore) was designed to measure 11 human analytes: IL-2 (interleukin 2), IFN-γ (interferon gamma), TNFα(tumor necrosis factor alpha), GM-CSF (Granulocyte-macrophage colony-stimulating factor), IL-8 (interleukin 8), IL-10 (interleukin 10), MIP-1α(macrophage inflammatory protein 1 alpha), MIP-1β (macrophage inflammatory protein 1 beta), Fractalkine, ITAC (Interferon—inducible T Cell Alpha Chemoattractant) and IL-17A (interleukin 17A). Supernatant samples were analysed in duplicate using the protocol for Human High Sensitivity T Cell Magnetic Bead Panel on a Luminex 200™ System (Merck Millipore).

### Gene Profiling

RNA was isolated from cells using the RNeasy Mini Kit (Qiagen) as per manufacturer's instructions. Transcripts were analysed using the NanoString nCounter Human CAR-T Characterization Panel (NanoString). Differential expression is displayed using log 2 fold change with tables filtered −1≥Log 2≤1. The NanoString panel is a comprehensive immune panel with 770 genes from 14 different immune cell types, common checkpoint inhibitors, CT (cancer/testis) antigens, and genes covering both the adaptive and innate immune response.

A table of the immune cell type gene coverage of the NanoString panel is provided below.

| Cell Type | Description |
|---|---|
| B Cells | B cells are the primary mediators of the |
| | humoral immune response, bearing antigen- |
| | specific B cell receptors and producing |
| | antibodies that can enable the immune |
| | system to respond to a broad variety of |
| | antigens. B cells can also function as |
| | MHC class II antigen presenting cells |
| | to stimulate T cell immunity. |
| T Cells | T-cells mediate cell-based immunity by |
| | recognizing primarily peptide antigens |
| | displayed on MHC class I or class II and |
| | either producing cytokines or directly |
| | killing the presenting cell. |
| TH1 | CD4+ T cell subset that produces IL2 |
| | and Interferon-gamma to promote cellular |
| | immunity by acting on CD8+ T Cells, NK |
| | Cells and Macrophages |
| Regulatory T | CD4+ T Cells that supress effector B and |
| cells (Treg) | T Cells and play a central role in |
| | suppresion of the immune response and |
| | tolerance to self-antigens |
| CD45 | CD45 is a common marker of all leukocytes, |
| | including B and T cells |
| CD8+ T Cells | A subset of T cells that are capable of |
| | binding cognate-antigen expressing cells |
| | via class I MHC and directly lysing them |
| | via perforin and granzymes. |
| Exhausted | T-cells overstimulated by antigen can |
| CD8+ T Cells | develop an “exhausted” phenotype, in which |
| | they are no longer effective in targeting |
| | antigen-bearing cells. |
| Cytotoxic Cells | These markers measure all cells capable |
| | of cytotoxic activity, which can include |
| | T, NKT, and NK-cells. |
| Dendritic Cells | Professional antigen presenting cells |
| | that internalize, process, and present |
| | antigens to lymphocytes via MHC class I |
| | and class II along with costimulatory |
| | signals to initiate cellular immune |
| | responses. |
| Macrophages | Pluripotent cells with critical roles in |
| | initiating innate and adaptive immune |
| | responses, phagocytosing abnormal cells, |
| | and regulating wound healing and tissue |
| | repair. |
| Mast Cells | Mast cells release histamine containing |
| | granules and other signals in order to |
| | promote inflammation and regulate allergic |
| | responses. |
| Neutrophils | Neutrophils are highly abundant cells that |
| | respond early to sites of infection or |
| | inflammation, phagocytose cellular debris, |
| | and promote downstream immunity. |
| Natural | Cytotoxic cells of the innate immune |
| Killer | system that are a significant source of |
| (NK) Cells | interferon-gamma and are capable of |
| | directly killing targeted cells via |
| | detection of a loss in MHC surface |
| | expression |
| NK CD56dim | The amount of CD56 present on an NK cell |
| cells | is indicative of its age and differentiation |
| | state; CD56 dim cells are mature NK cells, |
| | more commonly found in peripheral blood than |
| | secondary lymphoid tissues, and have the |
| | greatest cytolytic activity. |

### In Vivo Murine Engraftment Study

Human PBMC were transfected with 3 μM Alexa Fluor™-labelled 3 kDa dextran-Alexa488 using the SOLUPORE™ delivery method or nucleofection. On day 0, nonobese diabetic/severe combined immunodeficiency (NOD/SCID) IL-2Rγnull (NSG) mice were irradiated (2.4Gy). Four hours later, mice were injected intravenously with the PBMC (1×10\hat{ }6/g). For the course of the study (28 days) mice were observed carefully for signs of illness and specifically for the development of GvHD. On day 14, peripheral blood was harvested from the mice for analysis of human (CD45 (clone HI30, Biolegend), CD3 (clone UCHT1, Biolegend), CD4 (clone SK3, Biolegend), CD8 (clone SK1, Biolegend)) cell engraftment by flow cytometry. On sacrifice during the course of the study or at the end point of the study (day 28) spleens were harvested for analysis of human (CD45, CD3, CD4, CD8) cell engraftment by flow cytometry.

### In Vitro Cytotoxicity Assays

CD19 CAR mRNA was delivered to T cells using the SOLUPORE™ delivery method or electroporation. 24 h post transfection, cells were cryopreserved in CryoStor CS10 (Sigma Aldrich). In vitro cytotoxicity was measured with an impedance assay using the xCELLigence® Real-Time Cell Analyzer Single Plate (RTCA SP) instrument (ACEA Biosciences). Wells of the electronic microtiter plates were coated with 4 μg/ml CD40 (cluster of differentiation 40) (ACEA Biosciences) for 3 h. RAJI cells (ATCC©) were seeded at 5×10⁴ cells/well and allowed to adhere overnight. The following day, 19-21 h later, CAR T cells were thawed, counted and added to the RAJI cells at the following Effector: Target ratios: 2.5:1, 1.25:1, 0.6:1, 0.3:1, 0.15:1. Impedance was monitored every 1 min for 4 h, 5 min for 8 h and then 15 min for at least 92 h. Cell indexes (CIs) were normalized to CI of the time-point when CAR-T cells were added and specific lysis was calculated in compared with control effector cell-only cultures.

### In Vivo Murine CAR T Cell Efficacy Study

CD19 CAR mRNA was delivered to T cells using the SOLUPORE™ delivery method or nucleofection and cell were cryopreserved. NSG™ mice were engrafted on Day 0 with CD19+ RAJI-luciferase tumor cells (2.5×10⁵, intravenous) and mice were randomized across treatment groups based on body weight. On day 3, CAR T cell were thawed and 1×10⁶, 2×10⁶ or 4×10⁶ cells were injection per animal. On day 15, bioluminescence imaging was carried out and animals were euthanized by CO₂ asphyxiation.

### Statistics

An unpaired Student t test was used to assess the significance of comparative engraftment of tumor or CAR T cells in vivo. 95% confidence interval was used to compare the mean average of each duplicate analysed on the Luminex. A two-way ANOVA (analysis of variance) was used to compare the mean of each group with the untreated control with at each timepoint, **P<0.01; *P<0.05. All statistical analysis was performed using GraphPad Prism 8.0.

### Cell Phenotype Analysis and Metabolism Assays

The surface expression of T cell activation markers and the glycolytic activity of T cells generated by Avectas where assessed using Flow Cytometry and Seahorse Analysis respectively. Briefly T cells were activated using dyna beads and IL-2 for 19 hours after which cells were either untreated (UT), mock transfected using Soluporation (Sol Mock) or Nucleofection (NF Mock) or transfected with GFP mRNA using Soluporation (Sol) or Nucleofection (NF). Cells were analyzed using a panel of mAb specific for T cell related activation/exhausted surface markers (PD-1 and CD69 being the primary targets). During analysis, GFP+ cells were gated for Soluporation and Nucleofection and compared to Untreated activated cells (UT). For extracellular flux analysis 2×10\hat{ }5 T cells were plated in quadruplicates onto Seahorse culture plates and rested overnight in IL-2 media. The following day cells were adhered to Seahorse culture plate using CellTak and re-suspended in Seahorse culture media (controlled for pH and nutrient content). Cells where then analyzed using the Seahorse analyzer with 4 measurements obtained for each time point. In addition to Extracellular Acidification Rate (ECAR), oxygen consumption (OCR) was also measured and represents rates of Oxidative Phosphorylation (OxPhos). Resting T cells utilize OxPhos but after activation “switch” to glycolytic metabolism.

### Lactate Assay

The L-lactate production of activated T cells transfected with mRNA-GFP using soluporation or Nucleofection (program EO115) was analyzed using the ChromaDazzle Lactate Assay Kit (AssayGenie). 6 hours post transfection supernatants were harvested and stored at −20° C.

TABLE 7
Dataset S1 Study at 6 hours (Electroporation FI-115 at 6 hours)
Electroporation FI-115 at 6 hr

| Gene name | | Gene name | |
|---|---|---|---|
| Ordered | | Ordered by | |
| alphabetically | | level of change | |
| ACAD10 | −1.27 | FOSB | 6.15 |
| ACSF2 | −3.77 | CD200 | 5.51 |
| ACSL5 | −1.03 | SEC7A5 | 3.75 |
| ACTN1 | −2.7 | IL2 | 3.64 |
| AKT1 | −1.09 | IRF4 | 3.5 |
| ALDH3A2 | −1.83 | FOS | 3.05 |
| ALDOC | −1.97 | CXCL8 | 2.97 |
| ATP5PD | −1.14 | IFNG | 2.94 |
| BATF3 | 2.72 | SIK1 | 2.76 |
| BATF | 1.22 | IFIT3 | 2.74 |
| BID | −1.03 | BATF3 | 2.72 |
| BUB1 | −1.07 | CD40LG | 2.57 |
| CASP8 | −1.03 | CCL3/L1 | 2.53 |
| CBR4 | −1.11 | CD69 | 2.29 |
| CCL3/L1 | 2.53 | GLUD1/2 | 2.24 |
| CCL4/L1 | 1.84 | EGR1 | 2.19 |
| CCR2 | −2.57 | FASLG | 2.18 |
| CCR5 | −3.14 | HK2 | 2.12 |
| CCR6 | −1.38 | CXCL10 | 2.06 |
| CCR7 | −1.7 | NFKBIA | 2 |
| CD160 | 1.06 | IL21R | 1.96 |
| CD200 | 5.51 | CCL4/L1 | 1.84 |
| CD40LG | 2.57 | MTHFD2 | 1.76 |
| CD4 | −1.81 | TBX21 | 1.75 |
| CD68 | −1.09 | PTGER4 | 1.74 |
| CD69 | 2.29 | NFIL3 | 1.71 |
| CD80 | 1.15 | NFATC1 | 1.66 |
| CD8A | −1.74 | JUN | 1.56 |
| CD8B | −1.79 | STAT5A | 1.5 |
| CD96 | −1.63 | NFKB2 | 1.47 |
| CISH | −2.41 | NBL1 | 1.33 |
| CLCF1 | −1.69 | XCL1/2 | 1.33 |
| CMIP | −1.11 | CTLA4 | 1.31 |
| COX5B | −1.13 | SLC3A2 | 1.28 |
| CTLA4 | 1.31 | BATF | 1.22 |
| CTSD | −1.54 | HIF1A | 1.18 |
| CTSW | −2 | TFRC | 1.17 |
| CX3CR1 | −1.98 | CD80 | 1.15 |
| CXCL10 | 2.06 | MTHFD1L | 1.15 |
| CXCL8 | 2.97 | PPT2 | 1.15 |
| CXCR3 | −1.01 | ICOS | 1.14 |
| CXCR6 | −2.48 | IL12RB2 | 1.13 |
| CYBB | −1.89 | VSIR | 1.13 |
| DECR1 | −1.31 | GARS | 1.11 |
| DGLUCY | −1.25 | PGAM1 | 1.11 |
| DHRS4 | −1.38 | GLS | 1.1 |
| DLL1 | −1.22 | DUSP1 | 1.09 |
| DOCK2 | −1.22 | CD160 | 1.06 |
| DUSP1 | 1.09 | FYN | 1.06 |
| EGR1 | 2.19 | STAT3 | 1.05 |
| FASLG | 2.18 | IL36A | 1.01 |
| FCGR3A/B | −2.03 | CXCR3 | −1.01 |
| FOSB | 6.15 | ISG15 | −1.01 |
| FOS | 3.05 | TGFBR1 | −1.02 |
| FOXP3 | −1.84 | TRGV4 | −1.02 |
| FYN | 1.06 | ACSL5 | −1.03 |
| GARS | 1.11 | BID | −1.03 |
| GLS2 | −1.85 | CASP8 | −1.03 |
| GLS | 1.1 | PPP2R5D | −1.03 |
| GLUD1/2 | 2.24 | GNG10 | −1.05 |
| GNAI2 | −1.9 | TRBV7-8 | −1.05 |
| GNG10 | −1.05 | IKZF4 | −1.06 |
| GPI | −1.15 | BUB1 | −1.07 |
| GRK2 | −2.01 | TRBV29-1 | −1.07 |
| GZMA | −2.6 | MAP2K2 | −1.08 |
| GZMH | −1.64 | AKT1 | −1.09 |
| GZMK | −2.11 | CD68 | −1.09 |
| GZMM | −1.81 | IL18BP | −1.09 |
| HACD4 | −1.54 | RPTOR | −1.09 |
| HAVCR2 | −1.28 | TRAV41 | −1.1 |
| HDAC7 | −1.25 | CBR4 | −1.11 |
| HIF1A | 1.18 | CMIP | −1.11 |
| HK2 | 2.12 | IFI35 | −1.11 |
| HLA-DRA | −1.17 | KLRD1 | −1.11 |
| ICOS | 1.14 | NEDD8 | −1.11 |
| IFI30 | −3.6 | PARP1 | −1.11 |
| IFI35 | −1.11 | TRAV8-3 | −1.12 |
| IFI6 | −1.34 | COX5B | −1.13 |
| IFIT3 | 2.74 | ATP5PD | −1.14 |
| IFITM3 | −1.42 | SRR | −1.14 |
| IFNG | 2.94 | GPI | −1.15 |
| IKBKE | −1.56 | SELL | −1.15 |
| IKZF4 | −1.06 | TRGC1 | −1.15 |
| IL10RA | −1.7 | HLA-DRA | −1.17 |
| IL12RB1 | −1.93 | MTHFR | −1.17 |
| IL12RB2 | 1.13 | SMAD3 | −1.17 |
| IL16 | −2.91 | PCCA | −1.2 |
| IL18BP | −1.09 | TRAV10 | −1.21 |
| IL21R | 1.96 | DLL1 | −1.22 |
| IL2 | 3.64 | DOCK2 | −1.22 |
| IL32 | −3.16 | TRAV25 | −1.22 |
| IL36A | 1.01 | TRAV14 | −1.23 |
| IL7R | −1.39 | SLC2A11 | −1.24 |
| IRF3 | −1.48 | DGLUCY | −1.25 |
| IRF4 | 3.5 | HDAC7 | −1.25 |
| IRF5 | −1.54 | TRAV39 | −1.26 |
| ISG15 | −1.01 | ACAD10 | −1.27 |
| ITGB2 | −1.98 | TRAV26-2 | −1.27 |
| JUN | 1.56 | HAVCR2 | −1.28 |
| KIR3DL1/2 | −2.15 | RAI1 | −1.28 |
| KLRD1 | −1.11 | TRGV8 | −1.29 |
| KYAT1 | −1.93 | DECR1 | −1.31 |
| LAIR1 | −2.31 | TRBV28 | −1.31 |
| LAT | −2.16 | NPRL3 | −1.32 |
| LCK | −1.93 | SH2D1A | −1.32 |
| LTB | −2.17 | TRAV12-1 | −1.33 |
| MAGED1 | −1.39 | IFI6 | −1.34 |
| MAP2K2 | −1.08 | TRAC | −1.34 |
| MAP2K7 | −1.47 | TRAV18 | −1.35 |
| MAPK3 | −1.89 | RNASEL | −1.36 |
| MR1 | −2.08 | TRAV17 | −1.36 |
| MS4A1 | −1.95 | TRAV26-1 | −1.36 |
| MTHFD1L | 1.15 | TCF7 | −1.37 |
| MTHFD1 | −1.78 | TRAV12-2 | −1.37 |
| MTHFD2 | 1.76 | CCR6 | −1.38 |
| MTHFR | −1.17 | DHRS4 | −1.38 |
| NBL1 | 1.33 | IL7R | −1.39 |
| NCAPD2 | −1.62 | MAGED1 | −1.39 |
| NCAPG2 | −1.39 | NCAPG2 | −1.39 |
| NCR3 | −2.66 | TRAV27 | −1.39 |
| NEDD8 | −1.11 | TRDV1 | −1.39 |
| NFATC1 | 1.66 | IFITM3 | −1.42 |
| NFIL3 | 1.71 | TRAV38-1 | −1.42 |
| NFKB2 | 1.47 | PRKCB | −1.43 |
| NFKBIA | 2 | TRAV22 | −1.44 |
| NKG7 | −1.68 | TRAV24 | −1.44 |
| NOTCH1 | −1.5 | TRAV6 | −1.44 |
| NPRL3 | −1.32 | TRBV6-2 | −1.45 |
| OAS1 | −1.58 | MAP2K7 | −1.47 |
| OAS2 | −1.71 | IRF3 | −1.48 |
| OMA1 | −1.66 | TRBV14 | −1.49 |
| PARP1 | −1.11 | NOTCH1 | −1.5 |
| PCCA | −1.2 | STK11 | −1.5 |
| PECAM1 | −2.34 | TRAV1-2 | −1.5 |
| PFKL | −1.64 | TRAV4 | −1.5 |
| PGAM1 | 1.11 | TRBV2 | −1.5 |
| PIK3R2 | −1.86 | TRBV5-6 | −1.5 |
| PLCB2 | −1.56 | TRBV7-6 | −1.5 |
| PLCG1 | −1.69 | TRAT1 | −1.51 |
| PPP2R5D | −1.03 | TRAV8-6 | −1.51 |
| PPT2 | 1.15 | TRBV10-2 | −1.51 |
| PRF1 | −2.04 | PSMB10 | −1.52 |
| PRICKLE3 | −2.04 | TRAV3 | −1.53 |
| PRKCB | −1.43 | TRAV8-1 | −1.53 |
| PSMB10 | −1.52 | CTSD | −1.54 |
| PTGDR2 | −4.72 | HACD4 | −1.54 |
| PTGER4 | 1.74 | IRF5 | −1.54 |
| RAC2 | −2.03 | SMC2 | −1.54 |
| RAI1 | −1.28 | TRAV9-2 | −1.54 |
| RNASEL | −1.36 | TRBV4-3 | −1.54 |
| RPTOR | −1.09 | IKBKE | −1.56 |
| SCD | −1.56 | PLCB2 | −1.56 |
| SELL | −1.15 | SCD | −1.56 |
| SELPLG | −2.09 | TRAV34 | −1.56 |
| SGO2 | −3.16 | OAS1 | −1.58 |
| SH2D1A | −1.32 | TRAV29 | −1.58 |
| SH3BP2 | −1.79 | TRAV5 | −1.59 |
| SHMT1 | −2.15 | NCAPD2 | −1.62 |
| SIK1 | 2.76 | TRAV35 | −1.62 |
| SLC25A20 | −1.82 | CD96 | −1.63 |
| SLC27A3 | −3.02 | TRBC1/2 | −1.63 |
| SLC2A11 | −1.24 | GZMH | −1.64 |
| SLC3A2 | 1.28 | PFKL | −1.64 |
| SLC7A5 | 3.75 | TRAV12-3 | −1.64 |
| SMAD3 | −1.17 | TRAV23 | −1.64 |
| SMC2 | −1.54 | TRAV13-2 | −1.65 |
| SRR | −1.14 | OMA1 | −1.66 |
| STAT3 | 1.05 | TFDP1 | −1.67 |
| STAT5A | 1.5 | TRBV6-5 | −1.67 |
| STK11 | −1.5 | NKG7 | −1.68 |
| TBX21 | 1.75 | CLCF1 | −1.69 |
| TCF7 | −1.37 | PLCG1 | −1.69 |
| TFDP1 | −1.67 | CCR7 | −1.7 |
| TFRC | 1.17 | IL10RA | −1.7 |
| TGFBR1 | −1.02 | TRBV5-5 | −1.7 |
| TIMP1 | −2.49 | OAS2 | −1.71 |
| TNFSF13B | −2.1 | TRBV13 | −1.71 |
| TRAC | −1.34 | TRBV18 | −1.72 |
| TRAT1 | −1.51 | TRBV7-3 | −1.73 |
| TRAV10 | −1.21 | CD8A | −1.74 |
| TRAV1-1 | −2.51 | TRBV5-1 | −1.74 |
| TRAV12-1 | −1.33 | WAS | −1.74 |
| TRAV12-2 | −1.37 | TRAV20 | −1.75 |
| TRAV12-3 | −1.64 | TRAV21 | −1.77 |
| TRAV1-2 | −1.5 | MTHFD1 | −1.78 |
| TRAV13-2 | −1.65 | TRBV10-3 | −1.78 |
| TRAV14 | −1.23 | TRBV6-9 | −1.78 |
| TRAV17 | −1.36 | CD8B | −1.79 |
| TRAV18 | −1.35 | SH3BP2 | −1.79 |
| TRAV20 | −1.75 | TRAV30 | −1.79 |
| TRAV21 | −1.77 | CD4 | −1.81 |
| TRAV22 | −1.44 | GZMM | −1.81 |
| TRAV23 | −1.64 | USP18 | −1.81 |
| TRAV24 | −1.44 | SLC25A20 | −1.82 |
| TRAV25 | −1.22 | TRAV38-2 | −1.82 |
| TRAV26-1 | −1.36 | ALDH3A2 | −1.83 |
| TRAV26-2 | −1.27 | TRBV15 | −1.83 |
| TRAV27 | −1.39 | FOXP3 | −1.84 |
| TRAV29 | −1.58 | TRBV11-2 | −1.84 |
| TRAV30 | −1.79 | TRBV30 | −1.84 |
| TRAV34 | −1.56 | GLS2 | −1.85 |
| TRAV35 | −1.62 | PIK3R2 | −1.86 |
| TRAV38-1 | −1.42 | TRBV7-2 | −1.87 |
| TRAV38-2 | −1.82 | CYBB | −1.89 |
| TRAV39 | −1.26 | MAPK3 | −1.89 |
| TRAV3 | −1.53 | TRDC | −1.89 |
| TRAV41 | −1.1 | GNAI2 | −1.9 |
| TRAV4 | −1.5 | TRBV4-2 | −1.9 |
| TRAV5 | −1.59 | IL12RB1 | −1.93 |
| TRAV6 | −1.44 | KYAT1 | −1.93 |
| TRAV8-1 | −1.53 | LCK | −1.93 |
| TRAV8-3 | −1.12 | MS4A1 | −1.95 |
| TRAV8-6 | −1.51 | TRBV7-9 | −1.96 |
| TRAV9-2 | −1.54 | ALDOC | −1.97 |
| TRBC1/2 | −1.63 | TYROBP | −1.97 |
| TRBV10-2 | −1.51 | VAV1 | −1.97 |
| TRBV10-3 | −1.78 | CX3CR1 | −1.98 |
| TRBV11-1 | −2.19 | ITGB2 | −1.98 |
| TRBV11-2 | −1.84 | CTSW | −2 |
| TRBV12-3 | −2.2 | TRBV6-6 | −2 |
| TRBV12-5 | −2.14 | GRK2 | −2.01 |
| TRBV13 | −1.71 | FCGR3A/B | −2.03 |
| TRBV14 | −1.49 | RAC2 | −2.03 |
| TRBV15 | −1.83 | TRBV19 | −2.03 |
| TRBV18 | −1.72 | PRF1 | −2.04 |
| TRBV19 | −2.03 | PRICKLE3 | −2.04 |
| TRBV20-1 | −2.04 | TRBV20-1 | −2.04 |
| TRBV25-1 | −2.13 | MR1 | −2.08 |
| TRBV27 | −2.23 | SELPLG | −2.09 |
| TRBV28 | −1.31 | TNFSF13B | −2.1 |
| TRBV29-1 | −1.07 | GZMK | −2.11 |
| TRBV2 | −1.5 | TRBV25-1 | −2.13 |
| TRBV30 | −1.84 | TRBV12-5 | −2.14 |
| TRBV3-1 | −2.15 | TRIM34 | −2.14 |
| TRBV4-1 | −2.75 | KIR3DL1/2 | −2.15 |
| TRBV4-2 | −1.9 | SHMT1 | −2.15 |
| TRBV4-3 | −1.54 | TRBV3-1 | −2.15 |
| TRBV5-1 | −1.74 | TRBV5-4 | −2.15 |
| TRBV5-4 | −2.15 | LAT | −2.16 |
| TRBV5-5 | −1.7 | LTB | −2.17 |
| TRBV5-6 | −1.5 | TRBV11-1 | −2.19 |
| TRBV6-1 | −3.38 | TRBV9 | −2.19 |
| TRBV6-2 | −1.45 | TRBV12-3 | −2.2 |
| TRBV6-4 | −2.29 | TRBV27 | −2.23 |
| TRBV6-5 | −1.67 | TRBV6-4 | −2.29 |
| TRBV6-6 | −2 | LAIR1 | −2.31 |
| TRBV6-9 | −1.78 | PECAM1 | −2.34 |
| TRBV7-2 | −1.87 | CISH | −2.41 |
| TRBV7-3 | −1.73 | CXCR6 | −2.48 |
| TRBV7-6 | −1.5 | TIMP1 | −2.49 |
| TRBV7-8 | −1.05 | TRAV1-1 | −2.51 |
| TRBV7-9 | −1.96 | CCR2 | −2.57 |
| TRBV9 | −2.19 | GZMA | −2.6 |
| TRDC | −1.89 | NCR3 | −2.66 |
| TRDV1 | −1.39 | ACTN1 | −2.7 |
| TRGC1 | −1.15 | TRBV4-1 | −2.75 |
| TRGV4 | −1.02 | IL16 | −2.91 |
| TRGV8 | −1.29 | SLC27A3 | −3.02 |
| TRIM34 | −2.14 | CCR5 | −3.14 |
| TYROBP | −1.97 | IL32 | −3.16 |
| USP18 | −1.81 | SGO2 | −3.16 |
| VAV1 | −1.97 | TRBV6-1 | −3.38 |
| VSIR | 1.13 | IFI30 | −3.6 |
| WAS | −1.74 | ACSF2 | −3.77 |
| XCL1/2 | 1.33 | PTGDR2 | −4.72 |

Filter = >1 and <−1 log2 fold change (2 fold linear change)

TABLE 8
Dataset S1 Study at 6 hours (SOLUPORE ™ delivery method at 6 hours)
SOLUPORE ™ delivery method at 6 hr

| Gene name | | Gene name | |
|---|---|---|---|
| Ordered alphabetically | | Ordered by level of change | |
| CCL20 | −1.13 | FOSB | 2.21 |
| CD160 | −2.09 | IFIT3 | 1.88 |
| FOSB | 2.21 | JUN | −1.08 |
| IFIT3 | 1.88 | PSAT1 | −1.1 |
| IL2 | −1.15 | CCL20 | −1.13 |
| JUN | −1.08 | SGO2 | −1.13 |
| PSATI | −1.1 | IL2 | −1.15 |
| SGO2 | −1.13 | TRBV30 | −1.27 |
| TRAV1-1 | −1.38 | TRAV1-1 | −1.38 |
| TRBV30 | −1.27 | CD160 | −2.09 |

TABLE 9
Dataset S2 Study 1 at 24 hours Electroporation FI-115 at 24 hours
Electroporation FI-115 at 24 hr

| Gene name | | Gene name | |
|---|---|---|---|
| Ordered alphabetically | | Ordered by level of change | |
| AHR | 1.38 | CCL22 | 4.21 |
| BATF3 | 2.93 | FOSB | 3.23 |
| BATF | 1.38 | BATF3 | 2.93 |
| CCL22 | 4.21 | CD38 | 2.93 |
| CCL4/L1 | −1.71 | IDO1 | 2.46 |
| CCR8 | −2.16 | CXCL9 | 2.42 |
| CD19 | 1.57 | IRF4 | 2.37 |
| CD200 | 1.93 | JUN | 2.34 |
| CD244 | −1.31 | IRF8 | 2.28 |
| CD38 | 2.93 | IL26 | 2.19 |
| CD68 | 1.09 | CD200 | 1.93 |
| CTSW | −1.87 | IFIT3 | 1.92 |
| CX3CR1 | −2.68 | FOS | 1.84 |
| CXCL10 | 1.16 | IL12RB2 | 1.83 |
| CXCL9 | 2.42 | CD19 | 1.57 |
| EOMES | −2.33 | MT2A | 1.39 |
| FASLG | −1.41 | AHR | 1.38 |
| FCGR3A/B | −2.54 | BATF | 1.38 |
| FOSB | 3.23 | SLC7A5 | 1.26 |
| FOS | 1.84 | ICOSLG | 1.21 |
| GZMA | −1.66 | CXCL10 | 1.16 |
| GZMH | −1.8 | LTA | 1.15 |
| GZMK | −1.68 | IL2 | 1.11 |
| ICOSLG | 1.21 | CD68 | 1.09 |
| IDO1 | 2.46 | NFKBIA | 1.09 |
| IFIT1 | −1.57 | SLC3A2 | 1.06 |
| IFIT3 | 1.92 | MTHFD1L | 1.02 |
| IL12RB2 | 1.83 | RPTOR | 1.02 |
| IL26 | 2.19 | NFIL3 | 1.01 |
| IL2 | 1.11 | ZBTB16 | −1.04 |
| IL7R | −1.34 | KLRK1 | −1.05 |
| IRF4 | 2.37 | RORC | −1.07 |
| IRF8 | 2.28 | KLRG1 | −1.09 |
| ITGAM | −1.86 | TRBV16 | −1.09 |
| JUN | 2.34 | XCL1/2 | −1.09 |
| KIR3DL1/2 | −1.25 | PIK3R3 | −1.1 |
| KLRB1 | −1.55 | TRGC1 | −1.1 |
| KLRG1 | −1.09 | PECAM1 | −1.12 |
| KLRK1 | −1.05 | SELL | −1.19 |
| LAIR1 | −1.66 | KIR3DL1/2 | −1.25 |
| LTA | 1.15 | TRGC2 | −1.27 |
| MT2A | 1.39 | CD244 | −1.31 |
| MTHFD1L | 1.02 | NCAM1 | −1.33 |
| NCAM1 | −1.33 | IL7R | −1.34 |
| NCR1 | −1.72 | TYROBP | −1.39 |
| NFIL3 | 1.01 | FASLG | −1.41 |
| NFKBIA | 1.09 | NT5E | −1.43 |
| NT5E | −1.43 | TIMP1 | −1.43 |
| PECAM1 | −1.12 | TRGV2 | −1.45 |
| PIK3R3 | −1.1 | PRF1 | −1.46 |
| PPARD | −2.16 | KLRB1 | −1.55 |
| PRF1 | −1.46 | IFIT1 | −1.57 |
| RORC | −1.07 | TRGV8 | −1.65 |
| RPTOR | 1.02 | GZMA | −1.66 |
| SELL | −1.19 | LAIR1 | −1.66 |
| SLC3A2 | 1.06 | GZMK | −1.68 |
| SLC7A5 | 1.26 | CCL4/L1 | −1.71 |
| TIMP1 | −1.43 | NCR1 | −1.72 |
| TRBV16 | −1.09 | GZMH | −1.8 |
| TRGC1 | −1.1 | ITGAM | −1.86 |
| TRGC2 | −1.27 | CTSW | −1.87 |
| TRGV2 | −1.45 | CCR8 | −2.16 |
| TRGV8 | −1.65 | PPARD | −2.16 |
| TYROBP | −1.39 | EOMES | −2.33 |
| XCL1/2 | −1.09 | FCGR3A/B | −2.54 |
| ZBTB16 | −1.04 | CX3CR1 | −2.68 |

Filter = >1 and <−1 log2 fold change (2 fold linear change)
* No genes were identified in the group using the SOLUPORE ™ delivery method group at 24 hours

TABLE 10
Dataset S3 Study 2 at 24 hours electroporation FI-115 at 24 hours
Electroporation FI-115 at 24 hr

| Gene name | | Gene name | |
|---|---|---|---|
| Ordered alphabetically | | Ordered by level of change | |
| AHR | 1.29 | CD200 | 2.92 |
| BATF | 1.23 | FOSB | 2.82 |
| BATF3 | 2.05 | TNFRSF11A | 2.44 |
| CCL22 | 2.09 | CD38 | 2.33 |
| CCL4/L1 | −1.42 | CCL22 | 2.09 |
| CD19 | 1.58 | BATF3 | 2.05 |
| CD200 | 2.92 | IFIT3 | 1.99 |
| CD244 | −2.26 | JUN | 1.96 |
| CD38 | 2.33 | IRF8 | 1.94 |
| CD68 | 1.85 | CD68 | 1.85 |
| CTSW | −1.64 | FOS | 1.82 |
| CX3CR1 | −2.12 | IRF4 | 1.81 |
| FCGR3A/B | −1.4 | CD19 | 1.58 |
| FOS | 1.82 | IL12RB2 | 1.46 |
| FOSB | 2.82 | STAT1 | 1.44 |
| GZMA | −1.36 | AHR | 1.29 |
| GZMH | −1.08 | ICOSLG | 1.27 |
| GZMK | −1.54 | BATF | 1.23 |
| ICOSLG | 1.27 | MT2A | 1.14 |
| IFIT3 | 1.99 | PLCB3 | 1.14 |
| IL12RB2 | 1.46 | NFIL3 | 1.11 |
| IL23R | −1.95 | TRGC2 | −1.03 |
| IL7R | −1.04 | IL7R | −1.04 |
| IRF4 | 1.81 | SELL | −1.05 |
| IRF8 | 1.94 | GZMH | −1.08 |
| ITGAM | −1.64 | TRGV2 | −1.12 |
| JUN | 1.96 | PRF1 | −1.14 |
| KLRB1 | −1.23 | LAIR1 | −1.2 |
| LAIR1 | −1.2 | NT5E | −1.22 |
| MT2A | 1.14 | TCL1A | −1.22 |
| NCR1 | −1.28 | KLRB1 | −1.23 |
| NFIL3 | 1.11 | NCR1 | −1.28 |
| NT5E | −1.22 | GZMA | −1.36 |
| PLCB3 | 1.14 | FCGR3A/B | −1.4 |
| PRF1 | −1.14 | TYROBP | −1.4 |
| SELL | −1.05 | CCL4/L1 | −1.42 |
| STAT1 | 1.44 | TIMP1 | −1.42 |
| TCL1A | −1.22 | GZMK | −1.54 |
| TIMP1 | −1.42 | CTSW | −1.64 |
| TNFRSF11A | 2.44 | ITGAM | −1.64 |
| TRGC2 | −1.03 | IL23R | −1.95 |
| TRGV2 | −1.12 | CX3CR1 | −2.12 |
| TYROBP | −1.4 | CD244 | −2.26 |

Filter = >1 and <−1 log2 fold change (2 fold linear change)

TABLE 11
Dataset S3 Study 2 at 24 hours using SOLUPORE ™ delivery method
SOLUPORE ™ delivery method at 24 hr

| Gene name | | Gene name | |
|---|---|---|---|
| Ordered alphabetically | | Ordered by level of change | |
| BATF3 | 1.08 | IFIT1 | 2.06 |
| FOSB | 1.14 | IFIT3 | 1.77 |
| IFIT1 | 2.06 | OAS1 | 1.19 |
| IFIT2 | 1.08 | USP18 | 1.17 |
| IFIT3 | 1.77 | FOSB | 1.14 |
| MX1 | 1.02 | BATF3 | 1.08 |
| OAS1 | 1.19 | IFIT2 | 1.08 |
| TNFRSF11A | 1.08 | TNFRSF11A | 1.08 |
| USP18 | 1.17 | MX1 | 1.02 |

Filter = >1 and <−1 log2 fold change (2 fold linear change)

TABLE 12
Dataset S3 Study 2 at 24 hours comparison between
FI-115 electroporation and EO-115 electroporation

| Electroporation EO-115 | | FI-115 comparison |
|---|---|---|
| BATF3 | 1.45 | 2.05 |
| CCL22 | 1.46 | 2.09 |
| CCL3/L1 | −1.11 | −0.5 |
| CCR8 | −1.29 | −0.865 |
| CD19 | 1.23 | 1.58 |
| CD200 | 2.24 | 2.92 |
| CD38 | 1.06 | 2.33 |
| CD68 | 1.55 | 1.85 |
| CD9 | 1.03 | 0.996 |
| FOS | 1.6 | 1.82 |
| FOSB | 2.74 | 2.82 |
| ICOSLG | 1.03 | 1.27 |
| IFIT1 | −1.07 | −0.868 |
| JUN | 1.64 | 1.96 |
| MT2A | 1.01 | 1.14 |
| TNFRSF11A | 1.66 | 2.44 |

Filter = >1 and <−1 log2 fold change (2 fold linear change)

TABLE 13
T cells 6 hr post- Delivery (using SOLUPORE ™ delivery method)

| Genes with less | Genes with greater | Genes with greater |
|---|---|---|
| than 2 fold change | than 2 fold change | than 5 fold change |
| BATF3 | FOSB | None |
| GLUD1/2 | IFIT3 | |
| NFKB2 | JUN | |
| CD99 | PSAT1 | |
| CD40 | CCL20 | |
| AFDN | SGO2 | |
| AHR | IL2 | |
| CD9 | TRBV30 | |
| CLCF1 | TRAV1-1 | |
| CDKN1A | CD160 | |
| CTNNA1 | | |
| PRKCD | | |
| VAV3 | | |
| PTGDR2 | | |
| CCL3/L1 | | |
| EOMES | | |
| CD80 | | |
| MAF | | |
| ICOSLG | | |
| FAS | | |
| ICAM1 | | |
| SOS2 | | |
| ACADVL | | |
| IL36A | | |
| PKM | | |
| IL2RA | | |
| TICAM1 | | |
| CXCL8 | | |
| CD7 | | |
| NFKBIA | | |
| PPARA | | |
| SMAD5 | | |
| CXCR4 | | |
| TGFB1 | | |
| MTCP1 | | |
| SMURF1 | | |
| PPP3CA | | |
| RUNX3 | | |
| FLCN | | |
| KLRD1 | | |
| PIK3R2 | | |
| ACVR1C | | |
| CS | | |
| CX3CR1 | | |
| NOTCH2 | | |
| STAT6 | | |
| RELA | | |
| TNFRSF18 | | |
| IFIT2 | | |
| TNFRSF4 | | |
| CCR5 | | |
| TRIM26 | | |
| CD68 | | |
| DVL2 | | |
| IFI30 | | |
| IRF7 | | |
| STAT3 | | |
| BCL6 | | |
| GATA3 | | |
| APC | | |
| CD6 | | |
| LAG3 | | |
| IL12RB2 | | |
| IKZF4 | | |
| BATF | | |
| RORA | | |
| SLC7A5 | | |
| FNIP1 | | |
| NFIL3 | | |
| CCR2 | | |
| CASP8 | | |
| IFNGR2 | | |
| CD45R0 | | |
| GZMB | | |
| NCAPH | | |
| SMAD3 | | |
| SOCS4 | | |
| NOTCH1 | | |
| BTBD6 | | |
| OAS3 | | |
| IL15 | | |
| FOXP3 | | |
| OASL | | |
| NR3C1 | | |
| CD45RA | | |
| TGIF2 | | |
| AKT1 | | |
| IRF1 | | |
| PRKAB2 | | |
| OAS1 | | |
| RARG | | |
| ADD1 | | |
| IDO1 | | |
| PPT2 | | |
| IL23A | | |
| UBE2I | | |
| ACACA | | |
| SGK3 | | |
| CDC42 | | |
| SELPLG | | |
| IL4R | | |
| NFATC1 | | |
| PPP2R5D | | |
| CD27 | | |
| NSD2 | | |
| OAT | | |
| TBX21 | | |
| HIF1A | | |
| TOLLIP | | |
| MPC2 | | |
| IL2RG | | |
| BCL2L1 | | |
| TYK2 | | |
| IFNAR1 | | |
| MAP2K2 | | |
| WDR45 | | |
| HADHB | | |
| TRAF3 | | |
| MT2A | | |
| LAMP1 | | |
| PRF1 | | |
| ATG14 | | |
| IL10RA | | |
| NCR1 | | |
| CD8B | | |
| CTNNB1 | | |
| DLL1 | | |
| CKAP5 | | |
| MTHFD2 | | |
| FOS | | |
| AKT2 | | |
| CPT1B | | |
| SERINC1 | | |
| TRAF6 | | |
| IRF9 | | |
| RAI1 | | |
| TAOK2 | | |
| RBX1 | | |
| COX19 | | |
| ADAR | | |
| SERINC3 | | |
| TYROBP | | |
| FASN | | |
| LDHA | | |
| CD244 | | |
| SLC3A2 | | |
| IRF4 | | |
| NBL1 | | |
| HDAC7 | | |
| CD247 | | |
| PFKP | | |
| CYBB | | |
| ACVR1B | | |
| SOCS5 | | |
| HSPA9 | | |
| RDH14 | | |
| SMARCA4 | | |
| NMT1 | | |
| IFNGR1 | | |
| ARAF | | |
| MAX | | |
| PTPRC | | |
| TRBV11-1 | | |
| MX1 | | |
| IGF1R | | |
| TIGIT | | |
| IRF3 | | |
| CCL5 | | |
| GLS | | |
| NDUFA6 | | |
| AURKA | | |
| MTHFR | | |
| CCR4 | | |
| KLRK1 | | |
| TRIM33 | | |
| CDC26 | | |
| HLA-DRA | | |
| IFI6 | | |
| CCR7 | | |
| TKT | | |
| FASLG | | |
| TRAT1 | | |
| CPT1A | | |
| CXCR3 | | |
| HLA-DQA1 | | |
| NRF1 | | |
| BLK | | |
| CD28 | | |
| NAA20 | | |
| PGAM1 | | |
| GNAI2 | | |
| NFATC2IP | | |
| OXSM | | |
| CCNC | | |
| STAM | | |
| SMAD2 | | |
| LAMP2 | | |
| MAML2 | | |
| XAF1 | | |
| GZMH | | |
| ACVR2A | | |
| IL2RB | | |
| MFN2 | | |
| PDK3 | | |
| TRAV10 | | |
| MINOS1 | | |
| IKZF2 | | |
| PSMA2 | | |
| ATP6V1F | | |
| STAT2 | | |
| GRK2 | | |
| MID1IP1 | | |
| DIABLO | | |
| SRR | | |
| STAT1 | | |
| TRAV1-2 | | |
| SOCS2 | | |
| SP100 | | |
| GRPEL1 | | |
| IL6ST | | |
| PDHA1 | | |
| PTGER2 | | |
| MAPK3 | | |
| LTA | | |
| COX4I1 | | |
| TRAV13-1 | | |
| GARS | | |
| MAP2K7 | | |
| ALDH8A1 | | |
| FYN | | |
| RAE1 | | |
| HLA-DRB1 | | |
| TRAV26-2 | | |
| CALM1 | | |
| NFAT5 | | |
| SKP1 | | |
| GFER | | |
| IRF5 | | |
| LAMTOR1 | | |
| ICOS | | |
| TFRC | | |
| NEDD8 | | |
| NKG7 | | |
| PYCR3 | | |
| SPIB | | |
| GADD45B | | |
| TPR | | |
| TRGV4 | | |
| PTGER4 | | |
| COX7A2 | | |
| JAK1 | | |
| PPIA | | |
| TRAV18 | | |
| TSC2 | | |
| HMGCR | | |
| MYC | | |
| PRDM1 | | |
| STK11 | | |
| BCL2 | | |
| IFITM3 | | |
| JAK2 | | |
| ATP5MG | | |
| STAT5B | | |
| TNFRSF10B | | |
| SDHB | | |
| LAT | | |
| NDUFA2 | | |
| GOT1 | | |
| KLRB1 | | |
| PGK1 | | |
| PSMA6 | | |
| NDUFAB1 | | |
| TLR4 | | |
| RAC2 | | |
| UBE2V1 | | |
| PTCD1 | | |
| CISH | | |
| IKBKE | | |
| CXCL10 | | |
| SH3BP2 | | |
| LILRB3 | | |
| IDH3A | | |
| RPTOR | | |
| TIMM17A | | |
| FCGR3A/B | | |
| STAT5A | | |
| PIK3C3 | | |
| TRAV2 | | |
| PSMA3 | | |
| RDH10 | | |
| ERAP2 | | |
| TRAV9-2 | | |
| TRAV13-2 | | |
| DHFR2 | | |
| SERPINB9 | | |
| TIMM23 | | |
| LEF1 | | |
| TRAV8-2 | | |
| TRGC2 | | |
| MMP2 | | |
| CHMP4A | | |
| MIF | | |
| TCF7 | | |
| GNG10 | | |
| ISG15 | | |
| PTK2B | | |
| PTPN6 | | |
| MAP3K14 | | |
| PML | | |
| OPA1 | | |
| SLAMF7 | | |
| RPL3 | | |
| SLC27A3 | | |
| NDUFA1 | | |
| TGFBR2 | | |
| TRAV4 | | |
| CTLA4 | | |
| NCAPD2 | | |
| PFKL | | |
| CD3G | | |
| COX7C | | |
| COX7B | | |
| GZMK | | |
| OAS2 | | |
| TRAV35 | | |
| TRAV36 | | |
| TRIM25 | | |
| SLC2A1 | | |
| ALDOA | | |
| MTHFS | | |
| TRAV19 | | |
| GFM1 | | |
| HLA-E | | |
| TRAV30 | | |
| CHMP3 | | |
| CTSD | | |
| PSMB10 | | |
| GART | | |
| PARP1 | | |
| DHRS4 | | |
| TLR2 | | |
| TRBV29-1 | | |
| TRAV12-1 | | |
| TRAV8-3 | | |
| ACSF2 | | |
| CREB1 | | |
| MAP3K7 | | |
| TFAM | | |
| PPAT | | |
| ATP5PD | | |
| CD40LG | | |
| IL7R | | |
| CMIP | | |
| FOXO1 | | |
| ACSL5 | | |
| CD3D | | |
| GPI | | |
| TRAV8-1 | | |
| TRBV5-6 | | |
| COX6C | | |
| DAP3 | | |
| MAPK12 | | |
| TRDV3 | | |
| TRAV38-1 | | |
| TRAV17 | | |
| USP18 | | |
| CASP3 | | |
| MDH2 | | |
| PIK3R1 | | |
| TRAV14 | | |
| PRICKLE3 | | |
| SLC25A6 | | |
| TRAV12-2 | | |
| ITGAM | | |
| TRAV3 | | |
| TRAV41 | | |
| VSIR | | |
| MCAT | | |
| TRAC | | |
| TRBV6-6 | | |
| TRBV7-4 | | |
| TRBV7-3 | | |
| TRAV23 | | |
| MAGED1 | | |
| COX5B | | |
| TRDC | | |
| CD3E | | |
| ATP5MF | | |
| TRBV14 | | |
| TRGV3/5 | | |
| MR1 | | |
| NME2 | | |
| PECAM1 | | |
| COX6B1 | | |
| FH | | |
| UQCR10 | | |
| IL23R | | |
| IL21R | | |
| TRAV12-3 | | |
| TGFBR1 | | |
| CD274 | | |
| IL6R | | |
| KIR3DL1/2 | | |
| IL12RB1 | | |
| CD4 | | |
| PCK2 | | |
| IFIT1 | | |
| RPL23 | | |
| CD8A | | |
| NCAPG2 | | |
| ACOT2 | | |
| TFB2M | | |
| TRAV34 | | |
| SELL | | |
| TRAV38-2 | | |
| TOMM6 | | |
| UBA5 | | |
| ADORA2A | | |
| TRBV6-2 | | |
| ATG7 | | |
| BID | | |
| SH2D1A | | |
| SLC2A11 | | |
| GZMM | | |
| KLRC1/2 | | |
| TRGC1 | | |
| CCL28 | | |
| PDK1 | | |
| TRAV21 | | |
| TRDV1 | | |
| CCR6 | | |
| USP15 | | |
| PLCG1 | | |
| MTOR | | |
| STAT4 | | |
| SEC22B | | |
| TRGV2 | | |
| TRAV29 | | |
| HSPE1 | | |
| TET2 | | |
| NDUFA4 | | |
| CD200 | | |
| TRBV7-2 | | |
| NDUFB9 | | |
| TP53 | | |
| PYCR2 | | |
| TRBV2 | | |
| PIDD1 | | |
| LCK | | |
| SIK1 | | |
| TNF | | |
| TRBC1/2 | | |
| TRAV26-1 | | |
| TRBV25-1 | | |
| TRAV8-6 | | |
| DUSP1 | | |
| TRBV18 | | |
| NCR3 | | |
| VAV1 | | |
| TFDP1 | | |
| TRAV20 | | |
| TRBV9 | | |
| HACD4 | | |
| COX16 | | |
| TRBV10-3 | | |
| TRGV8 | | |
| TRAV39 | | |
| HDAC8 | | |
| RNASEL | | |
| TRAV25 | | |
| TRBV5-1 | | |
| TRBV28 | | |
| TRAV6 | | |
| TRIM22 | | |
| CTSW | | |
| SCD | | |
| ZAP70 | | |
| SLC25A20 | | |
| DECR1 | | |
| CCL4/L1 | | |
| JUNB | | |
| CXCR5 | | |
| TRBV6-4 | | |
| TRAV16 | | |
| UQCRQ | | |
| TRBV5-5 | | |
| LPAR6 | | |
| TRBV7-9 | | |
| ALDH3A2 | | |
| TRAV22 | | |
| TRBV7-8 | | |
| TRAV24 | | |
| FKBP1A | | |
| SLAMF6 | | |
| NPRL3 | | |
| LTB | | |
| WAS | | |
| TRBV20-1 | | |
| DGLUCY | | |
| CXCL2 | | |
| CD69 | | |
| TRAV27 | | |
| TRBV7-6 | | |
| TRAF5 | | |
| TRBV3-1 | | |
| TRBV4-3 | | |
| HK2 | | |
| OMA1 | | |
| CD84 | | |
| TRIM34 | | |
| IFI35 | | |
| MS4A1 | | |
| ITGB2 | | |
| TRAV5 | | |
| PRKCB | | |
| TRBV4-2 | | |
| TRBV11-2 | | |
| TRBV12-3 | | |
| TRBV4-1 | | |
| SHMT2 | | |
| TRBV10-2 | | |
| ID2 | | |
| UBE2F | | |
| TRBV6-5 | | |
| MTHFD1L | | |
| IL16 | | |
| TNFSF13B | | |
| TIMP1 | | |
| ACTN1 | | |
| TRBV6-1 | | |
| MTHFD1 | | |
| TRBV12-5 | | |
| XCL1/2 | | |
| MAML3 | | |
| PLCB2 | | |
| ZBTB16 | | |
| NEK2 | | |
| TCL1A | | |
| TRBV15 | | |
| DDIT4 | | |
| IFNG | | |
| TRBV13 | | |
| IL18BP | | |
| ACAD10 | | |
| TRBV6-9 | | |
| ALDOC | | |
| TRBV19 | | |
| TRBV27 | | |
| IL32 | | |
| NT5E | | |
| CD96 | | |
| HAVCR2 | | |
| DOCK2 | | |
| CBR4 | | |
| LILRA5 | | |
| CXCR6 | | |
| TOX | | |
| SHMT1 | | |
| GZMA | | |
| RORC | | |
| KLRG1 | | |
| NME1 | | |
| GLS2 | | |
| IL13 | | |
| EGR1 | | |
| KYAT1 | | |
| SMC2 | | |
| TRBV5-4 | | |
| BUB1 | | |
| PCCA | | |
| PHGDH | | |
| LAIR1 | | |

TABLE 14
T cells 24 hr post-delivery (using SOLUPORE ™ delivery method)

| Genes with less | Genes with greater | Genes with greater |
|---|---|---|
| than 2 fold change | than 2 fold change | than 5 fold change |
| ACACA | None | None |
| ACAD10 | | |
| ACADVL | | |
| ACOT2 | | |
| ACSF2 | | |
| ACSL5 | | |
| ACTN1 | | |
| ACVR1B | | |
| ACVR1C | | |
| ACVR2A | | |
| ADAR | | |
| ADD1 | | |
| ADORA2A | | |
| AFDN | | |
| AHR | | |
| AKT1 | | |
| AKT2 | | |
| ALDH3A2 | | |
| ALDH8A1 | | |
| ALDOA | | |
| ALDOC | | |
| APC | | |
| ARAF | | |
| ATG14 | | |
| ATG7 | | |
| ATP5MF | | |
| ATP5MG | | |
| ATP5PD | | |
| ATP6V1F | | |
| AURKA | | |
| BATF3 | | |
| BATF | | |
| BCL2L1 | | |
| BCL2 | | |
| BCL6 | | |
| BID | | |
| BLK | | |
| BTBD6 | | |
| BUB1 | | |
| CALM1 | | |
| CASP3 | | |
| CASP8 | | |
| CBR4 | | |
| CCL22 | | |
| CCL28 | | |
| CCL3/L1 | | |
| CCL4/L1 | | |
| CCL5 | | |
| CCNC | | |
| CCR2 | | |
| CCR4 | | |
| CCR5 | | |
| CCR6 | | |
| CCR7 | | |
| CCR8 | | |
| CD160 | | |
| CD19 | | |
| CD200 | | |
| CD244 | | |
| CD247 | | |
| CD274 | | |
| CD27 | | |
| CD28 | | |
| CD38 | | |
| CD3D | | |
| CD3E | | |
| CD3G | | |
| CD40LG | | |
| CD40 | | |
| CD45R0 | | |
| CD45RA | | |
| CD4 | | |
| CD68 | | |
| CD69 | | |
| CD6 | | |
| CD7 | | |
| CD80 | | |
| CD84 | | |
| CD8A | | |
| CD8B | | |
| CD96 | | |
| CD99 | | |
| CD9 | | |
| CDC26 | | |
| CDC42 | | |
| CDKN1A | | |
| CEACAM1 | | |
| CHMP3 | | |
| CHMP4A | | |
| CISH | | |
| CKAP5 | | |
| CLCF1 | | |
| CMIP | | |
| COX16 | | |
| COX19 | | |
| COX4I1 | | |
| COX5B | | |
| COX6B1 | | |
| COX6C | | |
| COX7A2 | | |
| COX7B | | |
| COX7C | | |
| CPT1A | | |
| CPT1B | | |
| CREB1 | | |
| CS | | |
| CTLA4 | | |
| CTNNA1 | | |
| CTNNB1 | | |
| CTSD | | |
| CTSW | | |
| CX3CR1 | | |
| CXCL8 | | |
| CXCR3 | | |
| CXCR4 | | |
| CXCR5 | | |
| CXCR6 | | |
| CYBB | | |
| DAP3 | | |
| DDIT4 | | |
| DECR1 | | |
| DGLUCY | | |
| DHFR2 | | |
| DHRS4 | | |
| DIABLO | | |
| DLL1 | | |
| DOCK2 | | |
| DUSP1 | | |
| DVL2 | | |
| ENTPD1 | | |
| EOMES | | |
| ERAP2 | | |
| FASLG | | |
| FAS | | |
| FASN | | |
| FCGR3A/B | | |
| FH | | |
| FKBP1A | | |
| FLCN | | |
| FNIP1 | | |
| FOSB | | |
| FOS | | |
| FOXO1 | | |
| FOXP3 | | |
| FYN | | |
| GADD45B | | |
| GARS | | |
| GART | | |
| GATA3 | | |
| GFER | | |
| GFM1 | | |
| GLS2 | | |
| GLS | | |
| GLUD1/2 | | |
| GNAI2 | | |
| GNG10 | | |
| GOT1 | | |
| GPI | | |
| GRK2 | | |
| GRPEL1 | | |
| GZMA | | |
| GZMB | | |
| GZMH | | |
| GZMK | | |
| GZMM | | |
| HACD4 | | |
| HADHB | | |
| HAVCR2 | | |
| HDAC7 | | |
| HDAC8 | | |
| HIF1A | | |
| HK2 | | |
| HLA-DRA | | |
| HLA-DRB1 | | |
| HLA-E | | |
| HMGCR | | |
| HSPA9 | | |
| HSPE1 | | |
| ICAM1 | | |
| ICOSLG | | |
| ICOS | | |
| ID2 | | |
| IDH3A | | |
| IDO1 | | |
| IFI30 | | |
| IFI35 | | |
| IFI6 | | |
| IFIT1 | | |
| IFIT2 | | |
| IFIT3 | | |
| IFITM3 | | |
| IFNAR1 | | |
| IFNG | | |
| IFNGR1 | | |
| IFNGR2 | | |
| IGF1R | | |
| IKBKE | | |
| IKZF2 | | |
| IKZF4 | | |
| IL10RA | | |
| IL12RB1 | | |
| IL12RB2 | | |
| IL15 | | |
| IL16 | | |
| IL18BP | | |
| IL21R | | |
| IL23A | | |
| IL23R | | |
| IL26 | | |
| IL2RA | | |
| IL2RB | | |
| IL2RG | | |
| IL32 | | |
| IL4R | | |
| IL6R | | |
| IL6ST | | |
| IL7R | | |
| IRF1 | | |
| IRF3 | | |
| IRF4 | | |
| IRF5 | | |
| IRF7 | | |
| IRF8 | | |
| IRF9 | | |
| ISG15 | | |
| ITGAM | | |
| ITGB2 | | |
| JAK1 | | |
| JAK2 | | |
| JUNB | | |
| JUN | | |
| KIR3DL1/2 | | |
| KLRB1 | | |
| KLRC1/2 | | |
| KLRD1 | | |
| KLRG1 | | |
| KLRK1 | | |
| KYAT1 | | |
| LAG3 | | |
| LAIR1 | | |
| LAMP1 | | |
| LAMP2 | | |
| LAMTOR1 | | |
| LAT | | |
| LCK | | |
| LDHA | | |
| LEF1 | | |
| LILRB3 | | |
| LPAR6 | | |
| LTA | | |
| LTB | | |
| MAF | | |
| MAGED1 | | |
| MAML2 | | |
| MAML3 | | |
| MAP2K2 | | |
| MAP2K7 | | |
| MAP3K14 | | |
| MAP3K7 | | |
| MAPK3 | | |
| MAX | | |
| MCAT | | |
| MDH2 | | |
| MFN2 | | |
| MID1IP1 | | |
| MIF | | |
| MINOS1 | | |
| MKI67 | | |
| MPC2 | | |
| MR1 | | |
| MS4A1 | | |
| MT2A | | |
| MTCP1 | | |
| MTHFD1L | | |
| MTHFD1 | | |
| MTHFD2 | | |
| MTHFR | | |
| MTHFS | | |
| MTOR | | |
| MX1 | | |
| MYC | | |
| NAA20 | | |
| NBL1 | | |
| NCAPD2 | | |
| NCAPG2 | | |
| NCAPH | | |
| NCR1 | | |
| NCR3 | | |
| NDUFA1 | | |
| NDUFA2 | | |
| NDUFA4 | | |
| NDUFA6 | | |
| NDUFAB1 | | |
| NDUFB9 | | |
| NEDD8 | | |
| NEK2 | | |
| NFAT5 | | |
| NFATC1 | | |
| NFATC2IP | | |
| NFIL3 | | |
| NFKB2 | | |
| NFKBIA | | |
| NKG7 | | |
| NME1 | | |
| NME2 | | |
| NMT1 | | |
| NOTCH1 | | |
| NOTCH2 | | |
| NPRL3 | | |
| NR3C1 | | |
| NRF1 | | |
| NSD2 | | |
| NT5E | | |
| OAS1 | | |
| OAS2 | | |
| OAS3 | | |
| OASL | | |
| OAT | | |
| OMA1 | | |
| OPA1 | | |
| OXSM | | |
| PARP1 | | |
| PCCA | | |
| PCK2 | | |
| PDHA1 | | |
| PDK1 | | |
| PDK3 | | |
| PECAM1 | | |
| PFKFB4 | | |
| PFKL | | |
| PFKP | | |
| PGAM1 | | |
| PGK1 | | |
| PHGDH | | |
| PIDD1 | | |
| PIK3C3 | | |
| PIK3R1 | | |
| PIK3R2 | | |
| PIK3R3 | | |
| PKM | | |
| PLCB2 | | |
| PLCG1 | | |
| PML | | |
| PPARA | | |
| PPARD | | |
| PPAT | | |
| PPIA | | |
| PPP2R5D | | |
| PPP3CA | | |
| PPT2 | | |
| PRDM1 | | |
| PRF1 | | |
| PRICKLE3 | | |
| PRKAB2 | | |
| PRKCB | | |
| PRKCD | | |
| PSAT1 | | |
| PSMA2 | | |
| PSMA3 | | |
| PSMA6 | | |
| PSMB10 | | |
| PTCD1 | | |
| PTGDR2 | | |
| PTGER2 | | |
| PTGER4 | | |
| PTK2B | | |
| PTPN6 | | |
| PTPRC | | |
| PYCR2 | | |
| PYCR3 | | |
| RAC2 | | |
| RAE1 | | |
| RAI1 | | |
| RARG | | |
| RBX1 | | |
| RDH10 | | |
| RDH14 | | |
| RELA | | |
| RNASEL | | |
| RORA | | |
| RORC | | |
| RPL23 | | |
| RPL3 | | |
| RPTOR | | |
| RUNX3 | | |
| SCD | | |
| SDHB | | |
| SEC22B | | |
| SELL | | |
| SELPLG | | |
| SERINC1 | | |
| SERINC3 | | |
| SERPINB9 | | |
| SGK3 | | |
| SGO2 | | |
| SH2D1A | | |
| SH3BP2 | | |
| SHMT1 | | |
| SHMT2 | | |
| SIK1 | | |
| SKP1 | | |
| SLAMF6 | | |
| SLAMF7 | | |
| SLC25A20 | | |
| SLC25A6 | | |
| SLC27A3 | | |
| SLC2A11 | | |
| SLC2A1 | | |
| SLC3A2 | | |
| SLC7A5 | | |
| SMAD2 | | |
| SMAD3 | | |
| SMAD5 | | |
| SMARCA4 | | |
| SMC2 | | |
| SMURF1 | | |
| SOCS2 | | |
| SOCS4 | | |
| SOCS5 | | |
| SOS2 | | |
| SP100 | | |
| SPIB | | |
| SRR | | |
| STAM | | |
| STAT1 | | |
| STAT2 | | |
| STAT3 | | |
| STAT4 | | |
| STAT5A | | |
| STAT5B | | |
| STAT6 | | |
| STK11 | | |
| TAOK2 | | |
| TBX21 | | |
| TCF7 | | |
| TCL1A | | |
| TET2 | | |
| TFAM | | |
| TFB2M | | |
| TFDP1 | | |
| TFRC | | |
| TGFB1 | | |
| TGFBR1 | | |
| TGFBR2 | | |
| TGIF2 | | |
| TICAM1 | | |
| TIGIT | | |
| TIMM17A | | |
| TIMM23 | | |
| TIMP1 | | |
| TKT | | |
| TLR2 | | |
| TNF | | |
| TNFRSF10B | | |
| TNFRSF18 | | |
| TNFRSF4 | | |
| TNFSF13B | | |
| TOLLIP | | |
| TOMM6 | | |
| TOX | | |
| TP53 | | |
| TPR | | |
| TRAC | | |
| TRAF3 | | |
| TRAF5 | | |
| TRAF6 | | |
| TRAT1 | | |
| TRAV10 | | |
| TRAV1-1 | | |
| TRAV12-1 | | |
| TRAV12-2 | | |
| TRAV12-3 | | |
| TRAV1-2 | | |
| TRAV13-1 | | |
| TRAV13-2 | | |
| TRAV14 | | |
| TRAV16 | | |
| TRAV17 | | |
| TRAV18 | | |
| TRAV19 | | |
| TRAV20 | | |
| TRAV21 | | |
| TRAV22 | | |
| TRAV23 | | |
| TRAV24 | | |
| TRAV25 | | |
| TRAV26-1 | | |
| TRAV26-2 | | |
| TRAV27 | | |
| TRAV29 | | |
| TRAV2 | | |
| TRAV30 | | |
| TRAV34 | | |
| TRAV35 | | |
| TRAV36 | | |
| TRAV38-1 | | |
| TRAV38-2 | | |
| TRAV39 | | |
| TRAV3 | | |
| TRAV41 | | |
| TRAV4 | | |
| TRAV5 | | |
| TRAV6 | | |
| TRAV8-1 | | |
| TRAV8-2 | | |
| TRAV8-3 | | |
| TRAV8-6 | | |
| TRAV9-2 | | |
| TRBC1/2 | | |
| TRBV10-2 | | |
| TRBV10-3 | | |
| TRBV11-1 | | |
| TRBV11-2 | | |
| TRBV11-3 | | |
| TRBV12-3 | | |
| TRBV12-5 | | |
| TRBV13 | | |
| TRBV14 | | |
| TRBV15 | | |
| TRBV18 | | |
| TRBV19 | | |
| TRBV20-1 | | |
| TRBV24-1 | | |
| TRBV25-1 | | |
| TRBV27 | | |
| TRBV28 | | |
| TRBV29-1 | | |
| TRBV2 | | |
| TRBV3-1 | | |
| TRBV4-1 | | |
| TRBV4-2 | | |
| TRBV5-1 | | |
| TRBV5-4 | | |
| TRBV5-5 | | |
| TRBV5-6 | | |
| TRBV6-1 | | |
| TRBV6-2 | | |
| TRBV6-4 | | |
| TRBV6-5 | | |
| TRBV6-6 | | |
| TRBV6-9 | | |
| TRBV7-2 | | |
| TRBV7-3 | | |
| TRBV7-4 | | |
| TRBV7-6 | | |
| TRBV7-8 | | |
| TRBV7-9 | | |
| TRBV9 | | |
| TRDC | | |
| TRDV1 | | |
| TRDV3 | | |
| TRGC1 | | |
| TRGC2 | | |
| TRGV2 | | |
| TRGV3/5 | | |
| TRGV4 | | |
| TRGV8 | | |
| TRIM22 | | |
| TRIM25 | | |
| TRIM26 | | |
| TRIM33 | | |
| TRIM34 | | |
| TSC2 | | |
| TYK2 | | |
| TYROBP | | |
| UBA5 | | |
| UBE2F | | |
| UBE2I | | |
| UBE2V1 | | |
| UQCR10 | | |
| UQCRQ | | |
| USP15 | | |
| USP18 | | |
| VAV1 | | |
| VAV3 | | |
| VSIR | | |
| WAS | | |
| WDR45 | | |
| XAF1 | | |
| XCL1/2 | | |
| ZAP70 | | |
| ZBTB16 | | |

TABLE 15
T cells 6 hr post-Nucleofection (FI-115) Delivery

| Genes with less | Genes with greater | Genes with greater |
|---|---|---|
| than 2 fold change | than 2 fold change | than 5 fold change |
| ACACA | ACAD10 | ACSF2 |
| ACADVL | ACSF2 | ACTN1 |
| ACOT2 | ACSL5 | BATF3 |
| ACVR1B | ACTN1 | CCL3/L1 |
| ACVR1C | AKT1 | CCR2 |
| ACVR2A | ALDH3A2 | CCR5 |
| ADAR | ALDOC | CD200 |
| ADD1 | ATP5PD | CD40LG |
| ADORA2A | BATF | CISH |
| AFDN | BATF3 | CXCL8 |
| AHR | BID | CXCR6 |
| AKT2 | BUB1 | FOS |
| ALDH8A1 | CASP8 | FOSB |
| ALDOA | CBR4 | GZMA |
| APC | CCL3/L1 | IFI30 |
| ARAF | CCL4/L1 | IFIT3 |
| ATG14 | CCR2 | IFNG |
| ATG7 | CCR5 | IL16 |
| ATP5MF | CCR6 | IL2 |
| ATP5MG | CCR7 | IL32 |
| ATP6V1F | CD160 | IRF4 |
| AURKA | CD200 | NCR3 |
| BCL2 | CD4 | PECAM1 |
| BCL2L1 | CD40LG | PTGDR2 |
| BCL6 | CD68 | SGO2 |
| BLK | CD69 | SIK1 |
| BTBD6 | CD80 | SLC27A3 |
| CALM1 | CD8A | SLC7A5 |
| CASP3 | CD8B | TIMP1 |
| CCL20 | CD96 | TRAV1-1 |
| CCL28 | CISH | TRBV4-1 |
| CCL5 | CLCF1 | TRBV6-1 |
| CCNC | CMIP | |
| CCR4 | COX5B | |
| CD244 | CTLA4 | |
| CD247 | CTSD | |
| CD27 | CTSW | |
| CD274 | CX3CR1 | |
| CD28 | CXCL10 | |
| CD3D | CXCL8 | |
| CD3E | CXCR3 | |
| CD3G | CXCR6 | |
| CD40 | CYBB | |
| CD45R0 | DECR1 | |
| CD45RA | DGLUCY | |
| CD6 | DHRS4 | |
| CD7 | DLL1 | |
| CD84 | DOCK2 | |
| CD9 | DUSP1 | |
| CD99 | EGR1 | |
| CDC26 | FASLG | |
| CDC42 | FCGR3A/B | |
| CDKN1A | FOS | |
| CHMP3 | FOSB | |
| CHMP4A | FOXP3 | |
| CKAP5 | FYN | |
| COX16 | GARS | |
| COX19 | GLS | |
| COX4I1 | GLS2 | |
| COX6B1 | GLUD1/2 | |
| COX6C | GNAI2 | |
| COX7A2 | GNG10 | |
| COX7B | GPI | |
| COX7C | GRK2 | |
| CPT1A | GZMA | |
| CPT1B | GZMH | |
| CREB1 | GZMK | |
| CS | GZMM | |
| CTNNA1 | HACD4 | |
| CTNNB1 | HAVCR2 | |
| CXCL2 | HDAC7 | |
| CXCR4 | HIF1A | |
| CXCR5 | HK2 | |
| DAP3 | HLA-DRA | |
| DDIT4 | ICOS | |
| DHFR2 | IFI30 | |
| DIABLO | IFI35 | |
| DVL2 | IFI6 | |
| EOMES | IFIT3 | |
| ERAP2 | IFITM3 | |
| FAS | IFNG | |
| FASN | IKBKE | |
| FH | IKZF4 | |
| FKBP1A | IL10RA | |
| FLCN | IL12RB1 | |
| FNIP1 | IL12RB2 | |
| FOXO1 | IL16 | |
| GADD45B | IL18BP | |
| GART | IL2 | |
| GATA3 | IL21R | |
| GFER | IL32 | |
| GFM1 | IL36A | |
| GOT1 | IL7R | |
| GRPEL1 | IRF3 | |
| GZMB | IRF4 | |
| HADHB | IRF5 | |
| HDAC8 | ISG15 | |
| HLA-DQA1 | ITGB2 | |
| HLA-DRB1 | JUN | |
| HLA-E | KIR3DL1/2 | |
| HMGCR | KLRD1 | |
| HSPA9 | KYAT1 | |
| HSPE1 | LAIR1 | |
| ICAM1 | LAT | |
| ICOSLG | LCK | |
| ID2 | LTB | |
| IDH3A | MAGED1 | |
| IDO1 | MAP2K2 | |
| IFIT1 | MAP2K7 | |
| IFIT2 | MAPK3 | |
| IFNAR1 | MR1 | |
| IFNGR1 | MS4A1 | |
| IFNGR2 | MTHFD1 | |
| IGF1R | MTHFD1L | |
| IKZF2 | MTHFD2 | |
| IL13 | MTHFR | |
| IL15 | NBL1 | |
| IL23A | NCAPD2 | |
| IL23R | NCAPG2 | |
| IL2RA | NCR3 | |
| IL2RB | NEDD8 | |
| IL2RG | NFATC1 | |
| IL4R | NFIL3 | |
| IL6R | NFKB2 | |
| IL6ST | NFKBIA | |
| IRF1 | NKG7 | |
| IRF7 | NOTCH1 | |
| IRF9 | NPRL3 | |
| ITGAM | OAS1 | |
| JAK1 | OAS2 | |
| JAK2 | OMA1 | |
| JUNB | PARP1 | |
| KLRB1 | PCCA | |
| KLRC1/2 | PECAM1 | |
| KLRG1 | PFKL | |
| KLRK1 | PGAM1 | |
| LAG3 | PIK3R2 | |
| LAMP1 | PLCB2 | |
| LAMP2 | PLCG1 | |
| LAMTOR1 | PPP2R5D | |
| LDHA | PPT2 | |
| LEF1 | PRF1 | |
| LILRA5 | PRICKLE3 | |
| LILRB3 | PRKCB | |
| LPAR6 | PSMB10 | |
| LTA | PTGDR2 | |
| MAF | PTGER4 | |
| MAML2 | RAC2 | |
| MAML3 | RAI1 | |
| MAP3K14 | RNASEL | |
| MAP3K7 | RPTOR | |
| MAPK12 | SCD | |
| MAX | SELL | |
| MCAT | SELPLG | |
| MDH2 | SGO2 | |
| MFN2 | SH2D1A | |
| MID1IP1 | SH3BP2 | |
| MIF | SHMT1 | |
| MINOS1 | SIK1 | |
| MMP2 | SLC25A20 | |
| MPC2 | SLC27A3 | |
| MT2A | SLC2A11 | |
| MTCP1 | SLC3A2 | |
| MTHFS | SLC7A5 | |
| MTOR | SMAD3 | |
| MX1 | SMC2 | |
| MYC | SRR | |
| NAA20 | STAT3 | |
| NCAPH | STAT5A | |
| NCR1 | STK11 | |
| NDUFA1 | TBX21 | |
| NDUFA2 | TCF7 | |
| NDUFA4 | TFDP1 | |
| NDUFA6 | TFRC | |
| NDUFAB1 | TGFBR1 | |
| NDUFB9 | TIMP1 | |
| NEK2 | TNFSF13B | |
| NFAT5 | TRAC | |
| NFATC2IP | TRAT1 | |
| NME1 | TRAV10 | |
| NME2 | TRAV1-1 | |
| NMT1 | TRAV1-2 | |
| NOTCH2 | TRAV12-1 | |
| NR3C1 | TRAV12-2 | |
| NRF1 | TRAV12-3 | |
| NSD2 | TRAV13-2 | |
| NT5E | TRAV14 | |
| OAS3 | TRAV17 | |
| OASL | TRAV18 | |
| OAT | TRAV20 | |
| OPA1 | TRAV21 | |
| OXSM | TRAV22 | |
| PCK2 | TRAV23 | |
| PDHA1 | TRAV24 | |
| PDK1 | TRAV25 | |
| PDK3 | TRAV26-1 | |
| PFKP | TRAV26-2 | |
| PGK1 | TRAV27 | |
| PHGDH | TRAV29 | |
| PIDD1 | TRAV3 | |
| PIK3C3 | TRAV30 | |
| PIK3R1 | TRAV34 | |
| PKM | TRAV35 | |
| PML | TRAV38-1 | |
| PPARA | TRAV38-2 | |
| PPAT | TRAV39 | |
| PPIA | TRAV4 | |
| PPP3CA | TRAV41 | |
| PRDM1 | TRAV5 | |
| PRKAB2 | TRAV6 | |
| PRKCD | TRAV8-1 | |
| PSAT1 | TRAV8-3 | |
| PSMA2 | TRAV8-6 | |
| PSMA3 | TRAV9-2 | |
| PSMA6 | TRBC1/2 | |
| PTCD1 | TRBV10-2 | |
| PTGER2 | TRBV10-3 | |
| PTK2B | TRBV11-1 | |
| PTPN6 | TRBV11-2 | |
| PTPRC | TRBV12-3 | |
| PYCR2 | TRBV12-5 | |
| PYCR3 | TRBV13 | |
| RAE1 | TRBV14 | |
| RARG | TRBV15 | |
| RBX1 | TRBV18 | |
| RDH10 | TRBV19 | |
| RDH14 | TRBV2 | |
| RELA | TRBV20-1 | |
| RORA | TRBV25-1 | |
| RORC | TRBV27 | |
| RPL23 | TRBV28 | |
| RPL3 | TRBV29-1 | |
| RUNX3 | TRBV30 | |
| SDHB | TRBV3-1 | |
| SEC22B | TRBV4-1 | |
| SERINC1 | TRBV4-2 | |
| SERINC3 | TRBV4-3 | |
| SERPINB9 | TRBV5-1 | |
| SGK3 | TRBV5-4 | |
| SHMT2 | TRBV5-5 | |
| SKP1 | TRBV5-6 | |
| SLAMF6 | TRBV6-1 | |
| SLAMF7 | TRBV6-2 | |
| SLC25A6 | TRBV6-4 | |
| SLC2A1 | TRBV6-5 | |
| SMAD2 | TRBV6-6 | |
| SMAD5 | TRBV6-9 | |
| SMARCA4 | TRBV7-2 | |
| SMURF1 | TRBV7-3 | |
| SOCS2 | TRBV7-6 | |
| SOCS4 | TRBV7-8 | |
| SOCS5 | TRBV7-9 | |
| SOS2 | TRBV9 | |
| SP100 | TRDC | |
| SPIB | TRDV1 | |
| STAM | TRGC1 | |
| STAT1 | TRGV4 | |
| STAT2 | TRGV8 | |
| STAT4 | TRIM34 | |
| STAT5B | TYROBP | |
| STAT6 | USP18 | |
| TAOK2 | VAV1 | |
| TCL1A | VSIR | |
| TET2 | WAS | |
| TFAM | XCL1/2 | |
| TFB2M | | |
| TGFB1 | | |
| TGFBR2 | | |
| TGIF2 | | |
| TICAM1 | | |
| TIGIT | | |
| TIMM17A | | |
| TIMM23 | | |
| TKT | | |
| TLR2 | | |
| TLR4 | | |
| TNF | | |
| TNFRSF10B | | |
| TNFRSF18 | | |
| TNFRSF4 | | |
| TOLLIP | | |
| TOMM6 | | |
| TOX | | |
| TP53 | | |
| TPR | | |
| TRAF3 | | |
| TRAF5 | | |
| TRAF6 | | |
| TRAV13-1 | | |
| TRAV16 | | |
| TRAV19 | | |
| TRAV2 | | |
| TRAV36 | | |
| TRAV8-2 | | |
| TRBV7-4 | | |
| TRDV3 | | |
| TRGC2 | | |
| TRGV2 | | |
| TRGV3/5 | | |
| TRIM22 | | |
| TRIM25 | | |
| TRIM26 | | |
| TRIM33 | | |
| TSC2 | | |
| TYK2 | | |
| UBA5 | | |
| UBE2F | | |
| UBE2I | | |
| UBE2V1 | | |
| UQCR10 | | |
| UQCRQ | | |
| USP15 | | |
| VAV3 | | |
| WDR45 | | |
| XAF1 | | |
| ZAP70 | | |
| ZBTB16 | | |

TABLE 16
T cells 24 hr post-Nucleofection (FI-115) Delivery

| Genes with less | Genes with greater | Genes with greater |
|---|---|---|
| than 2 fold change | than 2 fold change | than 5 fold change |
| ACACA | AHR | CD200 |
| ACAD10 | BATF | FOSB |
| ACADVL | BATF3 | CD38 |
| ACOT2 | CCL22 | FCGR3A/B |
| ACSF2 | CCL4/L1 | CX3CR1 |
| ACSL5 | CD19 | |
| ACTN1 | CD200 | |
| ACVR1B | CD244 | |
| ACVR1C | CD38 | |
| ACVR2A | CD68 | |
| ADAR | CTSW | |
| ADD1 | CX3CR1 | |
| ADORA2A | FCGR3A/B | |
| AFDN | FOS | |
| AKT1 | FOSB | |
| AKT2 | GZMA | |
| ALDH3A2 | GZMH | |
| ALDH8A1 | GZMK | |
| ALDOA | ICOSLG | |
| ALDOC | IFIT3 | |
| APC | IL12RB2 | |
| ARAF | IL7R | |
| ATG14 | IRF4 | |
| ATG7 | IRF8 | |
| ATP5MF | ITGAM | |
| ATP5MG | JUN | |
| ATP5PD | KLRB1 | |
| ATP6V1F | LAIR1 | |
| AURKA | MT2A | |
| BCL2 | NCR1 | |
| BCL2L1 | NFIL3 | |
| BCL6 | NT5E | |
| BID | PRF1 | |
| BLK | SELL | |
| BTBD6 | TIMP1 | |
| BUB1 | TRGC2 | |
| CALM1 | TRGV2 | |
| CASP3 | TYROBP | |
| CASP8 | | |
| CBR4 | | |
| CCL28 | | |
| CCL3/L1 | | |
| CCL5 | | |
| CCNC | | |
| CCR2 | | |
| CCR4 | | |
| CCR5 | | |
| CCR6 | | |
| CCR7 | | |
| CCR8 | | |
| CD160 | | |
| CD247 | | |
| CD27 | | |
| CD274 | | |
| CD28 | | |
| CD3D | | |
| CD3E | | |
| CD3G | | |
| CD4 | | |
| CD40 | | |
| CD40LG | | |
| CD45R0 | | |
| CD45RA | | |
| CD6 | | |
| CD69 | | |
| CD7 | | |
| CD80 | | |
| CD84 | | |
| CD8A | | |
| CD8B | | |
| CD9 | | |
| CD96 | | |
| CD99 | | |
| CDC26 | | |
| CDC42 | | |
| CDKN1A | | |
| CEACAM1 | | |
| CHMP3 | | |
| CHMP4A | | |
| CISH | | |
| CKAP5 | | |
| CLCF1 | | |
| CMIP | | |
| COX16 | | |
| COX19 | | |
| COX4I1 | | |
| COX5B | | |
| COX6B1 | | |
| COX6C | | |
| COX7A2 | | |
| COX7B | | |
| COX7C | | |
| CPT1A | | |
| CPT1B | | |
| CREB1 | | |
| CS | | |
| CTLA4 | | |
| CTNNA1 | | |
| CTNNB1 | | |
| CTSD | | |
| CXCL8 | | |
| CXCR3 | | |
| CXCR4 | | |
| CXCR5 | | |
| CXCR6 | | |
| CYBB | | |
| DAP3 | | |
| DDIT4 | | |
| DECR1 | | |
| DGLUCY | | |
| DHFR2 | | |
| DHRS4 | | |
| DIABLO | | |
| DLL1 | | |
| DOCK2 | | |
| DUSP1 | | |
| DVL2 | | |
| ENTPD1 | | |
| EOMES | | |
| ERAP2 | | |
| FAS | | |
| FASLG | | |
| FASN | | |
| FH | | |
| FKBP1A | | |
| FLCN | | |
| FNIP1 | | |
| FOXO1 | | |
| FOXP3 | | |
| FYN | | |
| GADD45B | | |
| GARS | | |
| GART | | |
| GATA3 | | |
| GFER | | |
| GFM1 | | |
| GLS | | |
| GLS2 | | |
| GLUD1/2 | | |
| GNAI2 | | |
| GNG10 | | |
| GOT1 | | |
| GPI | | |
| GRK2 | | |
| GRPEL1 | | |
| GZMB | | |
| GZMM | | |
| HACD4 | | |
| HADHB | | |
| HAVCR2 | | |
| HDAC7 | | |
| HDAC8 | | |
| HIF1A | | |
| HK2 | | |
| HLA-DRA | | |
| HLA-DRB1 | | |
| HLA-E | | |
| HMGCR | | |
| HSPA9 | | |
| HSPE1 | | |
| ICAM1 | | |
| ICOS | | |
| ID2 | | |
| IDH3A | | |
| IDO1 | | |
| IFI30 | | |
| IFI35 | | |
| IFI6 | | |
| IFIT1 | | |
| IFIT2 | | |
| IFITM3 | | |
| IFNAR1 | | |
| IFNG | | |
| IFNGR1 | | |
| IFNGR2 | | |
| IGF1R | | |
| IKBKE | | |
| IKZF2 | | |
| IKZF4 | | |
| IL10RA | | |
| IL12RB1 | | |
| IL15 | | |
| IL16 | | |
| IL18BP | | |
| IL21R | | |
| IL23A | | |
| IL23R | | |
| IL26 | | |
| IL2RA | | |
| IL2RB | | |
| IL2RG | | |
| IL32 | | |
| IL4R | | |
| IL6R | | |
| IL6ST | | |
| IRF1 | | |
| IRF3 | | |
| IRF5 | | |
| IRF7 | | |
| IRF9 | | |
| ISG15 | | |
| ITGB2 | | |
| JAK1 | | |
| JAK2 | | |
| JUNB | | |
| KIR3DL1/2 | | |
| KLRC1/2 | | |
| KLRD1 | | |
| KLRG1 | | |
| KLRK1 | | |
| KYAT1 | | |
| LAG3 | | |
| LAMP1 | | |
| LAMP2 | | |
| LAMTOR1 | | |
| LAT | | |
| LCK | | |
| LDHA | | |
| LEF1 | | |
| LILRB3 | | |
| LPAR6 | | |
| LTA | | |
| LTB | | |
| MAF | | |
| MAGED1 | | |
| MAML2 | | |
| MAML3 | | |
| MAP2K2 | | |
| MAP2K7 | | |
| MAP3K14 | | |
| MAP3K7 | | |
| MAPK3 | | |
| MAX | | |
| MCAT | | |
| MDH2 | | |
| MFN2 | | |
| MID1IP1 | | |
| MIF | | |
| MINOS1 | | |
| MKI67 | | |
| MPC2 | | |
| MR1 | | |
| MS4A1 | | |
| MTCP1 | | |
| MTHFD1 | | |
| MTHFD1L | | |
| MTHFD2 | | |
| MTHFR | | |
| MTHFS | | |
| MTOR | | |
| MX1 | | |
| MYC | | |
| NAA20 | | |
| NBL1 | | |
| NCAPD2 | | |
| NCAPG2 | | |
| NCAPH | | |
| NCR3 | | |
| NDUFA1 | | |
| NDUFA2 | | |
| NDUFA4 | | |
| NDUFA6 | | |
| NDUFAB1 | | |
| NDUFB9 | | |
| NEDD8 | | |
| NEK2 | | |
| NFAT5 | | |
| NFATC1 | | |
| NFATC2IP | | |
| NFKB2 | | |
| NFKBIA | | |
| NKG7 | | |
| NME1 | | |
| NME2 | | |
| NMT1 | | |
| NOTCH1 | | |
| NOTCH2 | | |
| NPRL3 | | |
| NR3C1 | | |
| NRF1 | | |
| NSD2 | | |
| OAS1 | | |
| OAS2 | | |
| OAS3 | | |
| OASL | | |
| OAT | | |
| OMA1 | | |
| OPAl | | |
| OXSM | | |
| PARP1 | | |
| PCCA | | |
| PCK2 | | |
| PDHA1 | | |
| PDK1 | | |
| PDK3 | | |
| PECAM1 | | |
| PFKFB4 | | |
| PFKL | | |
| PFKP | | |
| PGAM1 | | |
| PGK1 | | |
| PHGDH | | |
| PIDD1 | | |
| PIK3C3 | | |
| PIK3R1 | | |
| PIK3R2 | | |
| PIK3R3 | | |
| PKM | | |
| PLCB2 | | |
| PLCG1 | | |
| PML | | |
| PPARA | | |
| PPARD | | |
| PPAT | | |
| PPIA | | |
| PPP2R5D | | |
| PPP3CA | | |
| PPT2 | | |
| PRDM1 | | |
| PRICKLE3 | | |
| PRKAB2 | | |
| PRKCB | | |
| PRKCD | | |
| PSAT1 | | |
| PSMA2 | | |
| PSMA3 | | |
| PSMA6 | | |
| PSMB10 | | |
| PTCD1 | | |
| PTGDR2 | | |
| PTGER2 | | |
| PTGER4 | | |
| PTK2B | | |
| PTPN6 | | |
| PTPRC | | |
| PYCR2 | | |
| PYCR3 | | |
| RAC2 | | |
| RAE1 | | |
| RAI1 | | |
| RARG | | |
| RBX1 | | |
| RDH10 | | |
| RDH14 | | |
| RELA | | |
| RNASEL | | |
| RORA | | |
| RORC | | |
| RPL23 | | |
| RPL3 | | |
| RPTOR | | |
| RUNX3 | | |
| SCD | | |
| SDHB | | |
| SEC22B | | |
| SELPLG | | |
| SERINC1 | | |
| SERINC3 | | |
| SERPINB9 | | |
| SGK3 | | |
| SGO2 | | |
| SH2D1A | | |
| SH3BP2 | | |
| SHMT1 | | |
| SHMT2 | | |
| SIK1 | | |
| SKP1 | | |
| SLAMF6 | | |
| SLAMF7 | | |
| SLC25A20 | | |
| SLC25A6 | | |
| SLC27A3 | | |
| SLC2A1 | | |
| SLC2A11 | | |
| SLC3A2 | | |
| SLC7A5 | | |
| SMAD2 | | |
| SMAD3 | | |
| SMAD5 | | |
| SMARCA4 | | |
| SMC2 | | |
| SMURF1 | | |
| SOCS2 | | |
| SOCS4 | | |
| SOCS5 | | |
| SOS2 | | |
| SP100 | | |
| SPIB | | |
| SRR | | |
| STAM | | |
| STAT1 | | |
| STAT2 | | |
| STAT3 | | |
| STAT4 | | |
| STAT5A | | |
| STAT5B | | |
| STAT6 | | |
| STK11 | | |
| TAOK2 | | |
| TBX21 | | |
| TCF7 | | |
| TCL1A | | |
| TET2 | | |
| TFAM | | |
| TFB2M | | |
| TFDP1 | | |
| TFRC | | |
| TGFB1 | | |
| TGFBR1 | | |
| TGFBR2 | | |
| TGIF2 | | |
| TICAM1 | | |
| TIGIT | | |
| TIMM17A | | |
| TIMM23 | | |
| TKT | | |
| TLR2 | | |
| TNF | | |
| TNFRSF10B | | |
| TNFRSF18 | | |
| TNFRSF4 | | |
| TNFSF13B | | |
| TOLLIP | | |
| TOMM6 | | |
| TOX | | |
| TP53 | | |
| TPR | | |
| TRAC | | |
| TRAF3 | | |
| TRAF5 | | |
| TRAF6 | | |
| TRAT1 | | |
| TRAV10 | | |
| TRAV1-1 | | |
| TRAV1-2 | | |
| TRAV12-1 | | |
| TRAV12-2 | | |
| TRAV12-3 | | |
| TRAV13-1 | | |
| TRAV13-2 | | |
| TRAV14 | | |
| TRAV16 | | |
| TRAV17 | | |
| TRAV18 | | |
| TRAV19 | | |
| TRAV2 | | |
| TRAV20 | | |
| TRAV21 | | |
| TRAV22 | | |
| TRAV23 | | |
| TRAV24 | | |
| TRAV25 | | |
| TRAV26-1 | | |
| TRAV26-2 | | |
| TRAV27 | | |
| TRAV29 | | |
| TRAV3 | | |
| TRAV30 | | |
| TRAV34 | | |
| TRAV35 | | |
| TRAV36 | | |
| TRAV38-1 | | |
| TRAV38-2 | | |
| TRAV39 | | |
| TRAV4 | | |
| TRAV41 | | |
| TRAV5 | | |
| TRAV6 | | |
| TRAV8-1 | | |
| TRAV8-2 | | |
| TRAV8-3 | | |
| TRAV8-6 | | |
| TRAV9-2 | | |
| TRBC1/2 | | |
| TRBV10-2 | | |
| TRBV10-3 | | |
| TRBV11-1 | | |
| TRBV11-2 | | |
| TRBV11-3 | | |
| TRBV12-3 | | |
| TRBV12-5 | | |
| TRBV13 | | |
| TRBV14 | | |
| TRBV15 | | |
| TRBV18 | | |
| TRBV19 | | |
| TRBV2 | | |
| TRBV20-1 | | |
| TRBV24-1 | | |
| TRBV25-1 | | |
| TRBV27 | | |
| TRBV28 | | |
| TRBV29-1 | | |
| TRBV3-1 | | |
| TRBV4-1 | | |
| TRBV4-2 | | |
| TRBV5-1 | | |
| TRBV5-4 | | |
| TRBV5-5 | | |
| TRBV5-6 | | |
| TRBV6-1 | | |
| TRBV6-2 | | |
| TRBV6-4 | | |
| TRBV6-5 | | |
| TRBV6-6 | | |
| TRBV6-9 | | |
| TRBV7-2 | | |
| TRBV7-3 | | |
| TRBV7-4 | | |
| TRBV7-6 | | |
| TRBV7-8 | | |
| TRBV7-9 | | |
| TRBV9 | | |
| TRDC | | |
| TRDV1 | | |
| TRDV3 | | |
| TRGC1 | | |
| TRGV3/5 | | |
| TRGV4 | | |
| TRGV8 | | |
| TRIM22 | | |
| TRIM25 | | |
| TRIM26 | | |
| TRIM33 | | |
| TRIM34 | | |
| TSC2 | | |
| TYK2 | | |
| UBA5 | | |
| UBE2F | | |
| UBE2I | | |
| UBE2V1 | | |
| UQCR10 | | |
| UQCRQ | | |
| USP15 | | |
| USP18 | | |
| VAV1 | | |
| VAV3 | | |
| VSIR | | |
| WAS | | |
| WDR45 | | |
| XAF1 | | |
| XCL1/2 | | |
| ZAP70 | | |
| ZBTB16 | | |

TABLE 17
T cells 24 hr post-Nucleofection (EO-115) Delivery

| Genes with less | Genes with greater | Genes with greater |
|---|---|---|
| than 2 fold change | than 2 fold change | than 5 fold change |
| IRF8 | FOSB | FOSB |
| IRF4 | CD200 | |
| AHR | JUN | |
| BATF | FOS | |
| CD27 | CD68 | |
| STAT1 | CCL22 | |
| SLC3A2 | BATF3 | |
| FLCN | CD19 | |
| RPTOR | CD38 | |
| LAT | CD9 | |
| CXCR4 | ICOSLG | |
| IL12RB2 | MT2A | |
| PHGDH | IFIT1 | |
| SLAMF7 | CCL3/L1 | |
| NFIL3 | CCR8 | |
| SH2D1A | | |
| CDKN1A | | |
| CTLA4 | | |
| FNIP1 | | |
| FKBP1A | | |
| CTNNA1 | | |
| PKM | | |
| TRAV30 | | |
| RDH10 | | |
| TRAV13-1 | | |
| MAF | | |
| IFNGR2 | | |
| SMAD3 | | |
| SCD | | |
| TRBV15 | | |
| TRBV13 | | |
| GADD45B | | |
| SOS2 | | |
| NCAPH | | |
| PFKP | | |
| TRAV34 | | |
| IFIT3 | | |
| PFKFB4 | | |
| TRAV19 | | |
| TRBV12-3 | | |
| CASP8 | | |
| TRAV2 | | |
| ACSL5 | | |
| ICAM1 | | |
| IRF9 | | |
| IL26 | | |
| IL15 | | |
| ACVR1C | | |
| TRBV6-6 | | |
| DUSP1 | | |
| CD8B | | |
| BCL6 | | |
| GZMB | | |
| TRBV3-1 | | |
| PSAT1 | | |
| TRAV36 | | |
| TRBV25-1 | | |
| IRF5 | | |
| MTHFD2 | | |
| IFI35 | | |
| MS4A1 | | |
| CPT1B | | |
| TRAV29 | | |
| TRBV4-2 | | |
| TNFSF13B | | |
| TRBV10-3 | | |
| TRDC | | |
| TCL1A | | |
| HMGCR | | |
| USP18 | | |
| TRAV21 | | |
| TRAV17 | | |
| TRAC | | |
| TRAV4 | | |
| TRBV18 | | |
| TRBV6-9 | | |
| DVL2 | | |
| IRF7 | | |
| TRAV12-3 | | |
| NOTCH2 | | |
| TRAV23 | | |
| TRAV26-1 | | |
| ACADVL | | |
| TRBV6-5 | | |
| PGK1 | | |
| ATP6V1F | | |
| DDIT4 | | |
| TRBV4-1 | | |
| DHFR2 | | |
| SPIB | | |
| STAT3 | | |
| CD28 | | |
| TRAV10 | | |
| MTCP1 | | |
| SLC7A5 | | |
| ALDOC | | |
| CD6 | | |
| PIK3R3 | | |
| NDUFA2 | | |
| TRBV28 | | |
| NFKB2 | | |
| BTBD6 | | |
| SIK1 | | |
| TRBV27 | | |
| TFRC | | |
| IL2RB | | |
| LAMTOR1 | | |
| CCL5 | | |
| TRAV14 | | |
| PRKCD | | |
| TRAV24 | | |
| IRF1 | | |
| TRAV20 | | |
| TRBV12-5 | | |
| TRBV7-9 | | |
| ISG15 | | |
| TRBV6-1 | | |
| CD69 | | |
| TRAV27 | | |
| ACAD10 | | |
| MAP3K14 | | |
| TRBV9 | | |
| NBL1 | | |
| MDH2 | | |
| TRAV5 | | |
| TRAV6 | | |
| TRAV35 | | |
| ALDOA | | |
| TRBC1/2 | | |
| HSPE1 | | |
| TRDV1 | | |
| ID2 | | |
| NDUFA6 | | |
| NFATC2IP | | |
| TOLLIP | | |
| UQCR10 | | |
| MPC2 | | |
| PTGDR2 | | |
| TKT | | |
| DGLUCY | | |
| TRBV2 | | |
| TNFRSF4 | | |
| MAPK3 | | |
| FAS | | |
| TRAV1-1 | | |
| BUB1 | | |
| TRBV24-1 | | |
| TRAV12-2 | | |
| NDUFA4 | | |
| SP100 | | |
| DHRS4 | | |
| FASN | | |
| IL23A | | |
| TRAV8-3 | | |
| CCL28 | | |
| TRAV39 | | |
| TRAV26-2 | | |
| GARS | | |
| CD7 | | |
| MTHFR | | |
| TRAV41 | | |
| TRBV20-1 | | |
| COX5B | | |
| TRAV12-1 | | |
| CD99 | | |
| TRBV6-2 | | |
| GOT1 | | |
| MAP2K2 | | |
| SRR | | |
| GPI | | |
| TRAV9-2 | | |
| TRBV7-8 | | |
| CD40 | | |
| MTHFD1L | | |
| COX6C | | |
| RUNX3 | | |
| TRAV38-2 | | |
| LDHA | | |
| PIK3R2 | | |
| STAT2 | | |
| TYK2 | | |
| TRAV38-1 | | |
| PTCD1 | | |
| TRBV5-5 | | |
| GLS | | |
| DAP3 | | |
| TRIM22 | | |
| PPIA | | |
| NFAT5 | | |
| COX7A2 | | |
| TRAV13-2 | | |
| STAT6 | | |
| CBR4 | | |
| LAMP2 | | |
| NEDD8 | | |
| SGO2 | | |
| TAOK2 | | |
| CCR7 | | |
| CD247 | | |
| PFKL | | |
| TRAV25 | | |
| TRBV5-1 | | |
| TRBV5-4 | | |
| MIF | | |
| CHMP3 | | |
| GFM1 | | |
| TOMM6 | | |
| HADHB | | |
| ATP5PD | | |
| CD96 | | |
| TFB2M | | |
| LAMP1 | | |
| PDK3 | | |
| FOXP3 | | |
| TRAV22 | | |
| IL2RA | | |
| TRAF6 | | |
| TRBV5-6 | | |
| OMA1 | | |
| SHMT1 | | |
| TRAV16 | | |
| MFN2 | | |
| CCNC | | |
| COX6B1 | | |
| TRBV7-6 | | |
| COX4I1 | | |
| PGAM1 | | |
| DECR1 | | |
| CHMP4A | | |
| GNG10 | | |
| COX7B | | |
| TRBV7-2 | | |
| TRAV8-6 | | |
| APC | | |
| PCK2 | | |
| TIGIT | | |
| RAC2 | | |
| MAGED1 | | |
| PPARA | | |
| HIF1A | | |
| SMC2 | | |
| NDUFA1 | | |
| NFKBIA | | |
| CCR5 | | |
| GRPEL1 | | |
| HACD4 | | |
| IFIT2 | | |
| RPL3 | | |
| XAF1 | | |
| NDUFAB1 | | |
| HSPA9 | | |
| COX16 | | |
| AKT1 | | |
| GART | | |
| RBX1 | | |
| SKP1 | | |
| TRAV1-2 | | |
| TRBV19 | | |
| CD45R0 | | |
| IFI6 | | |
| GLUD1/2 | | |
| PSMA2 | | |
| CS | | |
| NPRL3 | | |
| RAE1 | | |
| IRF3 | | |
| TRIM25 | | |
| COX7C | | |
| RARG | | |
| SERINC1 | | |
| TBX21 | | |
| NRF1 | | |
| TRAV8-2 | | |
| TRAV3 | | |
| NME2 | | |
| TNFRSF18 | | |
| SGK3 | | |
| GLS2 | | |
| IKZF4 | | |
| CD45RA | | |
| HLA-E | | |
| IL10RA | | |
| UBE21 | | |
| RELA | | |
| EOMES | | |
| NKG7 | | |
| TSC2 | | |
| FH | | |
| TRBV11-3 | | |
| ATP5MF | | |
| MAP2K7 | | |
| IFI30 | | |
| PTGER4 | | |
| MTHFD1 | | |
| ATG7 | | |
| TRBV11-2 | | |
| IDO1 | | |
| IGF1R | | |
| FYN | | |
| SDHB | | |
| PTPN6 | | |
| NR3C1 | | |
| SMARCA4 | | |
| CTNNB1 | | |
| JAK2 | | |
| NAA20 | | |
| CD3D | | |
| PYCR2 | | |
| TGIF2 | | |
| AKT2 | | |
| UQCRQ | | |
| PSMB10 | | |
| NME1 | | |
| PRICKLE3 | | |
| TIMM23 | | |
| UBA5 | | |
| TRAF3 | | |
| IL32 | | |
| CDC42 | | |
| TRIM26 | | |
| DIABLO | | |
| LTA | | |
| PPP2R5D | | |
| ARAF | | |
| GNAI2 | | |
| PDHA1 | | |
| STK11 | | |
| MCAT | | |
| ERAP2 | | |
| ACVR1B | | |
| ACSF2 | | |
| PSMA3 | | |
| MAX | | |
| RDH14 | | |
| MTOR | | |
| PRKAB2 | | |
| TNFRSF10B | | |
| TGFB1 | | |
| TIMM17A | | |
| NCR1 | | |
| PML | | |
| TRBV11-1 | | |
| ADD1 | | |
| TFAM | | |
| SEC22B | | |
| ADAR | | |
| NSD2 | | |
| HAVCR2 | | |
| NMT1 | | |
| PARP1 | | |
| TET2 | | |
| MYC | | |
| PSMA6 | | |
| OAT | | |
| NOTCH1 | | |
| OPAl | | |
| CKAP5 | | |
| TRAV18 | | |
| BCL2 | | |
| TFDP1 | | |
| SHMT2 | | |
| TPR | | |
| ADORA2A | | |
| PPAT | | |
| OAS2 | | |
| SMAD2 | | |
| STAT5A | | |
| STAM | | |
| TRBV10-2 | | |
| NFATC1 | | |
| OXSM | | |
| LPAR6 | | |
| CISH | | |
| FOXO1 | | |
| KLRD1 | | |
| CD8A | | |
| IDH3A | | |
| PIDD1 | | |
| OAS1 | | |
| NEK2 | | |
| LEF1 | | |
| SERINC3 | | |
| SLC2A1 | | |
| KIR3DL1/2 | | |
| TRBV14 | | |
| PIK3C3 | | |
| RAI1 | | |
| TRBV7-4 | | |
| ICOS | | |
| SLAMF6 | | |
| DLL1 | | |
| DOCK2 | | |
| UBE2V1 | | |
| IFNAR1 | | |
| TGFBR2 | | |
| CASP3 | | |
| CREB1 | | |
| SLC25A6 | | |
| WDR45 | | |
| CALM1 | | |
| COX19 | | |
| NDUFB9 | | |
| CD3E | | |
| IL6ST | | |
| TRBV29-1 | | |
| CPT1A | | |
| PTPRC | | |
| TRIM33 | | |
| PPT2 | | |
| PTK2B | | |
| WAS | | |
| MAP3K7 | | |
| TP53 | | |
| AFDN | | |
| SLC25A20 | | |
| MX1 | | |
| ATP5MG | | |
| PRDM1 | | |
| SMAD5 | | |
| HDAC8 | | |
| CD84 | | |
| ACACA | | |
| STAT4 | | |
| LAG3 | | |
| KYAT1 | | |
| CDC26 | | |
| SOCS4 | | |
| SMURF1 | | |
| CTSD | | |
| SOCS5 | | |
| USP15 | | |
| PYCR3 | | |
| IFNGR1 | | |
| CMIP | | |
| VAV3 | | |
| GFER | | |
| SERPINB9 | | |
| MID1IP1 | | |
| CXCR6 | | |
| TRIM34 | | |
| TICAM1 | | |
| PIK3R1 | | |
| PPP3CA | | |
| CD4 | | |
| TRBV7-3 | | |
| FASLG | | |
| CCR2 | | |
| NCAPD2 | | |
| MTHFS | | |
| TNF | | |
| TRAV8-1 | | |
| PCCA | | |
| STAT5B | | |
| CLCF1 | | |
| MINOS1 | | |
| GRK2 | | |
| IL4R | | |
| TRAT1 | | |
| SLC2A11 | | |
| ATG14 | | |
| ACOT2 | | |
| ACVR2A | | |
| HDAC7 | | |
| HLA-DRA | | |
| OASL | | |
| SOCS2 | | |
| RPL23 | | |
| ALDH8A1 | | |
| JAK1 | | |
| PRKCB | | |
| PLCB2 | | |
| CD80 | | |
| GZMH | | |
| KLRC1/2 | | |
| CD3G | | |
| GATA3 | | |
| IL2RG | | |
| ACTN1 | | |
| TCF7 | | |
| TRGV3/5 | | |
| VAV1 | | |
| PDK1 | | |
| LCK | | |
| IL12RB1 | | |
| ALDH3A2 | | |
| SH3BP2 | | |
| NCAPG2 | | |
| HLA-DRB1 | | |
| TRDV3 | | |
| PTGER2 | | |
| SLC27A3 | | |
| LILRB3 | | |
| UBE2F | | |
| IL16 | | |
| BCL2L1 | | |
| PLCG1 | | |
| KLRK1 | | |
| JUNB | | |
| CXCR3 | | |
| ZAP70 | | |
| BLK | | |
| IL18BP | | |
| GZMM | | |
| ITGB2 | | |
| BID | | |
| IL6R | | |
| TYROBP | | |
| AURKA | | |
| IKZF2 | | |
| RORA | | |
| CD160 | | |
| CCR4 | | |
| RNASEL | | |
| MR1 | | |
| SELPLG | | |
| CEACAM1 | | |
| TRAF5 | | |
| CXCR5 | | |
| ENTPD1 | | |
| IL21R | | |
| TRGV8 | | |
| TRGV2 | | |
| TLR2 | | |
| TRBV6-4 | | |
| CXCL8 | | |
| TRGC2 | | |
| IKBKE | | |
| IFITM3 | | |
| SELL | | |
| HK2 | | |
| OAS3 | | |
| PECAM1 | | |
| TGFBR1 | | |
| TOX | | |
| IL23R | | |
| KLRG1 | | |
| FCGR3A/B | | |
| MAML2 | | |
| MKI67 | | |
| CD40LG | | |
| VSIR | | |
| PRF1 | | |
| CD244 | | |
| TRGV4 | | |
| GZMA | | |
| NCR3 | | |
| CD274 | | |
| XCL1/2 | | |
| TRGC1 | | |
| NT5E | | |
| PPARD | | |
| IL7R | | |
| CYBB | | |
| ZBTB16 | | |
| CCR6 | | |
| LTB | | |
| TIMP1 | | |
| IFNG | | |
| LAIR1 | | |
| KLRB1 | | |
| GZMK | | |
| RORC | | |
| CCL4/L1 | | |
| ITGAM | | |
| MAML3 | | |
| CX3CR1 | | |
| CTSW | | |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

What is claimed:

1. An immune cell comprising an exogenous cargo, wherein the immune cell has a molecular profile comprising an expression level of a gene or protein within a log₂ fold change of 3 of the level of the gene or protein in a control immune cell at 24 hours post cargo delivery, and wherein the gene or protein is in the Activator Protein 1 (AP-1) signaling pathway.

2. The immune cell of claim 1, wherein the expression level of the gene or protein is within a log₂ fold change of 2 of the level of the gene or protein in the control immune cell, or wherein the expression level of the gene or protein is within a log₂ fold change of 1 of the level of the gene or protein in the control immune cell.

3. The immune cell of claim 1, wherein the exogenous cargo comprises a nucleic acid, a small molecule, a protein, a polypeptide, or a combination thereof.

4. The immune cell of claim 3, wherein the nucleic acid comprises messenger ribonucleic acid (mRNA), small interfering RNA (siRNA), short hairpin RNA (shRNA), deoxyribonucleic acid (DNA), or any combination thereof.

5. The immune cell of claim 1, wherein the expression of the gene or protein in the AP-1 signaling pathway comprises Fos (v-fos FBJ murine osteosarcoma viral oncogene homolog, FBJ murine osteosarcoma viral oncogene homolog), Jun (v-jun avian sarcoma virus 17 oncogene homolog), or a combination thereof.

6. The immune cell of claim 1, wherein the gene or protein in the AP-1 signaling pathway comprises Fos, Jun, FosB (FBJ murine osteosarcoma viral oncogene homolog B), BATF (Basic leucine zipper transcription factor ATF-like), BATF3 (Basic leucine zipper transcriptional factor ATF-like 3), or combinations thereof.

7. The immune cell of claim 5, wherein Fos comprises human Fos comprising the nucleic acid sequence of SEQ ID NO: 1, and wherein Jun comprises human Jun comprising the nucleic acid sequence of SEQ ID NO: 2.

8. The immune cell of claim 1, wherein the cargo comprises messenger ribonucleic acid (mRNA).

9. The immune cell of claim 8, wherein the mRNA encodes a chimeric antigen receptor (CAR).

10. The immune cell of claim 9, wherein the CAR targets CD19 (cluster of differentiation 19) “CD19 CAR”).

11. The immune cell of claim 10, wherein CD19 CAR comprises the mRNA sequence of SEQ ID NO: 6 or SEQ ID NO: 8.

12. The immune cell of claim 10, wherein the CD19 CAR comprises the protein sequence of SEQ ID NO: 7 or SEQ ID NO: 9.

13. The immune cell of claim 1, wherein the expression of the gene or protein in the AP-1 signaling pathway in the immune cell comprising the exogenous cargo is about a log₂ fold change of −3 compared to the control immune cell.

14. The immune cell of claim 1, wherein the expression of the gene or protein in the AP-1 signaling pathway in the immune cell comprising the exogenous cargo is about a log₂ fold change of −2 compared to the control immune cell.

15. The immune cell of claim 1, wherein the expression of the gene or protein in the AP-1 signaling pathway in the immune cell comprising the exogenous cargo is about a log₂ fold change of −1 compared to the control immune cell.

16. The immune cell of claim 1, wherein the immune cell comprises at least two exogenous cargos.

17. The immune cell of claim 1, wherein the immune cell comprising exogenous cargo do not exhibit T-cell exhaustion or T-cell anergy phenotype.

18. The immune cell of claim 1, wherein the immune cell comprising exogenous cargo comprises an unstimulated immune cell.

19. An immune cell comprising an exogenous cargo, wherein the immune cell secretes at least one cytokine at a level within a log₂ fold change of 3 compared to the level of an immune cell that has not experienced a cell engineering process.

20. An immune cell of claim 19, wherein the immune cell secretes at least one cytokine at a level within a log₂ fold change of 2 compared to the level of the immune cell that has not experienced a cell engineering process.

21. An immune cell of claim 19, wherein the immune cell secretes at least one cytokine at a level within a log₂ fold change of 1 compared to the level of the immune cell that has not experienced a cell engineering process.

22. The immune cell of claim 19, wherein the cytokine comprises human IL-2 (interleukin 2) comprising the nucleic acid sequence of SEQ ID NO: 17, human IL-8 (interleukin 8) comprising the nucleic acid sequence of SEQ ID NO: 18, or a combination thereof.

23. The immune cell of claim 19, wherein the cytokines comprise IFN-γ (interferon gamma), IL-2 (interleukin 2), TNFα(tumor necrosis factor alpha), IL-8 (interleukin 8), GM-CSF (Granulocyte-macrophage colony-stimulating factor), IL-10 (interleukin 10), MIP-1α(macrophage inflammatory protein 1 alpha), MIP-1β (macrophage inflammatory protein 1 beta), IL-17A (interleukin 17A), Fractalkine, or ITAC (Interferon—inducible T Cell Alpha Chemoattractant).

24. A method of delivering an exogenous cargo across a plasma membrane of a non-adherent immune cell, comprising,
providing a population of non-adherent cells; and
contacting the population of cells with a volume of an isotonic aqueous solution, the aqueous solution including the exogenous cargo and an alcohol at greater than 0.2 percent (v/v) concentration, wherein an immune function of the non-adherent immune cell comprises a phenotype of a cell that has not experienced a cell engineering step, wherein the immune function is selected from (i) cytokine release; (ii) gene expression; and (iii) metabolic rate.

25. The method of claim 24, wherein the alcohol is greater than 0.5 percent (v/v) concentration.

26. The method of claim 24, wherein the alcohol is greater than 2 percent (v/v) concentration.

27. The method of claim 24, wherein the alcohol is greater than 5 percent (v/v) concentration.

28. The method of claim 24, wherein the alcohol is greater than 10 percent (v/v) concentration.

29. The method of claim 24, wherein the immune cell is not activated prior to cargo delivery.

30. The method of claim 24, wherein the immune cell has not been contacted with a ligand of CD3, CD28, or a combination thereof, prior to contacting the immune cell with the exogenous cargo.

31. The method of claim 24, further comprising at least two exogenous cargos.

32. The method of claim 31, wherein the at least two exogenous cargos are simultaneously.

33. The method of claim 31, wherein the at least two exogenous cargos are sequentially delivered.

34. A method of delivering at least two exogenous cargos across the plasma membrane of a non-adherent immune cell, comprising,
providing a population of non-adherent cells, and using at least two intracellular delivery methods selected from (i) contacting the population of cells with a volume of an isotonic aqueous solution, the aqueous solution including the exogenous cargo and an alcohol at greater than 0.5 percent (v/v) concentration, (ii) viral transduction, (iii), electroporation or (iv) nucleofection, and thereby delivering the two exogenous cargos to the immune cell.

35. The method of claim 34, wherein the intracellular delivery methods comprise contacting the population of cells with a volume of an isotonic aqueous solution, the aqueous solution including the exogenous cargo and an alcohol at greater than 0.5 percent (v/v) concentration followed by viral transduction.

36. The method of claim 34, wherein the intracellular delivery methods comprise viral transduction followed by contacting the population of cells with a volume of an isotonic aqueous solution, the aqueous solution including the exogenous cargo and an alcohol at greater than 0.5 percent (v/v) concentration.


SEQUENCE LISTING (40 sequences)

SEQ ID NO: 1 - DNA, 2084 bp, Homo sapiens
aaccgcatctgcagcgagcaactgagaagccaagactgagccggcggccgcggcgcagcgaacgagcagtgaccgtgctcctacccagctctgcttcacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggacttctgcacggacctggccgtctccagtgccaacttcattcccacggtcactgccatctcgaccagtccggacctgcagtggctggtgcagcccgccctcgtctcctctgtggccccatcgcagaccagagcccctcaccctttcggagtccccgccccctccgctggggcttactccagggctggcgttgtgaagaccatgacaggaggccgagcgcagagcattggcaggaggggcaaggtggaacagttatctccagaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaa

SEQ ID NO: 2 - DNA, 3338 bp, Homo sapiens
gacatcatgggctatttttaggggttgactggtagcagataagtgttgagctcgggctggataagggctcagagttgcactgagtgtggctgaagcagcgaggcgggagtggaggtgcgcggagtcaggcagacagacagacacagccagccagccaggtcggcagtatagtccgaactgcaaatcttattttcttttcaccttctctctaactgcccagagctagcgcctgtggctcccgggctggtgtttcgggagtgtccagagagcctggtctccagccgcccccgggaggagagccctgctgcccaggcgctgttgacagcggcggaaagcagcggtacccacgcgcccgccgggggaagtcggcgagcggctgcagcagcaaagaactttcccggctgggaggaccggagacaagtggcagagtcccggagcgaacttttgcaagcctttcctgcgtcttaggcttctccacggcggtaaagaccagaaggcggcggagagccacgcaagagaagaaggacgtgcgctcagcttcgctcgcaccggttgttgaacttgggcgagcgcgagccgcggctgccgggcgccccctccccctagcagcggaggaggggacaagtcgtcggagtccgggcggccaagacccgccgccggccggccactgcagggtccgcactgatccgctccgcggggagagccgctgctctgggaagtgagttcgcctgcggactccgaggaaccgctgcgcccgaagagcgctcagtgagtgaccgcgacttttcaaagccgggtagcgcgcgcgagtcgacaagtaagagtgcgggaggcatcttaattaaccctgcgctccctggagcgagctggtgaggagggcgcagcggggacgacagccagcgggtgcgtgcgctcttagagaaactttccctgtcaaaggctccggggggcgcgggtgtcccccgcttgccagagccctgttgcggccccgaaacttgtgcgcgcagcccaaactaacctcacgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgaagagagaccgtcgggggctgaggggcaacgaagaaaaaaaataacacagagagacagacttgagaacttgacaagttgcgacggagagaaaaaagaagtgtccgagaactaaagccaagggtatccaagttggactgggttgcgtcctgacggcgcccccagtgtgcacgagtgggaaggacttggcgcgccctcccttggcgtggagccagggagcggccgcctgcgggctgccccgctttgcggacgggctgtccccgcgcgaacggaacgttggacttttcgttaacattgaccaagaactgcatggacctaacattcgatctcattcagtattaaaggggggagggggagggggttacaaactgcaatagagactgtagattgcttctgtagtactccttaagaacacaaagcggggggagggttggggaggggcggcaggagggaggtttgtgagagcgaggctgagcctacagatgaactctttctggcctgccttcgttaactgtgtatgtacatatatatattttttaatttgatgaaagctgattactgtcaataaacagcttcatgcctttgtaagttatttcttgtttgtttgtttgggtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatactagctatctaggtggagttgaaagagttaagaatgtcgattaaaatcactctcagtgcttcttactattaagcagtaaaaactgttctctattagactttagaaataaatgtacctgatgtacctgatgctatggtcaggttatactcctcctcccccagctatctatatggaattgcttaccaaaggatagtgcgatgtttcaggaggctggaggaaggggggttgcagtggagagggacagcccactgagaagtcaaacatttcaaagtttggattgtatcaagtggcatgtgctgtgaccatttataatgttagtagaaattttacaataggtgcttattctcaaagcaggaattggtggcagattttacaaaagatgtatccttccaatttggaatcttctctttgacaattcctagataaaaagatggcctttgcttatgaatatttataacagcattcttgtcacaataaatgtattcaaataccaaaaaaaaaaaaaaaaa

SEQ ID NO: 3 - protein, 302 aa, Homo sapiens
MFQAFPGDYDSGSRCSSSPSAESQYLSSVDSFGSPPTAAASQECAGLGEMPGSFVPTVTAITTSQDLQWLVQPTLISSMAQSQGQPLASQPPVVDPYDMPGTSYSTPGMSGYSSGGASGSGGPSTSGTTSGPGPARPARARPRRPREETETDQLEEEKAELESEIAELQKEKERLEFVLVAHKPGCKIPYEEGPGPGPLAEVRDLPGSAPAKEDGFSWLLPPPPPPPLPFQTSQDAPPNLTASLFTHSEVQVLGDPFPVVNPSYTSSFVLTCPEVSAFAGAQRTSGSDQPSDPLNSPSLLAL

SEQ ID NO: 4 - protein, 125 aa, Homo sapiens
MPHSSDSSDSSFSRSPPPGKQDSSDDVRRVQRREKNRIAAQKSRQRQTQKADTLHLESEDLEKQNAALRKEIKQLTEELKYFTSVLNSHEPLCSVLAASTPSPPEVVYSAHAFHQPHVSSPRFQP

SEQ ID NO: 5 - protein, 127 aa, Homo sapiens
MSQGLPAAGSVLQRSVAAPGNQPQPQPQQQSPEDDDRKVRRREKNRVAAQRSRKKQTQKADKLHEEYESLEQENTMLRREIGKLTEELKHLTEALKEHEKMCPLLLCPMNFVPVPPRPDPVAGCLPR

SEQ ID NO: 6 - DNA, 1455 bp, Artificial Sequence
  CD19 CAR mRNA
atggctctcccagtgactgccctactgcttcccctagcgctgctgctgcatgcggcgcgcccggacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcggaggcggatctggcggcggaggatctgggggaggcggctctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 7 - protein, 484 aa, Artificial Sequence
  CD19 CAR protein
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - DNA, 1552 bp, Artificial Sequence
  CD19 CAR mRNA
tgatatccagatgacccagaccaccagcagcctgtctgcctctctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacttctgggtgctcgtggttgttggcggagtgctggcctgttacagcctgctggttaccgtggccttcatcatcttttgggtccgaagcaagcggagccggctgctgcactccgactacatgaacatgacccctagacggcccggacctaccagaaagcactaccagccttacgctcctcctagagacttcgccgcctacagatccaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagggatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaagataagtcgacaatcaa

SEQ ID NO: 9 - protein, 512 aa, Artificial Sequence
  CD19 CAR protein
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 846 bp, Artificial Sequence
  E195R/D269H TRAIL (DR5)-variant
atggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggagcgaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatgcaccatgaagccagttttttcggggcctttttagttggctaa

SEQ ID NO: 11 - protein, 281 aa, Artificial Sequence [sequence omitted]

SEQ ID NO: 12 - DNA, 21 bp, Artificial Sequence
  PDCD1 reverse primer
agggactgagggtggaaggtc

SEQ ID NO: 13 - DNA, 35 bp, Artificial Sequence
  c-Myc primer
ggatccgaattcatgcccctcaacgttagcttcac

SEQ ID NO: 14 - DNA, 34 bp, Artificial Sequence
  Klf4 primer
ggatccgaattcatggctgtcagcgacgcgctgc

SEQ ID NO: 15 - DNA, 34 bp, Artificial Sequence
  Oct4 primer
ggatccgaattcatggcgggacacctggcttcgg

SEQ ID NO: 16 - DNA, 34 bp, Artificial Sequence
  Sox2 primer
ggatccgaattcatgtacaacatgatggagacgg

SEQ ID NO: 17 - DNA, 1047 bp, Homo sapiens
cgaattcccctatcacctaagtgtgggctaatgtaacaaagagggatttcacctacatccattcagtcagtctttgggggtttaaagaaattccaaagagtcatcagaagaggaaaaatgaaggtaatgttttttcagacaggtaaagtctttgaaaatatgtgtaatatgtaaaacattttgacacccccataatatttttccagaattaacagtataaattgcatctcttgttcaagagttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaaaaaaaaaaa

SEQ ID NO: 18 - DNA, 2058 bp, Homo sapiens
acaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagaggtaagttatatattttttaatttaaatttttcatttatcctgagacatataatccaaagtcagcctataaatttctttctgttgctaaaaatcgtcattaggtatctgcctttttggttaaaaaaaaaaggaatagcatcaatagtgagtttgttgtactcatgaccagaaagaccatacatagtttgcccaggaaattctgggtttaagcttgtgtcctatactcttagtaaagttctttgtcactcccagtagtgtcctattttagatgataatttctttgatctccctatttatagttgagaatatagagcatttctaacacatgaatgtcaaagactatattgacttttcaagaaccctactttccttcttattaaacatagctcatctttatatttttaattttattttagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaa

SEQ ID NO: 19 - protein, 1368 aa, Streptococcus pyogenes
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQTYNQLFEENPINASRVDAKAILSARLSKSRRLENLIAQLPGEKKNGLFGNLIALLLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDKEMIEERLKKYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSDILKEYPVENTQLQNEKLYLYYLQNGRDMYVDQELDINRLSDYDVDHIVPQSFLKDDSIDNKVLTRSDKNRGKSDNVPSEEVVKKMKNYWKQLLNAKLITQRKFDNLTKAERGGLSELDKAGFIKRQLVETRQITKHVAQILDSRMNTKYDENDKLIREVRVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 20 - protein, 994 aa, Staphylococcus agnetis
MNNYILGLDIGITSVGYGIVDSDTREIKDAGVRLFPEANVDNNEGRRSKRGARRLKRRRIHRLDRVKHLLAEYNLLDLTNIPKSTNPYQIRVKGLNEKLSKDELVIALLHIAKRRGIHNVNVMMDDNDSGNELSTKDQLKKNAKALSDKYVCELQLERFEQDYKVRGEKNRFKTEDFVREARKLLETQSKFFEIDQTFIMRYIDLVETRREYFEGPGKGSPFGWEGNIKKWFEQMMGHCTYFPEELRSVKYAYSAELFNALNDLNNLVITRDEEAKLNYGEKFQIIENVFKQKKTPNLKQIAKEIGVSETDIKGYRVNKSGKPEFTQFKLYHDLKNIFEDSKYLNDVQLMDNIAEIITIYQDPESIIKELNQLPELLSEKEKEKISALSGYAGTHRLSLKCINLLLDDLWESSLNQMELFTKLNLKPKKIDLSQQHKIPIKLVDDFILSPVVKRAFIQSIQVVNAIIDKYGLPEDIIIELARENNSDDRRKFLNQLQKQNAETRKQVEKVLREYGNDNAKRIVQKIKLHNMQEGKCLYSLKDIPLEDLLKNPNHYEVDHIIPRSVAFDNSMHNKVLVRAEENSKKGNRTPYQYLNSSESSLSYNEFKQHILNLSKTKDRITKKKREYLLEERDINKYDVQKEFINRNLVDTRYATRELTSLLKAYFSANNLDVKVKTINGSFTNYLRKVWKFDKDRNKGYKHHAEDALIIANADFLFKHNKKLRNINKVLDAPSKEVDKKRVTVQSEDEYNQMFEDTQKAQAIKKFEIRKFSHRVDKKPNRQLIKDTLYSTRNIDGIEYVVESIKDIYSVNNDKVKTKFKKDPHRLLMYRNDPQTFEKFEKVFKQYESEKNPFAKYYEETGEKIRKFSKTGQGPYINKIKYLRERLGRHCDVTNKYINSRNKIVQLKIYSYRFDIYQYGNNYKMITISYIDLEQKSNYYYISREKYEQKKKDKQIDDSYKFIGSFYKNDIINYNGEMYRVIGVNDSEKIKFSLI

SEQ ID NO: 21 - protein, 1110 aa, Artificial Sequence
  Staphylococcus aureus Cas9 sequence
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTREQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEERQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSYPYDVPDYASGFANELGPRLMGK

SEQ ID NO: 22 - protein, 1235 aa, Artificial Sequence
  Candidatus Methanomethylophilus alvus Mx1201 Cas12a
MHTGGLLSMDAKEFTGQYPLSKTLRFELRPIGRTWDNLEASGYLAEDRHRAECYPRAKELLDDNHRAFLNRVLPQIDMDWHPIAEAFCKVHKNPGNKELAQDYNLQLSKRRKEISAYLQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWKN

SEQ ID NO: 23 - protein, 1227 aa, Artificial Sequence
  Candidatus Methanomethylophilus alvus isolate MGYG-HGUT-02456 Cas12a
MDAKEFTGQYPLSKTLRFELRPIGRTWDNLEASGYLAEDRHRAECYPRAKELLDDNHRAFLNRVLPQIDMDWHPIAEAFCKVHKNPGNKELAQDYNLQLSKRRKEISAYLQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWKN

SEQ ID NO: 24 - protein, 1238 aa, Artificial Sequence
  Candidatus Methanoplasma termitum strain MpT1 chromosome Cas12a
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDEVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIKRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLPRVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 25 - DNA, 20 bp, Artificial Sequence
  TRAC gRNA
agagtctctcagctggtaca

SEQ ID NO: 26 - DNA, 20 bp, Artificial Sequence
  PDCD1 targeting gRNA
gtctgggcggtgctacaact

SEQ ID NO: 27 - protein, 331 aa, Homo sapiens
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 28 - DNA, 1929 bp, Homo sapiens
aggagccgccgccagtggagggccgggcgctgcggccgcggccggggcgggcgcagggccgagcggacgggggggcgcgggccccccgggaggccgcggccactcccccccgggccggcgcggcgggggaggcggaggatggaaacacccttctacggcgatgaggcgctgagcggcctgggcggcggcgccagtggcagcggcggcagcttcgcgtccccgggccgcttgttccccggggcgcccccgacggccgcggccggcagcatgatgaagaaggacgcgctgacgctgagcctgagtgagcaggtggcggcagcgctcaagcctgcggccgcgccgcctcctacccccctgcgcgccgacggcgcccccagcgcggcaccccccgacggcctgctcgcctctcccgacctggggctgctgaagctggcctcccccgagctcgagcgcctcatcatccagtccaacgggctggtcaccaccacgccgacgagctcacagttcctctaccccaaggtggcggccagcgaggagcaggagttcgccgagggcttcgtcaaggccctggaggatttacacaagcagaaccagctcggcgcgggcgcggccgctgccgccgccgccgccgccgccggggggccctcgggcacggccacgggctccgcgccccccggcgagctggccccggcggcggccgcgcccgaagcgcctgtctacgcgaacctgagcagctacgcgggcggcgccgggggcgcggggggcgccgcgacggtcgccttcgctgccgaacctgtgcccttcccgccgccgccacccccaggcgcgttggggccgccgcgcctggctgcgctcaaggacgagccacagacggtgcccgacgtgccgagcttcggcgagagcccgccgttgtcgcccatcgacatggacacgcaggagcgcatcaaggcggagcgcaagcggctgcgcaaccgcatcgccgcctccaagtgccgcaagcgcaagctggagcgcatctcgcgcctggaagagaaagtgaagaccctcaagagtcagaacacggagctggcgtccacggcgagcctgctgcgcgagcaggtggcgcagctcaagcagaaagtcctcagccacgtcaacagcggctgccagctgctgccccagcaccaggtgcccgcgtactgagtccgcgcgcggggcgcatgcgcggccaccctccccaaggggcgggctcgcgggggggtgtcgtgggcgccccggacttggagagggtgcggccctggggaccccccctccccgagtgtgcccaggaactcagagagggcgcggcccccggggattccccccccccgagggtgcccaggactcgacaagctggaccccctgctcccgggggggcgagcgcatgacccccccgccctcgcgctgcctctttcccccgcgcggccgccccgtgttgcacaaacccgcgcgtctcggctgcccctttgtacaccgcgccgcggaagggggctccgagggggcgcagcctcaaaccctgcctttcctttacttttacttttttttttttttctttggaagagagaagaacagagtgttcgattctgccctatttatgtttctactcgggaacaaacgttggttgtgtgtgtgtgtgttttcttgtgttggttttttaaagaaatgggaagaagaaaaaaaaattctccgcccctttcctcgatctcgctccccccttcggttctttcgaccggtcccccctcccttttttgttctgttttgttttgttttgctacgagtccacattcctgtttgtaatccttggttcgcccggttttctgttttcagtaaagtctcgttacgccagctcggctctccgcctccttcttcccccgccggggcctggcgggctgggcggggcctggttcgctt

SEQ ID NO: 29 - DNA, 3775 bp, Homo sapiens
cattcataagactcagagctacggccacggcagggacacgcggaaccaagacttggaaacttgattgttgtggttcttcttgggggttatgaaatttcattaatcttttttttttccggggagaaagtttttggaaagattcttccagatatttcttcattttcttttggaggaccgacttactttttttggtcttctttattactcccctccccccgtgggacccgccggacgcgtggaggagaccgtagctgaagctgattctgtacagcgggacagcgctttctgcccctgggggagcaacccctccctcgcccctgggtcctacggagcctgcactttcaagaggtacagcggcatcctgtgggggcctgggcaccgcaggaagactgcacagaaactttgccattgttggaacgggacgttgctccttccccgagcttccccggacagcgtactttgaggactcgctcagctcaccggggactcccacggctcaccccggacttgcaccttacttccccaacccggccatagccttggcttcccggcgacctcagcgtggtcacaggggcccccctgtgcccagggaaatgtttcaggctttccccggagactacgactccggctcccggtgcagctcctcaccctctgccgagtctcaatatctgtcttcggtggactccttcggcagtccacccaccgccgcggcctcccaggagtgcgccggtctcggggaaatgcccggttccttcgtgcccacggtcaccgcgatcacaaccagccaggacctccagtggcttgtgcaacccaccctcatctcttccatggcccagtcccaggggcagccactggcctcccagcccccggtcgtcgacccctacgacatgccgggaaccagctactccacaccaggcatgagtggctacagcagtggcggagcgagtggcagtggtgggccttccaccagcggaactaccagtgggcctgggcctgcccgcccagcccgagcccggcctaggagaccccgagaggagacgctcaccccagaggaagaggagaagcgaagggtgcgccgggaacgaaataaactagcagcagctaaatgcaggaaccggcggagggagctgaccgaccgactccaggcggagacagatcagttggaggaagaaaaagcagagctggagtcggagatcgccgagctccaaaaggagaaggaacgtctggagtttgtgctggtggcccacaaaccgggctgcaagatcccctacgaagaggggcccgggccgggcccgctggcggaggtgagagatttgccgggctcagcaccggctaaggaagatggcttcagctggctgctgccgcccccgccaccaccgcccctgcccttccagaccagccaagacgcaccccccaacctgacggcttctctctttacacacagtgaagttcaagtcctcggcgaccccttccccgttgttaacccttcgtacacttcttcgtttgtcctcacctgcccggaggtctccgcgttcgccggcgcccaacgcaccagcggcagtgaccagccttccgatcccctgaactcgccctccctcctcgctcggtgaactctttagacacacaaaacaaacaaacacatgggggagagagacttggaagaggaggaggaggaggagaaggaggagagagaggggaagagacaaagtgggtgtgtggcctccctggctcctccgtctgaccctctgcggccactgcgccactgccatcggacaggaggattccttgtgttttgtcctgcctcttgtttctgtgccccggcgaggccggagagctggtgactttggggacagggggtgggaaggggatggacacccccagctgactgttggctctctgacgtcaacccaagctctggggatgggtggggaggggggcgggtgacgcccaccttcgggcagtcctgtgtgaggatgaagggacgggggtgggaggtaggctgtggggtgggctggagtcctctccagagaggctcaacaaggaaaaatgccactccctacccaatgtctcccacacccaccctttttttggggtgcccaggttggtttcccctgcactcccgaccttagcttattgatcccacatttccatggtgtgagatcctctttactctgggcagaagtgagcccccccttaaagggaattcgatgcccccctagaataatctcatccccccacccgacttcttttgaaatgtgaacgtccttccttgactgtctagccactccctcccagaaaaactggctctgattggaatttctggcctcctaaggctccccaccccgaaatcagcccccagccttgtttctgatgacagtgttatcccaagaccctgccccctgccagccgaccctcctggccttcctcgttgggccgctctgatttcaggcagcaggggctgctgtgatgccgtcctgctggagtgatttatactgtgaaatgagttggccagattgtggggtgcagctgggtggggcagcacacctctggggggataatgtccccactcccgaaagcctttcctcggtctcccttccgtccatcccccttcttcctcccctcaacagtgagttagactcaagggggtgacagaaccgagaagggggtgacagtcctccatccacgtggcctctctctctctcctcaggaccctcagccctggcctttttctttaaggtcccccgaccaatccccagcctaggacgccaacttctcccaccccttggcccctcacatcctctccaggaaggcagtgaggggctgtgacatttttccggagaagatttcagagctgaggctttggtacccccaaacccccaatatttttggactggcagactcaaggggctggaatctcatgattccatgcccgagtccgcccatccctgaccatggttttggctctcccaccccgccgttccctgcgcttcatctcatgaggatttctttatgaggcaaatttatattttttaatatcggggggtggaccacgccgccctccatccgtgctgcatgaaaaacattccacgtgccccttgtcgcgcgtctcccatcctgatcccagacccattccttagctatttatccctttcctggtttccgaaaggcaattatatctattatgtataagtaaatatattatatatggatgtgtgtgtgtgcgtgcgcgtgagtgtgtgagcgcttctgcagcctcggcctaggtcacgttggccctcaaagcgagccgttgaattggaaactgcttctagaaactctggctcagcctgtctcgggctgacccttttctgatcgtctcggcccctctgattgttcccgatggtctctctccctctgtcttttctcctccgcctgtgtccatctgaccgttttcacttgtctcctttctgactgtccctgccaatgctccagctgtcgtctgactctgggttcgttggggacatgagattttattttttgtgagtgagactgagggatcgtagatttttacaatctgtatctttgacaattctgggtgcgagtgtgagagtgtgagcagggcttgctcctgccaaccacaattcaatgaatccccgacccccctaccccatgctgtacttgtggttctctttttgtattttgcatctgaccccggggggctgggacagattggcaatgggccgtcccctctccccttggttctgcactgttgccaataaaaagctcttaaaaacgc

SEQ ID NO: 30 - DNA, 953 bp, Homo sapiens
caagagagagagagagcgtgcaagccccaaagcgagcgacatgtccctttggggagcagtccctctgcaccccagagtgaggaggacgcaggggtcagaggtggctacagggcaggcagaggaggcacctgtagggggtggtgggctggtggcccaggagaagtcaggaagggagcccagctggtgacaagagagcccagaggtgcctggggctgagtgtgagagcccggaagatttcagccatgcctcacagctccgacagcagtgactccagcttcagccgctctcctccccctggcaaacaggactcatctgatgatgtgagaagagttcagaggagggagaaaaatcgtattgccgcccagaagagccgacagaggcagacacagaaggccgacaccctgcacctggagagcgaagacctggagaaacagaacgcggctctacgcaaggagatcaagcagctcacagaggaactgaagtacttcacgtcggtgctgaacagccacgagcccctgtgctcggtgctggccgccagcacgccctcgccccccgaggtggtgtacagcgcccacgcattccaccaacctcatgtcagctccccgcgcttccagccctgagcttccgatgcggggagagcagagcctcgggaggggcacacagactgtggcagagctgcgcccatcccgcagaggcccctgtccacctggagacccggagacagaggcctggacaaggagtgaacacgggaactgtcacgactggaagggcgtgaggcctcccagcagtgccgcagcgtttcgaggggcgtgtgctggaccccaccactgtgggttgcaggcccaatgcagaagagtattaagaaagatgctcaagtcccatggcacagagcaaggcgggcagggaacggttatttttctaaataaatgctttaaaagaaaaaaaaaaaaaaa

SEQ ID NO: 31 - DNA, 992 bp, Homo sapiens
ggggcagacgtgggacgggaaggacggctgccgggactggcgcgcggggacactgggccgacgcgtggagtagcggggagagcgggaagcctgagggggcggggccggcgcgaggccgtgggtgcggcacgaggatgccggcggcgggacagcgcccgtaggcagccccacgggcagggcgcgcgggcggggcggggcgggccgggccagaggagcgcccggcatgtcgcaagggctcccggccgccggcagcgtcctgcagaggagcgtcgcggcgcccgggaaccagccgcagccgcagccgcagcagcagagccctgaggatgatgacaggaaggtccgaaggagagaaaaaaaccgagttgctgctcagagaagtcggaagaagcagacccagaaggctgacaagctccatgaggaatatgagagcctggagcaagaaaacaccatgctgcggagagagatcgggaagctgacagaggagctgaagcacctgacagaggcactgaaggagcacgagaagatgtgcccgctgctgctctgccctatgaactttgtgccagtgcctccccggccggaccctgtggccggctgcttgccccgatgaagccggggacactcctctgcccagcaaggagccttggtcattttcatacctgggaggaaggcttttccttcacaattgtatacagggggcacctgtggccaggcctcctcctgggagctccaggaccagccagctgtgttccctgcagactgggctcagcccgacatccaacaggcgccaaactcacagagcccttgtgcagatccagcatggaggccaccctcaggagtgacttctcatccaccctggcagctagtaggttctgctgttatgcagagccatttcctctagaatttggataataaagatgcttattgtctctcccttctccagttctgggaatttacaggcacaatacacttccttttcctggaaaaaaaaaaaa

SEQ ID NO: 32 - protein, 380 aa, Homo sapiens
MMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLAL

SEQ ID NO: 33 - protein, 331 aa, Homo sapiens; identical to SEQ ID NO: 27

SEQ ID NO: 34 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 35 - protein, 95 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKR

SEQ ID NO: 36 - protein, 281 aa, Artificial Sequence
  E195R/D269H TRAIL (DR5 Variant)
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQERIKENTKNDKQMVQYIYKYTSYPDPILLMKSARNSCWSKDAEYGLYSIYQGGIFELKENDRIFVSVTNEHLIDMHHEASFFGAFLVG

SEQ ID NO: 37 - DNA, 63 bp, Artificial Sequence
  Klf4 primer
aaaaaactcgaggcggcgtctgcgtctgcggcgtctgcgaaagtgcctcttcatgtgtaaggc

SEQ ID NO: 38 - DNA, 60 bp, Artificial Sequence
  Oct4 primer
aaaaaagtcgacgcggcgtctgcgtctgcggcgtctgcggtttgaatgcatgggagagcc

SEQ ID NO: 39 - DNA, 59 bp, Artificial Sequence
  Sox2 primer
aaaaaactcgaggcggcgtctgcgtctgcggcgtctgcgcatgtgcgacaggggcagtg

SEQ ID NO: 40 - DNA, 22 bp, Artificial Sequence
  PDCD1 forward primer
agcactgcctctgtcactctcg